(19) United States
(12) Patent Application Publication
(10) Pub. No.: US 2023/0375561 A1
(43) Pub. Date: Nov. 23, 2023

(54) METHODS FOR IDENTIFICATION, STRATIFICATION, AND TREATMENT OF CNS DISEASES

(71) Applicants: Rima F. KADDURAH-DAOUK, Belmont, MA (US); Ranga Rama KRISHNAN, Durham, NC (US); Matthias ARNOLD, Germering (DE); Rebecca BAILLIE, Woodland, CA (US); Xianlin HAN, San Antonio, TX (US); Duke University, Durham, NC (US)

(72) Inventors: Rima F. Kaddurah-Daouk, Belmont, MA (US); Ranga Rama Krishnan, Durham, NC (US); Matthias Arnold, Germering (DE); Rebecca Baillie, Woodland, CA (US); Xianlin Han, San Antonio, TX (US)

(21) Appl. No.: 18/246,977

(22) PCT Filed: Sep. 30, 2021
(86) PCT No.: PCT/US2021/052863

§ 371 Date: Mar. 28, 2023

Related U.S. Application Data
(60) Provisional application No. 63/239,656, filed on Sep. 1, 2021.
(60) Provisional application No. 63/165,395, filed on Mar. 24, 2021.
(60) Provisional application No. 63/085,706, filed on Sep. 30, 2020.

(51) Int. Cl. G01N 33/68; C12Q 1/6883; A61P 25/28; A61P 25/24; A61K 45/06; G01N 33/53; G01N 33/92

(52) U.S. Cl. CPC G01N 33/6812; C12Q 1/6883; A61P 25/28; A61P 25/24; A61K 45/06; G01N 33/5308; G01N 33/92; G01N 2800/304; G01N 2800/52; G01N 2800/50; C12Q 2600/156; G01N 2570/00; C12Q 2600/106

(57) ABSTRACT

Described herein are methods for identifying mitochondrial defects using metabolomics and genetic analyses and using this information to stratify patients and to correct for metabolic defects in a precision medicine approach. One embodiment is a method for isolating and analyzing samples containing one or more mitochondrial biomarker metabolites or genetic markers useful for the analysis, identification, stratification or classification, and treatment of metabolic changes associated with a CNS or neuropsychiatric disease in a subject and therapies useful for the treatment thereof. In one aspect, the biomarker metabolites comprise mitochondrial metabolites including acylcarnitines and endocannabinoids and the genetic analyses focus on metabolic enzymes or transport mechanisms.

### CROSS-REFERENCE TO RELATED APPLICATIONS

This application claims priority to U.S. Provisional Patent Application Nos. 63/085,706, filed on Sep. 30, 2020; 63/165,395, filed on Mar. 24, 2021; and 63/239,656, filed on Sep. 1, 2021, each of which is incorporated by reference herein in its entirety.

### FEDERALLY SPONSORED RESEARCH

This invention was made with government support under grant numbers MH 108348, U01 AG061359, RF1AG057452, and RF1AG051550 awarded by the National Institutes of Mental Health (NIH/NIMH). The government has certain rights in the invention.

### TECHNICAL FIELD

Described herein are methods for identifying mitochondrial defects using metabolomics and genetic analyses and using this information to stratify patients and to correct for metabolic defects in a precision medicine approach. One embodiment is a method for isolating and analyzing samples containing one or more mitochondrial biomarker metabolites or genetic markers useful for the analysis, identification, stratification or classification, and treatment of metabolic changes associated with a CNS or neuropsychiatric disease in a subject and therapies useful for the treatment thereof. In one aspect, the biomarker metabolites comprise mitochondrial metabolites including acylcarnitines and endocannabinoids and the genetic analyses focus on metabolic enzymes or transport mechanisms.

### BACKGROUND

Mitochondria are organelles within the cell that generate the energy that sustains ATP production, which is necessary for cell activity and survival. The oxidation of metabolites occurs in the mitochondria, mainly through the Krebs/TCA cycle and through the β-oxidation of fatty acids. Mitochondria are also the main generators of reactive oxygen species (ROS) and calcium homeostasis. They regulate cellular pathways, including the release of neurotransmitters from neurons and glial cells. Mitochondria also respond to perturbations in cell homeostasis during conditions of compromise and stress. Mitochondrial dysfunction has been implicated in human neurodegenerative and neuropsychiatric diseases.

Neurodegenerative diseases, while heterogeneous, are all characterized by the progressive loss of specific neuronal populations and circuits in the central nervous system (CNS). These are seemingly triggered by deficiencies in the mitochondria. Defects in mitochondrial function have been implicated in the pathogenesis of several CNS diseases, including Alzheimer's disease (AD), Parkinson's disease (PD), Huntington's disease (HD) and amyotrophic lateral sclerosis (ALS), among others. The impact of defects may be both central and peripheral. Neurodegeneration-related mitochondrial changes include the oxidative stress increase of intracellular ROS formation, disruption of mitochondrial membranes and cristae with decreased ATP production, accumulation of altered proteins leading to changes in cell structure and function, disruption of calcium hemostasis, permeabilization of mitochondrial membrane, and processes triggered by neuroinflammation with a “switch” in mitochondrial function which is an important contributor to the transition from a normal physiological condition to a degenerative condition. Different CNS diseases have different etiologies, including genetic, environmental and lifestyle factors, as well as chemical exposure and other influences. However, all neurodegenerative and neuropsychiatric diseases seem to share the theme of mitochondrial dysfunction. These changes all impact critical pathways involved in cell growth and differentiation, cellular signaling, cell cycle control, and cell apoptosis. These features, together with the appearance of mitochondrial dysfunctions early in disease onset and their contribution to disease progression, seem to be distinctive features of neurodegeneration and neuropsychiatric diseases such as depression and bipolar disorder.

What is needed are approaches to define which mitochondrial dysfunction is present in which patients afflicted with a neurodegenerative or psychiatric disease or susceptible for developing neuropsychiatric disease and to be able to tailor metabolic interventions based on specific mitochondrial dysfunctions that is applying a precision medicine approach for the treatment of disease disorders based on mitochondrial dysfunction and methods for targeting mitochondrial pathways related to oxidative stress, mitochondrial biogenesis, and mitochondrial membrane permeability and dynamics for the treatment of various neurodegenerative diseases.

### SUMMARY

One embodiment described herein is a method for the classification and treatment of a CNS disease in a subject, the method comprising one or more of the following: identifying and stratifying subjects afflicted with a CNS disease into subgroups based on their metabolic profiles, biomarker metabolites and ratios of biomarker metabolites that define unique metabolic conditions related to change in mitochondrial function and common identity among subgroups of subjects; evaluating the trajectory of disease within each stratified subgroup of subjects and their response to a therapeutic treatment; identifying defects in transport and/or biosynthesis breakdown of biomarker metabolites within a metabolic pathway or across metabolic pathways using ratios of biomarker metabolites to inform about changes in enzyme activities or transporters; and identifying genetic bases of metabolic profile characteristics or defects (SNPs/genetic variants in key enzymes and transporters) using mGWAS analysis. In one aspect, the method further comprises comprising one or more of the following: using combined metabotype and genotype data to better stratify subjects with neuropsychiatric diseases and to inform about mechanisms and treatment selection; suggesting a therapeutic approach to correct metabolic defects in metabolic profile in stratified subgroups of subjects; and comparing and contrasting metabolic defects noted in inborn errors of metabolism that have neurological and CNS deficits and using knowledge gained in treatment of inborn errors of metabolism to inform treatment for CNS diseases. In another aspect, the method further comprises comprising administering to the stratified subgroups of subjects an effective amount of a therapy to prevent and/or treat the CNS disease affected by specified genetic metabolic defects.

Another embodiment described herein is a method for stratifying and treating a subject having a neurological disorder, or at risk of developing a neurological disorder, based on the subject's mitochondrial metabolic profile, the method comprising: obtaining a sample from the subject; measuring the concentration levels and calculating the ratios of one or more mitochondrial biomarker metabolites in the sample, wherein the one or more mitochondrial biomarker metabolites are selected from carnitine, short-chain acylcarnitines, medium-chain acylcarnitines, or long-chain acylcarnitines; ketone bodies; amino acids, branched chain amino acids; biogenic amines; glycerophospholipids; sphingolipids; short-chain fatty acids; endocannabinoids; eicosanoids; other metabolites of glycolysis, TCA cycle, fatty acid beta-oxidation, urea cycle, or ketogenesis; or combinations thereof; determining if the subject has a mitochondrial metabolic defect related to disrupted acylcarnitine homeostasis, TCA cycle, glycolysis, fatty acid beta-oxidation, ketogenesis, urea cycle, or combinations thereof based on the measured concentration levels and calculated ratios of the one or more mitochondrial biomarker metabolites in the sample as compared to a control sample; and stratifying the subject into a subgroup of subjects, wherein an individual subgroup of subjects is defined by a unique and specific mitochondrial metabolic profile based on the measured concentration levels and calculated ratios of the one or more mitochondrial biomarker metabolites in the sample as compared to a control sample and the mitochondrial metabolic defect determined for the subject. In one aspect, the method further comprises administering to the subgroup of subjects an effective amount of a therapy to treat the neurological disease, wherein the therapy is determined by the unique and specific mitochondrial metabolic profile of the subgroup of subjects. In another aspect, the biomarker metabolite comprises one or more of: Carnitine; Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine); or combinations thereof. In another aspect, the mitochondrial metabolic defect is related to disrupted acylcarnitine homeostasis and comprises: lower concentration levels of all acylcarnitines and higher ratios of carnitine/C3:0, carnitine/C5:0, carnitine/C10:0, carnitine/C16:0, and carnitine/C18:1 acylcarnitines; lower ratios of short and medium chain vs. long chain acylcarnitines (including C3:0/C16:0, C5:0/C16:0, C10:0/C16:0, C3:0/C18:1, C5:0/C18:1, and C10:0/C18:1); lower ratios of short chain vs. medium and long chain acylcarnitines (including C3:0/C10:0, C5:0/C10:0, C3:0/C16:0, C5:0/C16:0, C3:0/C18:1 and C5:0/C18:1); or lower ratios of odd-numbered short chain acylcarnitines vs. even-numbered short chain acylcarnitines (e.g., C6) and medium and long chain acylcarnitines (including C3:0/C6:0, C5:0/C6:0, C3:0/C10:0, C5:0/C10:0, C3:0/C16:0, C5:0/C16:0, C3:0/C18:1 and C5:0/C18:1). In another aspect, the mitochondrial metabolic defect is related to disrupted acylcarnitine homeostasis and comprises: Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); and Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine). In another aspect, the therapy comprises one or more compounds selected from peroxisome proliferator-activated receptor (PPAR) agonists, PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acid: Even (C₆-C₁₂), medium chain fatty acid: Odd chain fatty acids (C₇, C₉), branched chain amino acids, carnitine, acetyl carnitine, propionylcarnitine, short chain acylcarnitines (C₂₋₅), cofactors NAD Flavin FAD, and combinations thereof. In another aspect, the mitochondrial metabolic defect comprises a deficiency in amino acids (e.g., branched chain amino acids) and/or short chain fatty acids. In another aspect, the therapy comprises one or more compounds selected from branched chain amino acids, propionic acid, ketone bodies, short chain acylcarnitines (C₂₋₅), medium chain acylcarnitines, analogs thereof, and combinations thereof. In another aspect, the neurological disorder is a CNS disorder, depression, or treatment resistant depression.

Another embodiment described herein is a method for stratifying and treating a subject having a neurological or CNS disorder, or at risk of developing a CNS or neurological disorder, based on the subject's mitochondrial metabolic profile, the method comprising: obtaining a sample from the subject; measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof in the sample; and determining if the subject has a mitochondrial metabolic defect related to disrupted acylcarnitine homeostasis, TCA cycle, glycolysis, fatty acid beta-oxidation, ketogenesis, urea cycle, or combinations thereof based on the measured expression level and/or activity of mitochondrial enzymes and/or transporters in the sample as compared to a control sample. In one aspect, the method further comprises stratifying the subject into a subgroup of subjects, wherein an individual subgroup of subjects is defined by a unique and specific mitochondrial metabolic profile based on the measured expression level and activity of mitochondrial enzymes and/or transporters in the sample as compared to a control sample and the mitochondrial metabolic defect determined for the subject. In another aspect, the method further comprises administering to the subgroup of subjects an effective amount of a therapy to treat the neurological disease, wherein the therapy is determined by the unique and specific mitochondrial metabolic profile of the subgroup of subjects. In another aspect, the mitochondrial enzyme and/or transporter comprises gam ma-butyrobetaine hydroxylase 1 (BBOX1), organic cation transporter novel family member 2 (OCTN2), very long chain acylCoA dehydrogenase (VLCAD), medium chain acylCoA dehydrogenase (MCAD), short chain acylCoA dehydrogenase (SCAD), carnitine palmitoyltransferase1/2 (CPT1/2), carnitine-acylcarnitine translocase (CACT), carnitine octanoyltransferase, acetyl-CoA carboxylase1/2 (ACC1/2), ATP citrate synthase (ACLY), peroxisome proliferator-activated receptor (PPARα/PPARγ), or combinations thereof. In another aspect, the mitochondrial metabolic defect is related to disrupted acylcarnitine homeostasis and comprises: CPT defects: lower concentration levels of all acylcarnitines and higher ratios of carnitine/C3:0, carnitine/C5:0, carnitine/C10:0, carnitine/C16:0, and carnitine/C18:1 acylcarnitines; VLCAD defects: lower ratios of short and medium chain vs. long chain acylcarnitines (including C3:0/C16:0, C5:0/C16:0, C10:0/C16:0, C3:0/C18:1, C5:0/C18:1, and C10:0/C18:1); MCAD defects: lower ratios of short chain vs. medium and long chain acylcarnitines (including C3:0/C10:0, C5:0/C10:0, C3:0/C16:0, C5:0/C16:0, C3:0/C18:1 and C5:0/C18:1); or SCAD defects: lower ratios of odd-numbered short chain acylcarnitines vs. even-numbered short chain acylcarnitines (e.g., C6) and medium and long chain acylcarnitines (including C3:0/C6:0, C5:0/C6:0, C3:0/C10:0, C5:0/C10:0, C3:0/C16:0, C5:0/C16:0, C3:0/C18:1 and C5:0/C18:1). In another aspect, the therapy comprises one or more compounds selected from peroxisome proliferator-activated receptor (PPAR) agonists, PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acid: Even (C₆-C₁₂), medium chain fatty acid: Odd chain fatty acids (C₇, C₉), branched chain amino acids, carnitine, acetyl carnitine, propionylcarnitine, short chain acylcarnitines (C₂₋₅), cofactors NAD Flavin FAD, and combinations thereof. In another aspect, the mitochondrial metabolic defect is related to disrupted acylcarnitine homeostasis and comprises disrupted BBOX1, OCTN2, and/or CPT1/2 expression and/or activity.

Another embodiment described herein is a method for treating a CNS or neuropsychiatric disease in a subject, the method comprising: obtaining a sample from the subject; determining the presence, concentration levels, and ratios of one or more biomarker metabolites related to mitochondrial function in the sample from the subject; comparing the presence, concentration levels, and ratios of one or more biomarker metabolites related to mitochondrial function in the sample from the subject to the presence, concentration levels, and ratios of the one or more biomarker metabolites in a control sample; and determining if the subject has a CNS or neuropsychiatric disorder, or has an increased risk of developing a CNS or neuropsychiatric disorder when the concentration levels and ratios of the one or more biomarker metabolites in the sample from the subject are different from (greater than or less than) the concentration levels and ratios of the one or more biomarker metabolites in a control sample. In one aspect, the method further comprises: stratifying the subject into a subgroup of subjects based on the concentration levels and ratios of the one or more biomarker metabolites related to mitochondrial function in the sample, wherein each subgroup of subjects is defined by a unique and specific mitochondrial metabolic profile; and administering to the subgroup of subjects an effective amount of a therapy to treat the CNS or neuropsychiatric disease, wherein the therapy is determined by the unique and specific mitochondrial metabolic profile of each subgroup of subjects.

Another embodiment described herein is a method for targeting mitochondrial pathways related to oxidative stress, mitochondrial biogenesis, and mitochondrial membrane permeability and dynamics in a subject suffering from, or at risk of suffering from, one or more neurodegenerative diseases, the method comprising: obtaining a sample from the subject and determining the concentration levels and ratios of one or more mitochondrial biomarker metabolites in the sample from the subject; determining if the subject has a neurodegenerative disease, or has an increased risk of developing a neurodegenerative disease when the concentration levels and ratios of the one or more mitochondrial biomarker metabolites in the sample from the subject are different from (greater than or less than) the concentration levels and ratios of the one or more mitochondrial biomarker metabolites in a control sample; stratifying the subject into a subgroup of subjects based on the concentration levels and ratios of the one or more mitochondrial biomarker metabolites in the sample, wherein each subgroup of subjects is defined by a unique and specific mitochondrial metabolic profile; and administering to the subgroup of subjects an effective amount of a therapy to treat the neurodegenerative disease, wherein the therapy is determined by the unique and specific mitochondrial metabolic profile of the subgroup of subjects.

Another embodiment described herein is a method for preparing and analyzing a sample containing a biomarker metabolite useful for the analysis and identification of metabolic changes associated with a CNS or neuropsychiatric disease in a subject, the method comprising: obtaining a sample from a subject; performing metabolic analysis on the sample to detect the presence and concentration of one or more biomarker metabolites; comparing the presence and concentration levels of one or more biomarker metabolites in the sample from the subject to the concentration levels of the one or more biomarker metabolites in a control sample; determining whether the presence and concentration levels of one or more biomarker metabolites in the sample from the subject correlate with the incidence of a CNS or neuropsychiatric disease, or an increased risk of a CNS or neuropsychiatric disease.

Another embodiment described herein is a method for stratifying and treating a subject having a neurological disorder, or at risk of developing a neurological disorder, based on the subject's mitochondrial metabolic profile, the method comprising: obtaining a sample from the subject; measuring the concentration levels and calculating the ratios of one or more mitochondrial biomarker metabolites in the sample, wherein the one or more mitochondrial biomarker metabolites are selected from carnitine, short-chain acylcarnitines, medium-chain acylcarnitines, or long-chain acylcarnitines; ketone bodies; amino acids, branched chain amino acids; biogenic amines; glycerophospholipids; sphingolipids; short-chain fatty acids; endocannabinoids; eicosanoids; other metabolites of glycolysis, TCA cycle, fatty acid beta-oxidation, urea cycle, or ketogenesis; or combinations thereof; measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof in the sample; and determining if the subject has a mitochondrial metabolic related to disrupted acylcarnitine homeostasis, TCA cycle, glycolysis, fatty acid beta-oxidation, ketogenesis, urea cycle, or combinations thereof based on the measured concentration levels and calculated ratios of the one or more mitochondrial biomarker metabolites in the sample as compared to a control sample and/or genetic defect in one or more mitochondrial enzymes and/or transporters involved in acylcarnitine biosynthesis and transport, TCA cycle, glycolysis, fatty acid beta-oxidation, ketogenesis, urea cycle, or combinations thereof; and stratifying the subject into a subgroup of subjects, wherein an individual subgroup of subjects is defined by a unique and specific mitochondrial metabolic profile based on the measured concentration levels and calculated ratios of the one or more mitochondrial biomarker metabolites in the sample as compared to a control sample and the mitochondrial metabolic defect determined for the subject; administering to the subgroup of subjects an effective amount of a therapy to treat the neurological disease, wherein the therapy is determined by the unique and specific mitochondrial metabolic profile of the subgroup of subjects. In one aspect, the therapy comprises one or more compounds selected from branched chain amino acids, propionic acid, ketone bodies, short chain acylcarnitines (C₂₋₅), medium chain acylcarnitines, analogs thereof, and combinations thereof. In another aspect, the neurological disorder is a CNS disorder, depression, or treatment resistant depression.

Another embodiment described herein is a therapy useful in any of the methods described herein. In one embodiment, the therapy comprises one or more repurposed compounds that improve mitochondrial energetics to treat CNS or neuropsychiatric diseases. In another embodiment, therapy comprises one or more repurposed compounds that modulate mitochondrial energetics to treat neurodegenerative diseases. In another embodiment, the therapy comprises one or more of HU-210; CP 55940; Win 55212-2; anandamide; 2-AG; Noladin ether; virodhamine; oleoylethanolamide; palmitoylethanolamide; PPARγ Agonists; PPARβ/δ Agonists; dual and pan PPAR agonists; chiglitazar (CS038); AVE0847; aleglitazar (R1439); 5-substituted 2-benzoylamino-benzoic acid derivatives (BVT-142); O-arylmandelic acid derivatives; azaindole-α-alkyloxyphenylpropionic acid; amide substituted/α-substituted β-phenylpropionic acid derivatives; 2-alkoxydihydrocinnamate derivative; α-aryloxy-α-methylhydrocinnamic acids (LYS1029); TZD18; α-aryloxyphenyl acetic acid derivatives; PLX249; muraglitazar; mesaglitazar; naveglitazar; ragaglitazar; farglitazar; imiglitazar; netoglitazone; compound 3q JTT-501; MK0767; KRP-297; AZD6610; (atorvastatin+ezetimibe+fenofibrate); (fenofibrate+pravastatin sodium); (fenofibrate+rosuvastatin calcium); (fenofibrate+rosuvastatin); (fenofibrate+simvastatin); (fenofibrate+pitavastatin); (gliclazide+metformin hydrochloride+pioglitazone hydrochloride); (gliclazide+metformin hydrochloride+rosiglitazone); (gliclazide SR+metformin hydrochloride SR+pioglitazone hydrochloride); (gliclazide SR+metformin SR+pioglitazone); glimepiride+metformin SR+pioglitazone; (metformin ER+pioglitazone); (metformin hydrochloride+pioglitazone); (metformin hydrochloride+rosiglitazone maleate); (metformin hydrochloride+pioglitazone hydrochloride); (fenofibrate+metformin hydrochloride); (gliclazide+rosiglitazone); (glimepiride+pioglitazone); (alogliptin benzoate+pioglitazone hydrochloride); ciprofibrate; fenofibrate; gemfibrozil; bezafibrate SR; clinofibrate; clofibrate; clofibrate; choline fenofibrate; saroglitazar; lobeglitazone; zaltoprofen; pemafibrate; pemafibrate+tofogliflozin; MA-0211; REN-001; EHP-101; ZYH-7; elafibranor; NC-2400; MA-0217; T-3D959; CHS-131; efatutazone; OMS-405; seladelpar lysine; leriglitazone hydrochloride; CS-038; AU-9; BIO-201; BIO-203; BR-101549; CDIM-9; CNB-001; ELB-00824; ETI-059; KR-62980; MA-0204; PLX-300; RB-394; SR-10171; sulindac; ZG-0588; A-91; AIC-47; CDE-001; CDIM-1; CDIM-5; CDIM-7; OP-601; (azilsartan+pioglitazone hydrochloride); ADC-3277; ADC-8316; ARH-049020; arhalofenate; ATX-08001; AVE-0897; AZD-6610; BP-1107; CG-301269; CLC-3000; CLC-3001; CNX-013B2; CP-778875; CS-1050; CXR-1002; DB-900; DJ-5; DRF-10945; etalocib; farglitazar; GED-0507; indeglitazar; K-111; KD-3010; KD-3020; KRP-101; LY-518674; LY-518674; mesalamine; NIP-222; NP-774; NS-220; PAM-1616; PBI-4547; PBI-4547; PBI-4547; peroxibrate; PN-2034; PPM-201; PPM-202; romazarit; metformin; triheptanoin; ketone bodies; short chain fatty acids; methanoic acid; ethanoic acid; propanoic acid; butanoic acid; 2-methyl propanoic acid; pentanoic acid; 3-methyl butanoic acid; medium chain fatty acids; medium even (C6-C12) chain fatty acids; medium odd (C7, C9) chain fatty acids; fatty acid ethanolamides; cannabinoids; branched chain amino acids; nicotinamide adenine dinucleotide (NAD+/NADH, NADP+/NADPH); riboflavin; flavin adenine dinucleotide (FAD); EC5026; GSK2256294; AR9281; TPPU; t-TUCB; Dronabinol; Epidiolex; Δ9-tetrahydrocannabinol; cannabidiol; Δ9-tetrahydrocannabinol+cannabidiol; SSR411298; PF-04457845; JNJ-42165279; URB597 (KDS-4103); ST4070 (Alfasigma); Nabilone; Um-PEA (FSD201); NEO6860; OEA (RiduZone); or combinations thereof. In another embodiment, therapy comprises one or more of: Δ⁹-tetrahydrocannabinol (Δ⁹-THC; Dronabinol); Δ⁸-tetrahydrocannabinol (Δ⁸-THC); exo-tetrahydrocannabinol (Exo-THC); Δ⁹-tetrahydrocannabinol naphtoylester (Δ⁹-THC-NE); Δ⁸-tetrahydrocannabinol naphtoylester (Δ⁸-THC-NE); exo-tetrahydrocannabinol naphtoylester (Exo-THC-NE); Δ⁹-tetrahydrocannabinolic acid (THCA-A, THCA-B); Δ⁸-tetrahydrocannabinolic acid (Δ⁸-THCA-A, Δ⁸-THCA-B); (−)-cannabidiol ((−)-CBD)/(+)-cannabidiol ((+)-CBD); cannabidiol-2′,6′-dimethyl ether (CBDD); 4-monobromo cannabidiol (4-MBO-CBD); cannabidiolic acid (CBDA); cannabiquinone (CBQ); nabilone; cannabivarin (CBNV); cannabivarinic acid (CBNVA); cannabivarin naphtoylester (CBNV-NE); tetrahydrocannabivarin (Δ⁹-THCBV); Δ⁸-tetrahydrocannabivarin (Δ⁸-THCBV); Δ⁹-tetrahydrocannabivarin naphtoylester (Δ⁹-THCV-NE); Δ⁸-tetrahydrocannabivarin naphtoylester (Δ⁸-THCV-NE); Δ⁹-tetrahydrocannabivarinic acid (Δ⁹-THCVA); Δ⁸-tetrahydrocannabivarinic acid (Δ⁸-THCVA); (−)-cannabidivarin ((−)-CBDV)/(+)-cannabidivarin ((+)-CBDV)); cannabidivarinic acid (CBDVA); cannabidivarin quinone (CBQV); cannabidibutol (CBDB); cannabidibutolic acid (CBDBA); cannabidibutol naphtoylester (CBDB-NE); Δ⁹-tetrahydrocannabidutol (Δ⁹-THCBDB); Δ⁸-tetrahydrocannabidutol (Δ⁸-THCBDB); Δ⁹-tetrahydrocannabidutolic acid (Δ⁹-THCBDBA); Δ⁸-tetrahydrocannabidutolic acid (Δ⁸-THCBDBA); Δ⁹-tetrahydrocannabidutol naphtoylester (Δ⁹-THCB-NE); Δ⁸-tetrahydrocannabidutol naphtoylester (Δ⁸-THCB-NE); cannabibutol (CBB); cannabibutolic acid (CBBA); Δ⁹-tetrahydrocannabibutol (Δ⁹-THCB); Δ⁸-tetrahydrocannabibutol (Δ⁸-THCB); Δ⁹-tetrahydrocannabibutoic acid (Δ⁹-THCBA); Δ⁸-tetrahydrocannabibutolic acid (Δ⁸-THCBA); Δ⁹-tetrahydrocannabibutol naphtoylester (Δ⁹-THCB-NE); Δ⁸-tetrahydrocannabibutol naphtoylester (Δ⁸-THCB-NE); cannabinol (CBN); cannabinolic acid (CBNA); 3-butylcannabinol (CBNB); 3-butylcannabinolic acid (CBNBA); cannabielsoin (CBE); cannabicitran (CBT); cannabicyclol (CBL); cannabicyclolic acid (CBLA); cannabicyclol butyl (CBLB); cannabicyclol butyric acid (CBLBA); cannabicyclolvarin (CBLV); cannabicyclolvarinic acid (CBLVA); cannabigerol (CBG); cannabigerolic acid (CBGA); cannabigerol butyl (CBGB); cannabigerol butyric acid (CBGBA); cannabichromene (CBC); cannabichromenic acid (CBCA); cannabichromene butyl (CBCB); cannabichromene butyric acid (CBCBA); cannabigerivarin (CBGV); cannabigerivarinic acid (CBGVA); cannabichromevarin (CBCV); cannabichromevarinic acid (CBCVA); other cannabinoids, or pharmaceutically acceptable salts, acids, esters, amides, hydrates, solvates, prodrugs, isomers, stereoisomers, tautomers, derivatives thereof, or combinations thereof. In another embodiment, the therapy further comprises an anti-depressant selected from selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof. In another embodiment, the therapy further comprises one or more of citalopram, escitalopram, duloxetine, fluoxetine, paroxetine, sertraline, trazodone, lorazepam, oxazepam, aripiprazole, clozapine, haloperidol, olanzapine, quetiapine, risperidone, ziprasidone, amitriptyline, amoxapine, desipramine, doxepin, imipramine, nortriptyline, protriptyline, trimipramine, or combinations thereof. In another embodiment, the therapy further comprises ketamine or esketamine. In another embodiment, the therapy comprises one or more compounds selected from cannabinoids, cannabinoid-like compounds, carnitine, L-acetyl carnitines, and combinations thereof.

Another embodiment described herein is a method for stratifying and treating metabolic changes related to mitochondrial dysfunction, the method comprising: (a) obtaining a sample from one or more subjects suffering from a CNS disease or disorder; (b) analyzing the concentrations of mitochondrial metabolite biomarkers; (c) identifying mitochondrial metabolite biomarkers with abnormal concentration levels or abnormal concentration ratios compared to those of normal subjects; (d) identifying, analyzing, and cataloging enzymes or genes that are implicated in the metabolic, anabolic, catabolic, or transport pathways of the mitochondrial metabolite biomarkers with abnormal concentrations; and (e) administering diet changes, drugs, or combinations thereof to modulate the mitochondrial metabolite biomarkers with abnormal concentrations. In one aspect, the identifying, analyzing, and cataloging enzymes and genes that are implicated in the metabolic, anabolic, catabolic, or transport pathways of the mitochondrial metabolite biomarkers with abnormal concentrations comprises (a) performing a genetic screen analysis of the samples using GWAS analysis, Mendelian randomization analyses, univariable Mendelian randomization analyses, and/or multivariable Mendelian randomization analyses; (b) measuring the concentration levels and calculating the ratios of one or more mitochondrial biomarker metabolites in the samples, wherein the one or more mitochondrial biomarker metabolites; (c) comparing the genetic screen analyses to the measured concentration levels and calculated ratios of the one or more mitochondrial biomarker metabolites in the sample; (d) identifying a genetic basis of mitochondrial metabolic profile characteristics or any metabolic profile defects (SNPs/genetic variants in key enzymes and transporters) based on the genetic screen analyses; (e) determining if there is a causative genetic association between the measured concentration levels and calculated ratios of the one or more mitochondrial biomarker metabolites and depression in the subjects; and (f) stratifying the subjects afflicted with depression into subgroups based on their metabolic profiles, biomarker metabolites and ratios of biomarker metabolites, genetic screen analyses, and identified genetic associations.

Another embodiment described herein is a method for identifying genetics changes related to mitochondrial dysfunction, the method comprising: (a) obtaining a sample from one or more subjects suffering from a CNS disease or disorder; (b) performing a genetic screen analysis of the samples using GWAS analysis, Mendelian randomization analyses, univariable Mendelian randomization analyses, and/or multivariable Mendelian randomization analyses; (c) measuring the concentration levels and calculating the ratios of one or more mitochondrial biomarker metabolites in the samples, wherein the one or more mitochondrial biomarker metabolites; (d) comparing the genetic screen analyses to the measured concentration levels and calculated ratios of the one or more mitochondrial biomarker metabolites in the sample; (e) identifying a genetic basis of mitochondrial metabolic profile characteristics or any metabolic profile defects (SNPs/genetic variants in key enzymes and transporters) based on the genetic screen analyses; (f) determining if there is a causative genetic association between the measured concentration levels and calculated ratios of the one or more mitochondrial biomarker metabolites and depression in the subjects; and (g) stratifying the subjects afflicted with depression into subgroups based on their metabolic profiles, biomarker metabolites and ratios of biomarker metabolites, genetic screen analyses, and identified genetic associations.

Another embodiment described herein is a genomics-based method for identifying genetic causative mechanisms in subjects suffering from depression, the method comprising: (a) obtaining a sample from the subjects; (b) performing a genetic screen analysis of the samples using GWAS analysis, Mendelian randomization analyses, univariable Mendelian randomization analyses, and/or multivariable Mendelian randomization analyses; (c) measuring the concentration levels and calculating the ratios of one or more mitochondrial biomarker metabolites in the samples, wherein the one or more mitochondrial biomarker metabolites are selected from carnitine, short-chain acylcarnitines, medium-chain acylcarnitines, or long-chain acylcarnitines; or combinations thereof; (d) comparing the genetic screen analyses to the measured concentration levels and calculated ratios of the one or more mitochondrial biomarker metabolites in the sample; (e) identifying a genetic basis of mitochondrial metabolic profile characteristics or any metabolic profile defects (SNPs/genetic variants in key enzymes and transporters) based on the genetic screen analyses; (f) determining if there is a causative genetic association between the measured concentration levels and calculated ratios of the one or more mitochondrial biomarker metabolites and depression in the subjects; and (g) stratifying the subjects afflicted with depression into subgroups based on their metabolic profiles, biomarker metabolites and ratios of biomarker metabolites, genetic screen analyses, and identified genetic associations. In one aspect, low concentration levels of the short-chain acylcarnitines (C2, C3) and high levels of medium-chain acylcarnitines (C8, C10) are identified to have a causative association in depression. In another aspect, high levels of medium-chain acylcarnitines (C8, C10) indicate inborn errors of metabolism. In another aspect, the affected genes/enzymes that generate a metabolic defect may include electron transfer flavoprotein dehydrogenase (ETFDH) and/or medium-chain acyl-CoA dehydrogenase (ACADM). In another aspect, the affected genes/genes that generate a metabolic defect include short-chain acyl-CoA dehydrogenase (ACADS) and/or long-chain acyl-CoA dehydrogenase (ACADL). In one aspect, the method further comprises administering to the stratified subjects an effective amount of a therapy to treat the depression, wherein the therapy is determined by the genetic basis of mitochondrial metabolic profile characteristics or metabolic profile defects and the association with measured levels of mitochondrial biomarker metabolites. In another aspect, the therapy comprises compounds that inhibit the enzymes ETFDH or ACADM.

Another embodiment described herein is a method for discriminating or distinguishing between mild and severe depression in a subject using any of the methods as described herein. In one aspect, the method comprises performing a genetic screen analysis of the subject using GWAS analysis, Mendelian randomization analyses, univariable Mendelian randomization analyses, and/or multivariable Mendelian randomization analyses. In another aspect, the method comprises measuring the concentration levels and calculating the ratios of one or more mitochondrial biomarker metabolites. In another aspect, the method comprises measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof. In another aspect, the method comprises identifying causative associations between the measured concentration levels and calculated ratios of the one or more mitochondrial biomarker metabolites and depression.

Another embodiment described herein is a method for genetically screening for defects in metabolic processes (e.g., acylcarnitine biosynthesis and transport; TCA cycle, glycolysis, fatty acid beta-oxidation, ketogenesis, urea cycle, or electron transport chain) associated with CNS diseases and disorders using any of the methods as described herein. In one aspect, the method comprises identifying SNPs and gene variants of key enzymes and transporters involved in mitochondrial metabolic processes. In another aspect, the defective metabolic process comprises acylcarnitine biosynthesis wherein low concentration levels of the short-chain acylcarnitines (C2, C3) and high levels of medium-chain acylcarnitines (C8, C10) are identified to have a causative association in depression. In another aspect, high levels of medium-chain acylcarnitines (C8, C10) indicate inborn errors of metabolism that might mimic in part common mechanisms with neuropsychiatric diseases. Other medium chain acylcarnitines C12 and enzymes implicated in their regulation are also implicated.

Another embodiment described herein is a method for screening for compounds that modulate the activity of mitochondrial enzymes or transporters (e.g., ETFDH; ACADS; ACADM; ACADL, and other enzyme involved in acylcarnitine regulation) using any of the methods as described herein. In one aspect, the method comprises querying a compound screen to a specific enzyme or transporter; identifying compound hits that interact with the enzyme or transporter; performing quantitative structure/activity relationship analyses; identifying important compound moieties; and optimizing lead compound hits.

Another embodiment described herein is a method for correcting metabolic defects to treat CNS diseases or disorders by administering medications, modulating diet, or providing lifestyle interventions using any of the methods as described herein.

Another embodiment described herein is a method for screening for acylcarnitine homeostasis defects and treating subjects with CNS diseases or disorders by measuring short-, medium-, and long-chain acylcarnitine metabolite levels; genetically screening for SNPs and other genetic variants of key metabolic enzymes and transporters; stratifying subjects based on their metabolic profile and genetic screening; and administering an effective amount of an appropriate therapy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B schematically show the mitochondrial energetics contribution of glycolysis, fatty acid oxidation, and amines to energy production through the citric acid cycle (TCA) and electron respiratory chain (ETC), as well as connections to de novo fatty acid synthesis, cholesterol synthesis, and ketone body production.

FIG. 2 schematically shows the disruption of acylcarnitine homeostasis due to the defects of carnitine biosynthesis and transport (Scenario 1).

FIG. 3 schematically shows the disruption of the acylcarnitine homeostasis due to the defects of fatty acid transport to mitochondria and fatty acid beta oxidation in mitochondria as represented with SCAD deficiency (Scenario 2). The enzymes MCAD, VLCAD, and ETFDH are all key factors in this process as well. MCAD and VLCAD genetic variants may be connected to inborn metabolic errors and other neurological diseases.

FIG. 4 schematically shows the disruption of acylcarnitine homeostasis due to the deficiency in branched amino acids and/or short chain fatty acids which are largely generated from the gut, leading to deficiency in odd-numbered short chain acylcarnitines (Scenario 3).

FIG. 5 schematically shows the potential disruption of acylcarnitine homeostasis due to the defects in biosynthesis of malonylCoA (which is an inhibitor of CPT machinery) through regulation of ACLY and ACC1/2 genes.

FIG. 6 schematically shows the downstream pathways of Triheptanoin in the production of energy (ATP), TCA cycle intermediates (succinyl-CoA), ketone bodies, among others.

FIG. 7 shows a heatmap illustrating the metabolic signature from exposure to the drugs, Escitalopram/Citalopram.

FIG. 8 shows graphs illustrating metabolite concentration levels stratified by remission vs. treatment failure response groups in depressed subjects treated with escitalopram and citalopram across visits from baseline to 8 weeks. Levels (log 2 transformed) of metabolites are shown at pre-treatment and 8 weeks post-treatment. Asterisks indicate statistical significance of mean differences between the two groups (unadjusted p<0.05). Error bars represent standard error of the means. Black stars represent statistical significance at visit. P-values were obtained from linear mixed effect models corrected for age, sex, antidepressant, and 17-item Hamilton Rating Scale for Depression scores.

FIG. 9 shows a heatmap illustrating acylcarnitine metabolomic profiles from clinically defined major depressive phenotypes.

FIG. 10 shows SNP-heritability and pairwise genetic correlation of acylcarnitines. Diagonal: SNP-heritability (h2SNP) estimates. Heatmap: genetic correlation coefficients.

FIG. 11 shows Univariable Mendelian randomization analyses. Exposure: acylcarnitines. Outcome: depression. Odds ratios [ORs] and 95% confidence intervals per SD increase in genetically predicted levels of (log)ACs.

FIG. 12 shows Reversed Univariable Mendelian randomization analyses. Exposure: depression. Outcome: acylcarnitines. Estimates and 95% confidence intervals of change in SD of (log)ACs levels per doubling (2-fold increase) in the prevalence of the exposure.

FIG. 13 shows Multivariable Mendelian Randomization analyses. Joint exposure: acylcarnitines with statistically significant causal estimates in univariable analyses. Outcome: depression. Odds ratios [ORs] and 95% confidence intervals per SD increase in genetically predicted levels of (log)ACs.

FIG. 14 shows a distribution of subject's probability of being in a fasted state in the control and AD groups. Colors show 60% probability cutoffs for the following: red—fasted; blue—non-fasted.

FIG. 15 shows a schematic of plasma differences in oxylipins and endocannabinoids, between control and AD group. Fold changes projected on fatty acids, oxylipin and endocannabinoids metabolic pathway. Only differences with the t-test p<0.05 and FDR correction at q=0.2 are shown. The network presents fatty acids metabolic pathway, including saturates and monounsaturates (SFA and MUFA) and omega 3 and omega 6 fatty acids with oxylipins and endocannabinoids synthesis pathway. Both detected (black font) and not-detected (gray font) metabolites are shown to visualize the coverage of the metabolic pathway in the targeted assay and facilitate data interpretation. Oxylipin metabolizing enzymes are colored by their class: red—lipoxygenase (LOX) and autoxidation pathway; blue—cytochrome p450 (CYP) epoxygenase; green—cyclooxygenase (COX); yellow—N-acylphosphatidylethanolamide-phospholipase D. Node size represents the fold difference, and the color represents the directionality of the difference: orange—higher in AD; light blue—lower in AD. Key enzymes involved in metabolic step are abbreviated next to the edge. Fads—fatty acid desaturase; Elov—fatty acids elongase; sEH—soluble epoxide hydrolase; DH—dehydrogenase. Saturated and monounsaturated fatty acids were not measured in this assay and are indicated only to visualize precursors for measured oxylipins and endocannabinoids.

FIG. 16 shows a schematic of CSF differences in oxylipins and endocannabinoids, between control and AD group. Fold changes projected on fatty acids, oxylipin and endocannabinoids metabolic pathway. Only differences with the t-test p<0.05 and FDR correction at q=0.2 are shown. The network presents fatty acids metabolic pathway, including saturates and monounsaturates (SFA and MUFA) and omega 3 and omega 6 fatty acids with oxylipins and endocannabinoids synthesis pathway. Only metabolites detected in CSF are shown. Oxylipin metabolizing enzymes are colored by their class: red—lipoxygenase (LOX) and autoxidation pathway; blue—cytochrome p450 (CYP) epoxygenase; green—cyclooxygenase (COX); yellow—N-acylphosphatidylethanolamide-phospholipase D. Node size represents the fold difference, and the color represents the directionality of the difference: orange—higher in AD; light blue—lower in AD. Key enzymes involved in metabolic step are abbreviated next to the edge. Fads—fatty acid desaturase; Elov—fatty acids elongase; sEH—soluble epoxide hydrolase; DH—dehydrogenase. Saturated and monounsaturated fatty acids were not measured in this assay and are indicated only to visualize precursors for measured oxylipins and endocannabinoids.

FIG. 17 shows a schematic of the relation between plasma and CSF predictors of AD. Partial least square discriminant analysis (PLS-DA) of AD vs control, utilizing metabolites from both plasma and CSF in predicted fasted samples. Treatment group discrimination is shown by the SCORES (inset) with a plane of discrimination indicated by dashed read line, while metabolites weighting in group discrimination are shown by the LOADINGS. Loading node color indicates metabolite origin (pink for plasma and blue for CSF). Loading node size indicates metabolite variable importance in projection (i.e., VIP). Analysis was performed with all measured metabolites, including specific ratios, but only those with VIP 1.4 are displayed for clarity purpose.

FIG. 18 shows a predictive model for AD with plasma and CSF metabolites. Predictive model built independently for plasma (left), CSF (middle), and plasma+CSF (right). Effect summary shows metabolite model components, sorted by their contribution to the model, with key pathways colored in yellow (fatty acids ethanolamides) and blue (cytochrome p450/soluble epoxide hydrolase pathway). The receiver operating characteristic (ROC) curve for the training set, together with the area under the curve (AUC) and the n for the training (T) and the validation (V) cohorts, are shown in the bottom panel.

FIG. 19 is a schematic showing the biosynthesis pathway of bile acids. Node colors represent primary (blue) and secondary (dark red) bile acids. Node shape represent conjugated (diamond) and unconjugated (oval) bile acids. Node size represents median concentration in the experimental cohort. Cholesterol (top of the pathway) is converted to primary bile acids along two pathways, neutral and acidic. Further, primary bile acids are secreted to the gut, where a portion are modified by the gut bacteria to secondary bile acids. Primary and secondary bile acids are reabsorbed into the blood stream and reenter the liver, where they are conjugated with the amino acids, glycine, or taurine. Conjugated bile acids are then being secreted back to the gut along with primary bile acids. Gut bacteria can cleave conjugated amino acids off bile acids, and freed metabolites are recirculated. Therefore, plasma levels of conjugated bile acids can reflect both liver and gut bacteria activity.

FIG. 20 shows multilinear regression of log(t-Tau/AB42) and the components of AD predictive models, presented in FIG. 13. Analysis performed separately for plasma (upper panel) and CSF (lower panel) AD predictors. Association of individual components are shown in the leverage plots, whereas effect summary contains descriptive statistics for each individual metabolite in the model.

FIG. 21 is a graph showing control and AD group MoCA and log(t-Tau/Aβ42) in accordance with one embodiment of the present disclosure.

FIG. 22 shows a schematic for targeting cytochrome P450 (CYP)/soluble epoxide hydrolase (sEH) and ethanolamide pathways to improve mitochondrial function.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, rats, mice, rabbits, pigs, cows, sheep, goats, horses, dogs, cats, fish, birds, and the like. Typically, the subject is a mammal. A subject also refers to primates (e.g., humans, male or female; infant, adolescent, or adult) or non-human primates. In one embodiment, the subject is a primate. In one embodiment, the subject is a human. The methods and compositions disclosed herein can be used on a sample either in vitro (for example, on isolated cells or tissues) or in vivo in a subject (i.e., living organism, such as a subject). In some embodiments, the subject comprises a human who is suffering from a CNS disease or disorder.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, “treatment,” “therapy” and/or “therapy regimen” refer to the clinical intervention made in response to a disease, disorder (e.g., a CNS disease or disorder), or physiological condition manifested by a subject or to which a subject may be susceptible. The aim of treatment includes the alleviation or prevention of symptoms, slowing or stopping the progression or worsening of a disease, disorder, or condition and/or the remission of the disease, disorder, or condition.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the terms “control,” “reference level,” and “reference” are used interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive index Model (AIM) methodology. Cutoff values (or predetermined cutoff values) may be determined by a receiver operating curve (ROC) analysis from biological samples of the subject group. ROC analysis, as generally known in the biological arts, is a determination of the ability of a test to discriminate one condition from another, e.g., to determine the performance of each marker in identifying a subject having CRC. Alternatively, cutoff values may be determined by a quartile analysis of biological samples of a subject group. For example, a cutoff value may be determined by selecting a value that corresponds to any value in the 25^{th}-75^{th} percentile range, preferably a value that corresponds to the 25^{th} percentile, the 50^{th} percentile or the 75^{th} percentile, and more preferably the 75^{th} percentile. Such statistical analyses may be performed using any method known in the art and can be implemented through any number of commercially available software packages. The healthy or normal levels or ranges for a target or for a protein activity may be defined in accordance with standard practice.

As used herein, “healthy control” or “normal control” refers to a human subject that is not suffering from, or at risk of suffering from, a neuropsychiatric disease or disorder including, but not limited to, depression, anxiety, major depressive disorder, or combinations thereof.

As used herein, the terms “biological sample,” “sample,” or “test sample” refer to any sample in which the presence and/or level of a target is to be detected or determined or any sample comprising an agent or cell as described herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art. In one embodiment, the biological sample comprises blood. In another embodiment, the biological sample comprises plasma. A biological sample may be obtained directly from a subject (e.g., by blood or tissue sampling) or from a third party (e.g., received from an intermediary, such as a healthcare provider or lab technician).

As used herein, the term “biomarker” refers to a naturally occurring biological molecule present in a subject at varying concentrations useful in predicting the risk or incidence of a disease or a condition, such as a neurological disorder. The biomarker can include, but is not limited to, nucleic acids (e.g., DNA), ribonucleic acids (e.g., RNA, including miRNA), proteins, or a polypeptide that is used as an indicator or marker for the disease and/or condition in the subject. Biomarkers may reflect a variety of disease characteristics, including the level of exposure to an environmental or genetic trigger, an element of the disease process itself, and intermediate stage between exposure and disease onset, or an independent factor associated with the disease state, but not causative of pathogenesis. Biomarkers may be used to determine the status of a subject or the effectiveness of a treatment. Biomarker combinations with the most diagnostic utility have both high sensitivity and specificity. In practice, biomarkers and/or specific combinations of biomarkers having both high sensitivity and specificity are not obvious. Evaluation, assessment, and combination of specific biomarkers for diagnosis provide an improved approach to disease treatment.

As used herein, the term “abnormal” refers to biomarker concentration levels, ratios of biomarker concentration levels, or genetic mutations differing from typical i.e., “normal” subjects in a population.

As used herein the terms “stratify” or “stratifying” refer to the identification and ranking of subjects based on one or more metabolic biomarkers or genetic analyses into groups or classifications based on the data obtained. In one aspect, the arrangement is based on the degree of aberrancy or difference in the biomarker concentration, ratios of concentrations, or genetic mutation as compared to normal subjects.

As used herein, the term “administering” an agent, such as a therapeutic entity to an animal or cell, is intended to refer to dispensing, delivering, or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery and administration by the intranasal or respiratory tract route.

As used herein, the term “CNS disease” refers to a host of undesirable conditions affecting neurons in the brain of a subject. Included within the definition of a CNS disease are neuropsychiatric and/or neurodegenerative diseases, depression, treatment resistant depression, anxiety, autism, schizophrenia, schizoaffective disorders, ADHD and the like. Representative examples of such conditions include, without limitation, Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dystonia syndrome, Leigh syndrome, Leber hereditary optic neuropathy (LHON), Parkinsonism, dystonia, motor neuron disease, neuropathy-ataxia and retinitis pigmentosa (NARP), maternal inherited Leigh syndrome (MILS), Friedreich ataxia, hereditary spastic paraplegia, Mohr-Tranebjaerg syndrome, Wilson disease, sporadic Alzheimer's disease, sporadic amyotrophic lateral sclerosis, sporadic Parkinson's disease, autonomic function disorders, hypertension, sleep disorders, neuropsychiatric disorders, depression, autism, schizophrenia, schizoaffective disorder, Korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders. As used herein, the term “movement disorder” includes neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathisia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus.

As used herein, “depression” refers to a mood disorder that causes a persistent feeling of sadness and loss of interest. As used herein, “depression” includes subclinical characteristics associated with depression such as sadness, loss of interest in activities, loss of appetite, anhedonia, insomnia, changes in sleep, difficulty falling asleep, waking during the night, restless sleep, waking too early, sleeping too much, low energy level, lack of concentration, diminished or altered daily behavior, low self-esteem, suicidal thoughts, anxiety coupled with depression, or combinations thereof. As used herein “major depressive disorder” refers to a mental health disorder characterized by persistent feeling of sadness or loss of interest that characterizes major depression can lead to a range of behavioral and physical symptoms causing significant impairment in daily life. These may include depressed mood, loss of interest in activities, changes in sleep, appetite, energy level, concentration, daily behavior, self-esteem, or suicide ideation. In one aspect, depression comprises treatment resistant depression.

As used herein, “anxiety” refers to anxiety disorders including generalized anxiety disorder, panic attacks, obsessive-compulsive disorders, phobias, and post-traumatic stress disorders. Symptoms include feelings of apprehension or dread or impending doom, feeling tense or jumpy, restlessness or irritability, difficulty controlling feelings of worry, anticipating the worst and being watchful for signs of danger, difficulty concentrating or mind going blank, and physical symptoms including heart palpitations, pounding or racing heart, shortness of breath, sweating, tremors or shaking, muscle tension, headaches, fatigue, insomnia, upset stomach, frequent urination, or diarrhea.

Described herein is a precision medicine approach for the classification and treatment of CNS disease in a subject. According to one aspect of the present disclosure, the method comprises, consists of, or consists essentially of one or more of the following: (i) Stratifying subjects afflicted with a CNS disease based on their mitochondrial related metabolic profiles, metabolites and ratios of metabolites that define unique metabolic conditions related to change in mitochondrial function and common identity among subgroups of subjects; (ii) following the trajectory of disease within each subgroup of subjects and their response to treatment using both biochemical and clinical approaches; (iii) highlighting defects in transport and/or biosynthesis breakdown of mitochondrial related metabolites within a pathway or across pathways using ratios of metabolites to inform about changes in enzyme activities or transporters; (iv) looking for genetic basis of metabolic profile characteristic (SNPs/genetic variants in key enzymes and transporters) mGWAS analysis; (v) using combined metabotype and genotype data to better stratify patients with neuropsychiatric diseases and to inform about mechanisms; (vi) suggesting therapeutic approach to correct metabolic defects in profile in subgroups of patients; (vii) comparing and contrast to metabolic defects noted in inborn errors of metabolism and use knowledge gained in treatment of inborn errors of metabolism to inform treatment for CNS diseases; (vii) administering to the subject a therapeutically effective amount of therapeutic intervention to prevent and/or treat the CNS disease; and (viii) monitoring metabolic changes during treatment to define and characterize individual treatment response.

Described herein is the discovery of a precision medicine approach for the classification and treatment of CNS disease in a subject. According to one aspect of the present disclosure, the method comprises, consists of, or consists essentially of one or more of the following: (i) Stratifying subjects afflicted with a CNS disease based on their metabolic profiles, metabolites and ratios of metabolites that define unique metabolic conditions related to change in mitochondrial function and common identity among subgroups of subjects; (ii) following trajectory of disease within each subgroup of subjects and their response to treatment; (iii) highlighting defects in transport and/or biosynthesis breakdown of metabolites within a pathway or across pathways using ratios of metabolites to inform about changes in enzyme activities or transporters implicated in mitochondrial function; (iv) identifying genetic basis of metabolic profile characteristic (SNPs/genetic variants in key enzymes and transporters) mGWAS analysis; (v) using combined metabotype and genotype data to better stratify subjects with a CNS disease and to inform about mechanisms; (vi) selecting therapies to correct metabolic defects noted in subgroups of subjects with specific mitochondrial dysfunction; (vii) selecting compounds for therapy based on metabolic profile defects noted in each subgroup of subjects; (viii) informing treatment selecting based on metabotype and possibly genotype; (ix) Selecting from available compounds that modulate mitochondrial energetics as possible drugs to be repurposed for treatment of neuropsychiatric diseases; (x) using combinations of compounds, substrates substrate analogs, and co-factors that can improve mitochondrial function, the compounds used alone or in combination with other therapies currently used for treatment of CNS disease; (xi) comparing and contrasting to metabolic defects noted in inborn errors of metabolism related to mitochondrial dysfunction and using the knowledge gained in treatment of inborn errors of metabolism to inform treatment for CNS diseases; (xii) genetically screening for inborn errors of metabolism in subjects with a neuropsychiatric disease or subjects at risk of developing a neuropsychiatric disease; (xiii) using the metabolic profile as outcome to see if metabolic correction is achieved; (xiv) tailoring therapies that can ameliorate clinical symptoms (e.g., such as fatigue, sleep perturbation anxiety appetite changes motor function) and use of the selected compounds to correct energy metabolism as modalities to improve these symptoms; and (xv) repeating any one of steps (i)-(xv) to address and correct for metabolic defects related to neuropsychiatric and neurodegenerative symptoms across the CNS disease.

In some embodiments, the method further comprises identifying, testing, and characterizing available compounds that modulate mitochondrial energetics as possible drugs to be repurposed for treatment of subject suffering from a neurodegenerative disease. In other embodiments, the method further comprises identifying, testing, and characterizing possible drug combinations that modulate mitochondrial energetics as possible drugs to be repurposed for treatment of subject suffering from a CNS disease.

In another embodiments the method further comprises a control sample, the control sample comprises a mitochondrial metabolic profile comprising a plurality of stratified subjects afflicted with a CNS disease based on their mitochondrial metabolic profiles, mitochondrial metabolites, and ratios of mitochondrial metabolites that define unique mitochondrial metabolic conditions that identify for common identities among subgroups of subjects such as patients with treatment resistant depression.

In another embodiment, the therapeutic intervention comprises administering to the subject a therapeutically effective amount of a compound selected from peroxisome proliferator-activated receptor (PPAR) agonists, PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acid: Even (C₆-C₁₂), medium chain fatty acid: Odd chain fatty acids (C7, C₉), branched chain amino acids, carnitine, acetyl carnitine and short chain acylcarnitines (C₂₋₅), medium chain acylcarnitines and analogs, cofactors NAD Flavin FAD, and combinations thereof.

In other embodiments, the therapeutic intervention comprises the use of a combination of compounds within this class of therapies and or in combination with therapies currently used for treatment of CNS disease such as SSRIs or ketamine esketamine for treatment of depression or compounds being developed for treating resistant depression. In some embodiments, the combination of selected compounds is based on metabolic profile defects noted in each subgroup of subjects (e.g., metabotype and genotype to inform about treatment selection).

In another embodiment, the therapy comprises a drug or drug combination selected from one or more of carnitine and structural analogs or L-acetyl carnitine and structural analogs, short chain (C₂-C₅) acetyl L-carnitines, medium chain acylcarnitines, and analogs thereof, PPARα agonists, PPARγ agonists, PPARδ agonists, PPAR dual agonists, PPAR pan agonists, metformin, Triheptanoin, ketone bodies, short chain fatty acids, medium chain fatty acids, even chain (C₆-C₁₂) fatty acids, odd chain (C₇, C₉) fatty acids, branched chain amino acids, cofactors (e.g., NAD, riboflavin, FAD, Q10), or combinations thereof. Of special interest are combinations of cannabinoids and cannabinoid-like compounds (e.g., PEA) and carnitine or L-acetyl carnitines and structural analogs.

Studies have shown that C12 (dodecanoylcarnitine) levels are low in depression and particularly treatment resistant depression. Therapies or supplements to adjust the levels of C12 (dodecanoylcarnitine) may be useful in treating treatment resistant depression. In addition, octanoyltransferase, which generates C12 (dodecanoylcarnitine), make this enzyme a possible mechanistic target for therapeutics targeting depression and treatment resistant depression.

Another embodiment, described herein is a method for identifying and treating a subject suffering from, or at risk of suffering from, a CNS disease comprising, consisting of, or consisting essentially of one or more of the following steps: (i) stratifying subjects afflicted with a CNS disease based on their metabolic profiles, metabolites and ratios of metabolites that define unique metabolic conditions that identify for each and common identities among subgroups of subjects; (ii) following the trajectory of disease (biochemical and clinical) within each subgroup of subjects and their response to treatment; (iii) highlighting defects in transport and/or biosynthesis breakdown of mitochondrial related metabolites within a pathway or across pathways using ratios of mitochondria related metabolites to inform about changes in enzyme or enzyme activities; (iv) identifying genetic basis of metabolic profile characteristic (SNPs/genetic variants in key enzymes and transporters) mGWAS analysis; (v) using combined metabotype and genotype data to better stratify subjects with neuropsychiatric diseases and to inform about mechanisms; (vi) suggesting therapeutic approach to correct metabolic defects in profile in subgroups of subjects; (vii) comparing and contrast to metabolic defects noted in inborn errors of metabolism and use knowledge gained in treatment of inborn errors of metabolism to inform treatment for CNS diseases; (viii) genetically screening for inborn errors of metabolism in subjects with a neuropsychiatric disease or subjects at risk of developing a neuropsychiatric disease; and (ix) administering to the subject an effective amount of a therapy or recommending a life-style change intervention (based on inborn errors of metabolism) to prevent and/or treat the CNS disease.

In some embodiments, the methods further provide for the testing of available compounds that modulate mitochondrial energetics for use as possible drugs to be repurposed for treatment of the CNS disease(s).

In another embodiment, the method further provides for the use of the metabolic profile as an outcome to see if metabolic correction is achieved.

In another embodiment, the methods further provide for the evaluation of clinical symptoms in heterogenous diseases (e.g., like depression, anxiety, etc.) by using metabolic profile to inform about basis of clinical symptoms (e.g., such a fatigue, sleep perturbation anxiety appetite changes motor function), identifying compounds that correct energy metabolism as modalities to improve these symptoms, and treating the subject with the identified compounds.

In another embodiment, the methods further provide for the identification of metabolic defects and treatment of said metabolic defects related to a CNS disease.

Pathways and metabolic reactions within mitochondria to which the methods provided herein can be applied include acylcarnitine biosynthesis/transport, fatty acid transport and beta oxidation, interconnected TCA and urea cycles and their links to glycolysis, production of ketone bodies, acetyl CoA homeostasis, and utilization of short chain fatty acids and branched chain amino acids (BCAA), among others.

Another embodiment described herein is the utilization of the results of a comprehensive analysis of plasma and CSF lipid mediators in a case-control comparison of patients with Alzheimer's disease, with a targeted quantitative mass spectrometry approach. In both plasma and CSF, Alzheimer's disease patients were observed to have elevated components of cytochrome P450/soluble epoxide hydrolase pathway and lower levels of fatty acids ethanolamides, when compared to the healthy controls. Multivariate analysis revealed that circulating metabolites of soluble epoxide hydrolase together with ethanolamides are strong and independent predictors for Alzheimer's disease and cognitive dysfunction. Both metabolic pathways are potent regulators of inflammation with soluble epoxide hydrolase being reported to be upregulated in the brains of Alzheimer's disease patients. This study provides further evidence for the involvement of inflammation in Alzheimer's disease and argues for further research into the role of the cytochrome P450/soluble epoxide hydrolase pathway and fatty acid ethanolamides in this disorder. Ethanolamides, their relationship to natural cannabinoid compounds, and the regulation of common receptors implicated in mitochondrial energetics, present new possibilities for developing drugs that can ameliorate CNS disease symptoms. Further, these findings suggest that a combined pharmacological intervention targeting both metabolic pathways may have therapeutic benefits for Alzheimer's disease.

### Acylcarnitine Pathway

Acylcarnitine homeostasis disruption and mitochondrial energy defects can occur under a variety of conditions due to the presence of various genetic variants and environmental changes. Using metabolomics and lipidomics approaches in combination with genomics, each of these conditions can be precisely subtyped by measuring metabolites involved in energy metabolism and by calculating their ratios. For example, measurement of carnitine and acylcarnitines, and their ratios in combination with other metabolomics/lipidomics measurement can address inquiries such as: (1) Which types of energy substrates are used abnormally?; (2) Which stage of fatty acid oxidation has been disrupted?; (3) Is there a problem with carnitine biosynthesis and/or uptake?; (4) Are there problems with carnitine or fatty acid transport?; (5) Is there deficient BCAA that affects their link to short chain acylcarnitines?; (6) Are there chronic disorder(s) within the microbiome that produce short chain fatty acids related to short chain acyl carnitines?; (7) Are there problems in short-, medium-, and long-chain acyl-CoA dehydrogenase enzymes including ACADS, ACADM, and ACADL that are involved in beta-oxidation, and is there a genetic link or links to inborn errors of metabolism?; and (8) Is there a problem in Electron transfer flavoprotein-ubiquinone oxidoreductase (ETFDH)? The ETFDH gene encodes a component of the electron-transfer system in mitochondria and is essential for electron transfer from several mitochondrial flavin-containing dehydrogenases to the main respiratory chain. Mutations in the ETFDH gene can cause glutaric aciduria 2C (GA2C), an autosomal recessively-inherited disorder of fatty acid, amino acid, and choline metabolism. It is characterized by multiple acyl-CoA dehydrogenase deficiencies resulting in large excretion not only of glutaric acid, but also of lactic, ethylmalonic, butyric, isobutyric, 2-methyl-butyric, and isovaleric acids.

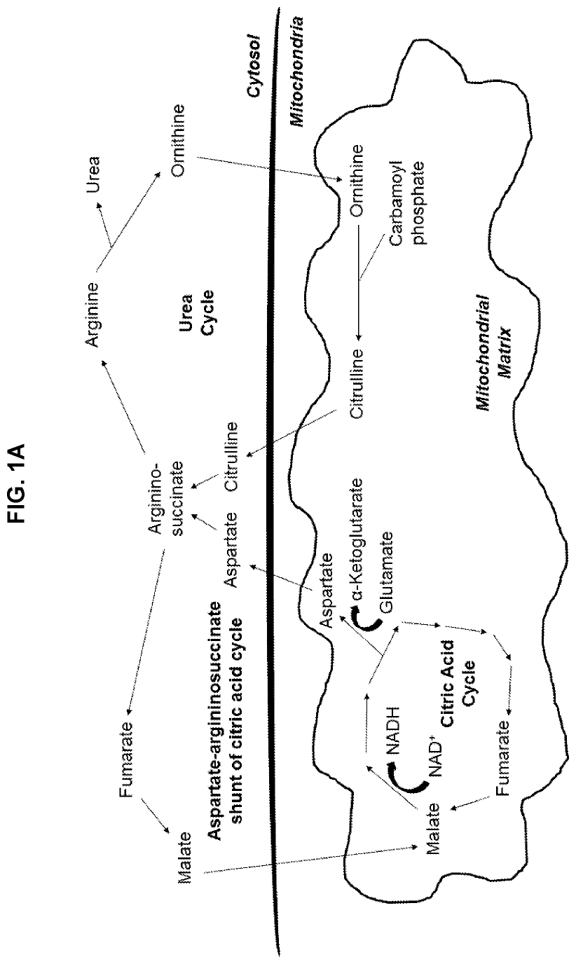

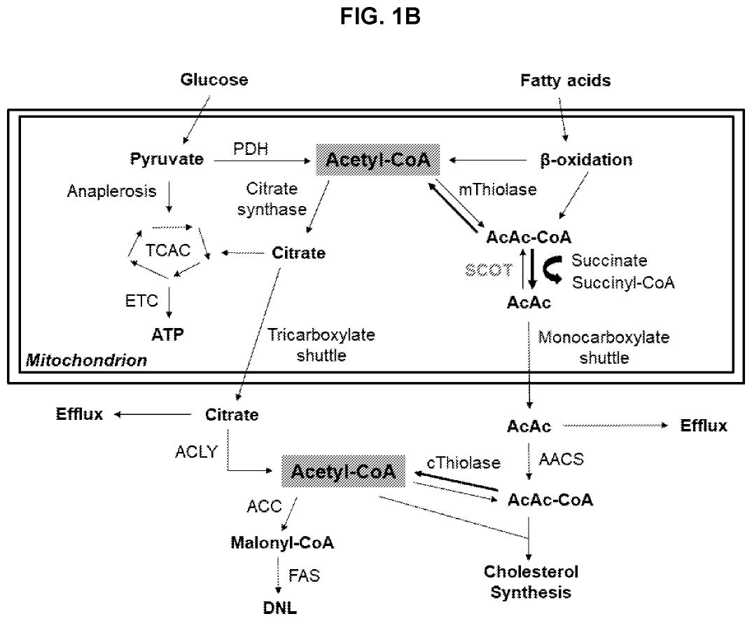

General mechanistic schemes of the mitochondrial energetics contribution of glycolysis, fatty acid oxidation, amines, energy production through the citric acid cycle (TCA) and linked urea cycle, electron respiratory chain (ETC), as well as connections to de novo fatty acid synthesis, cholesterol synthesis, and ketone body production, are shown in part in FIG. 1A-B.

The other metabolites used for subtyping acylcarnitine homeostasis disruption and mitochondrial energy defects involve a variety of fatty acids (short to long chains), triacylglycerol, citric acid and other intermediates of TCA cycle, the contribution of glycolysis to the TCA cycle and other organic acids, dicarboxylic acids, amino acids (particularly branched chain amino acids (BCAA)). Described herein are examples involving disruption of acylcarnitine homeostasis, including the possible causal factors, approach for identification, and potential treatments.

Pathways/genes are involved in carnitine/acylcarnitine metabolism and transport, thereby affecting their homeostasis peripherally as well as energy homeostasis in the brain, and subsequently contribute to depression and/or other neuropsychiatric and neurodegenerative disorders. The altered pathways/gene variants could be categorized into three scenarios (Scenarios 1-3):

### Scenario 1

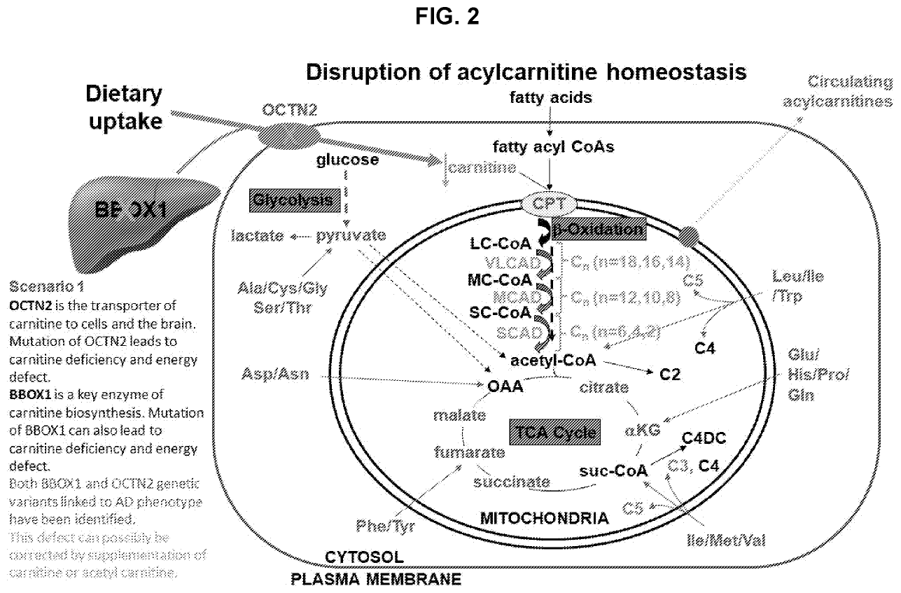

Scenario 1 relates to carnitine biosynthesis and transport to cells and to the brain. The gene associated with the key step of carnitine de novo synthesis is BBOX1. The variants of BBOX1 results in systemic deficiency in carnitine and thus acylcarnitines. The main gene associated with carnitine transport to many cell types and the blood brain barrier (BBB) to the brain is OCTN2. The presence of OCTN2 variants leads to higher or normal levels of free carnitine, but deficiency in systemic acylcarnitines. The possible association of this scenario with psychiatric disorders can be metabotyped with the reduced levels of all kinds of acylcarnitine species and conformed with the presence of these gene variants from genotyping. The metabolic difference between two cases is the free carnitine levels in the circulation system (see FIG. 2).

Once this scenario is identified by genotyping and metabotyping, treatment of this scenario can be achieved by supplementing acetylcarnitine, propionylcarnitine or other short chain acylcarnitines, as well as synthetic analogs of carnitines. This is due to the fact that the transport of these short chain acylcarnitines is less dependent on OCTN2, but likely on OCTN1. Moreover, Supplementation of any kinds of carnitine-independent energy substrates such as oils enriched with medium chain fatty acids, short-chain fatty acids (e.g., propionic acid), or ketone bodies in combination with glucose and amino acids should be useful for maintaining energy homeostasis under this scenario.

### Scenario 2

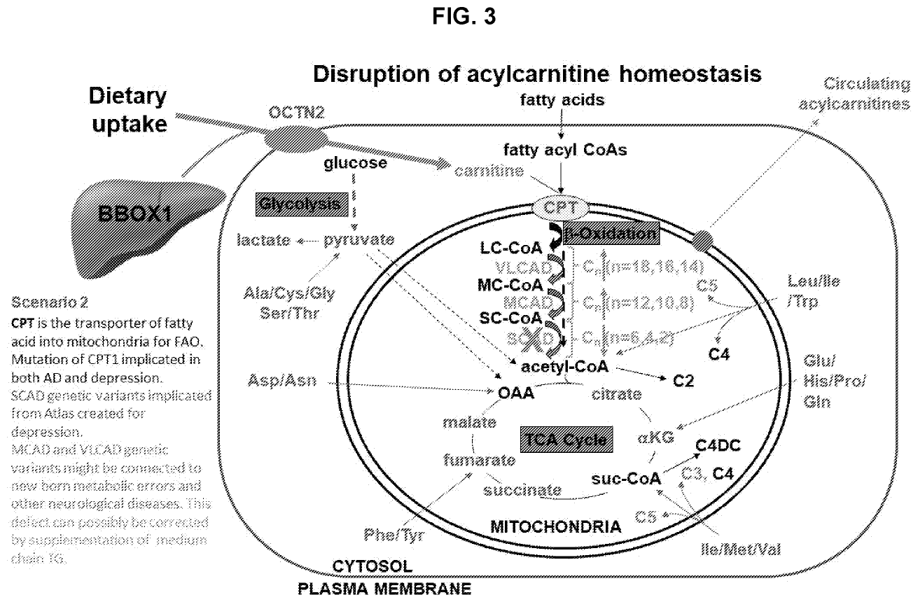

Scenario 2 is related to long chain fatty acid (FA) as an energy substrate including FA transport and oxidation. The genes involving this category mainly include the carnitine palmitoyltransferase (CPT) machinery (CPT1, CPT2, or CACT), (very) long chain acylCoA dehydrogenase (V/LCAD), medium chain acylCoA dehydrogenase (MCAD), and short chain acylCoA dehydrogenase (SCAD). Severe losses of protein/enzyme functions due to some variants of these genes are associated with inborn error diseases. Mild losses of the functions of the gene variants lead to energy deficiency. Specifically, CPT variants lead to a similar phenotype of OCTN2 variants as described above, i.e., greater, or normal levels of free carnitine, but deficiency in systemic acylcarnitines; V/LCAD variants lead to accumulation of long-chain acylcarnitines, but deficiency in medium and short chain acylcarnitines. Both MCAD and SCAD variants lead to accumulation of long and medium-chain acylcarnitines, but deficiency in short chain acylcarnitines. The image on the left shows the case of SCAD defects which result in accumulation of long and medium chain acylcarnitine, but deficiency in short chain acylcarnitines, particularly those of odd-numbered short chain acylcarnitines as the precursors of these odd-numbered short chain acylcarnitines are key substrates for synthesis of TCA cycle intermediates (see FIG. 3).

The following are examples of the metabotyping methods for determining these defects: CPT defects, lower levels of all acylcarnitines and higher ratios of carnitine/C3:0, carnitine/C5:0, carnitine/C10:0, carnitine/C16:0, and carnitine/C18:1 acylcarnitines; V/LCAD defects, lower ratios of short and medium chain vs. long chain acylcarnitines (including C3:0/C16:0, C5:0/C16:0, C10:0/C16:0, C3:0/C18:1, C5:0/C18:1, and C10:0/C18:1); MCAD defects, lower ratios of short chain vs. medium and long chain acylcarnitines (including C3:0/C10:0, C5:0/C10:0, C3:0/C16:0, C5:0/C16:0, C3:0/C18:1 and C5:0/C18:1); SCAD defects, lower ratios of odd-numbered short chain acylcarnitines vs. even-numbered short chain acylcarnitines (e.g., C6), medium and long chain acylcarnitines (including C3:0/C6:0, C5:0/C6:0, C3:0/C10:0, C5:0/C10:0, C3:0/C16:0, C5:0/C16:0, C3:0/C18:1 and C5:0/C18:1). Furthermore, mGWAS analysis can be conducted on mitochondrial metabolites and ratios of mitochondrial metabolites to inform about genetic basis of metabolic changes as shown in the examples below.

Once this scenario is identified by genotyping and metabotyping, treatment of this scenario can be achieved by supplementing substrates such as oils enriched with medium chain fatty acids, short-chain fatty acids (e.g., propionic acid), or ketone bodies in combination with glucose and amino acids, particularly BCAA.

### Scenario 3

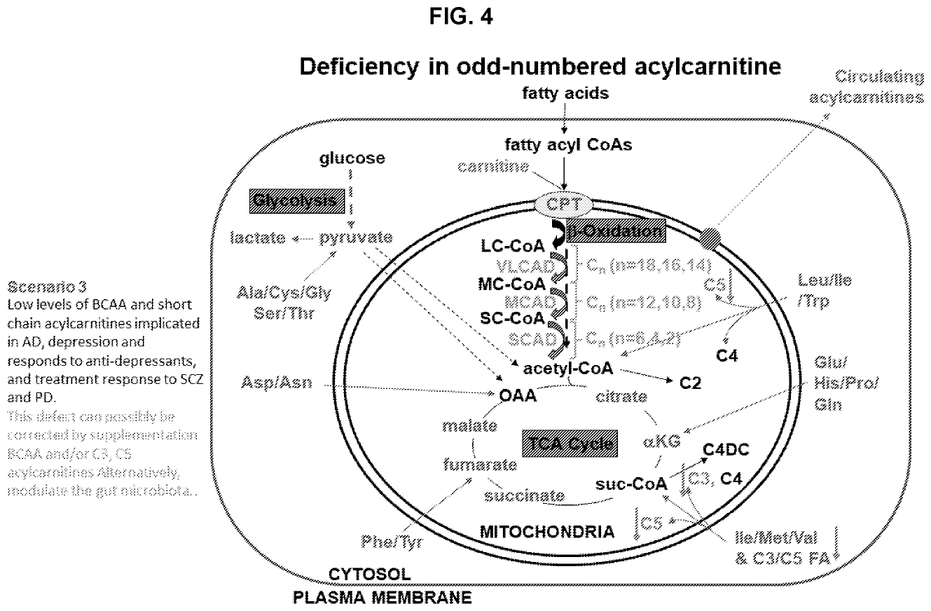

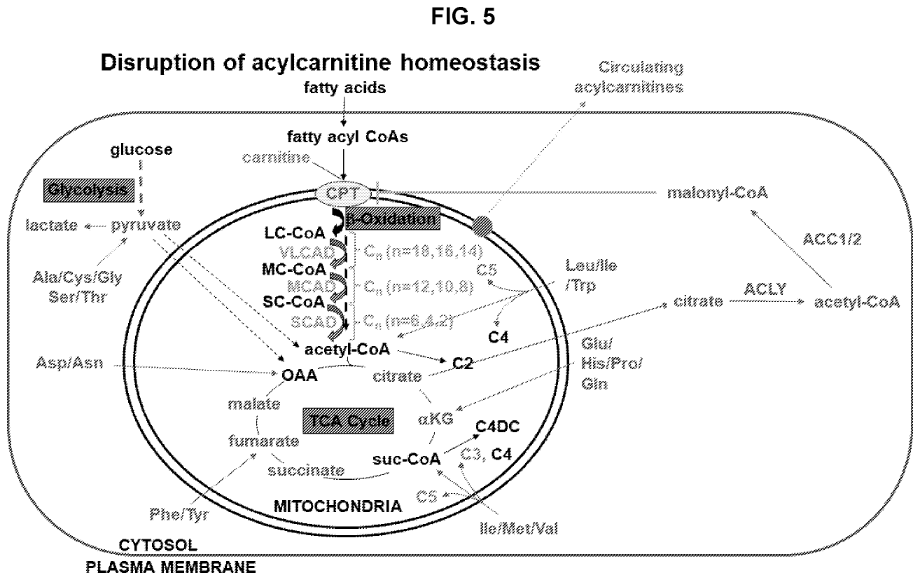

Scenario 3 is related to the deficiency in amino acids (e.g., BCAA) and/or short chain fatty acids, likely due to diabetes and gut microbiome disorder. This scenario can be detected with the reduced levels of C₃ and C₅ acylcarnitines while the levels of other acylcarnitines are virtually normal. Supplementing BCAA, C₃/C₅ acylcarnitines and derivatives and structural analogs, propionic acid, ketone bodies, etc. should be useful for correction of this scenario (see FIG. 4-5).

There exist many other pathways/genes related to carnitine metabolism and homeostasis. A genotyping/metabotyping method can be similarly developed to detect these subtypes of cases which can be treated with those outlined above (i.e., inhibitors combining with supplementation of different energy substrates). Overall, in many cases, supplementing with carnitine, acetyl carnitine, propionyl carnitine, or other precursors/ester derivatives/structural analogs and/or short-/medium-chain fatty acid should be a useful therapy. For example, the variants of genes (i.e., ALCY and ACC1/2) carrying the enzymes leading to malonyl-CoA synthesis do not directly affect the acylcarnitine homeostasis, however, since malonyl-CoA is a feed-up inhibitor of CTP1 which transports long chain fatty acids into mitochondria for oxidation, defects in malonyl-CoA synthesis could lead to over fluxing fatty acids into mitochondria and cause mitochondrial dysfunction. This type of case can be detected from the accumulation of all types of acylcarnitines in combination with GWAS analysis of ALCY and ACC1/2 variants. Targeting inhibition of CPT1 could be used to treat this type of aberration. Any defects in genes and/or proteins involving TCA cycle have high impacts on acylcarnitine homeostasis. Both PPARα and PPARγ are also associated with acylcarnitine homeostasis, the former is related to mitochondrial biogenesis and the latter is related to peroxisomal proliferation (activation of peroxisome could modulate the availability for medium chain fatty acids. Collectively, a list of genes which may have connections to acylcarnitine homeostasis and CNS diseases is given below.

In other embodiments, the methods described herein can be applied to other pathways and metabolic reactions within mitochondria that contribute to energy production and its homeostasis such as interconnected TCA and urea cycles, production of ketone bodies from acetyl CoA, utilization of short chain fatty acids and BCAA, link of glycolysis to acetyl CoA and TCA cycle among others that can lead to a roadmap for defining energy metabolic defects in sub groups of subjects and at an individual level where genotype and metabotype inform about source of metabolic defect in a subject and the tailoring of supplements, diet recommendations and treatments optimized based on knowledge gained about the subject. If similar defects have been noted in inborn errors of metabolism at severe levels other therapeutic approaches can be used and will be informed by improving subject's inborn errors of metabolism.

The methods provided herein allow for the identification and characterization of variants implicated in regulation of mitochondrial energy homeostasis. For example, in studies of acylcarnitine defects, one can focus on the genes listed below, among others, and investigate whether the genetic variants in these genes are implicated in CNS diseases or symptoms and how this might correlate with an altered metabolic profile measured in blood or other body fluids.

TABLE 1
Possible Gene Variants and Links to CNS Diseases

| Gene | Link to CNS Diseases |
|---|---|
| BBOX1 | Carnitine synthesis |
| OCTN2 | Carnitine transport |
| CPT1/CPT2 | LCFA transport into mitochondria |
| CACT | LCFA transport into mitochondria |
| All TCA cycle | TCA cycle metabolism and regulation and |
| genes | mitochondrial energetics |
| Ketogenesis | Acetyl-CoA regulation and homeostasis |
| and glycolysis | |
| enzymes | |
| prpE | SCFA:CoA ligase (e.g., propanoate:CoA ligase |
| ACC1/ACC2 | Initiation of fatty acid synthesis |
| ACLY | ATP citrate lyase |
| PPARα/PPARγ | Acylcarnitine homeostasis and mitochondrial |
| | energetics |
| ACADS | Short-chain acyl-CoA dehydrogenase (beta oxidation) |
| ACADM | Medium-chain acyl-CoA dehydrogenase (beta |
| | oxidation) |
| ACADL | Long-chain acyl-CoA dehydrogenase (beta oxidation) |
| ETFDH | Electron transfer flavoprotein-ubiquinone |
| | oxidoreductase (defects can lead to GA2C disorder) |

In some embodiments, the list of genes is constructed for carnitine and acylcarnitine regulation and transport, TCA cycle, urea cycle, glycolysis regulation, ketone body production, BCAA synthesis and breakdown, short-chain acylcarnitines and links to acetyl CoA and malonyl CoA production, and acetyl CoA homeostasis.

Accordingly, numerous biomarkers may be used in the methods provided herein. In some embodiments, the biomarker metabolite comprises one or more short-chain Acylcarnitines. Examples include, but are not limited to, C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C₆-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); and/or Long-Chain Acylcarnitines selected from C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine), ketone bodies and/or acetyl CoA compounds.

In some embodiments, the control sample comprises a metabolic profile comprising a plurality of stratified subjects afflicted with a CNS disease based on their metabolic profiles, metabolites, and ratios of metabolites that define unique metabolic conditions that identify for common identities among subgroups of subjects.

In another embodiment, the therapeutic intervention comprises administering to the subject a therapeutically effective amount of a compound selected from one or more of PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acids: even (C₆-C₁₂), medium chain fatty acids; odd chain fatty acids (C₇, C₉), branched chain amino acids, NAD, riboflavin, FAD cofactors Q10, carnitine, acetyl carnitine, short chain acylcarnitines, or combinations thereof.

The compounds provided below are examples of compounds that can be repurposed for treatment of a CNS disease (e.g., depression and neuropsychiatric diseases and symptoms), to treat clinical symptoms in CNS diseases and clinical symptoms in CNS diseases, such as fatigue, anxiety, sleep perturbation, appetite changes, and motor changes. The compounds can be used alone or as a combination with other drugs, compounds, therapies, etc. used currently to treat said CNS diseases, or they can be combined with drugs that improve mitochondrial energetics (e.g., cofactors NAD Flavins and carnitine/L-acetyl carnitine, PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acids: even (C₆-C₁₂), medium chain fatty acids; odd chain fatty acids (C7, C₉), branched chain amino acids, NAD, riboflavin, FAD cofactors Q10, carnitine, acetyl carnitine, short chain acylcarnitines, or combinations thereof.

In some embodiments, the compounds are to be used alone or in combination and along with drugs currently used for treatment of CNS diseases. Metabolic signatures of these drugs can be defined using approaches developed by the inventors utilizing pharmacometabolomics where metabolic profiles before and after drug treatment provides information on pathways drugs affected.

In some embodiments, these pathways are tested to see if they correct for metabolic defects seen in subjects. Based on the results, a personalized selection of drugs based on metabotype genotype of the subject and the metabolic imprint of the drug of choice.

The compounds provided below are provided as examples only and are not intended limiting in any way.

### PPARs

### PPARα Agonists

Peroxisome proliferator-activated receptor-alpha (PPARα) is a nuclear receptor activated by ligand binding. Endogenous ligands for the receptor include fatty acids such as arachidonic acid, polyunsaturated fatty acids, and some fatty acid-derived compounds, Palmitoylethanolamide (PEA), a naturally occurring amide of ethanolamine and palmitic acid with anti-inflammatory and endocannabinoid effects, ethanolamides and cannabinoids.

Expression of PPARα is highest in tissues that that have a high rate of fatty acid oxidation such as the liver. PPARα is master regulator of lipid metabolism that impacts bile synthesis/secretion, fatty acid uptake, mitochondrial β-oxidation and peroxisomal fatty acid oxidation, and ketogenesis.

Fibrates are a class of amphipathic carboxylic acids used in the treatment of dyslipidemia lowering serum triglycerides and LDL and raising serum HDL-cholesterol levels. Fibrates (simfibrate, clofibrate, gemfibrozil, ciprofibrate, bezafibrate, and fenofibrate) are exogenous ligands for the PPARα receptor. Their ability to reduce triglycerides and increase HDL has tentatively been linked an ability to reduce insulin resistance in subjects with metabolic syndrome or diabetes. Multiple mechanisms for the reduction in insulin resistance have been hypothesized, including regulation of hepatic TRB-3 and lowering of intracellular lipids. In addition, many studies have found that fibrates may reduce inflammation. It is possible that the reduction in chronic inflammation may reduce insulin resistance.

PPARα is localized in brain regions involved in the regulation of emotions and the stress response. Activation of PPARα may be a natural response to stress, with PPARα activation having the ability to mediate and modulate the stress response. This may contribute to PPARα activation effects as a mood stabilizer. Fibrates, or endogenous PPARα ligands, have been shown to act as anti-depressants in animal models and in human trials. These effects have been hypothesized to be from changes in brain cholesterol, regulation allopregnanolone biogenesis, reduction on neuroinflammation, and increased insulin sensitivity.

There are other classes of PPARα agonists including cannabinoids. Cannabinoids have been used medicinally and recreationally for thousands of years and their effects were proposed to occur mainly via activation of the G-protein-coupled receptor CB1/CB2 (cannabinoid receptor 1/2). Discovery of potent synthetic analogs of the natural cannabinoids as clinically useful drugs is the sustained aim of cannabinoid research.

In one embodiment, the cannabinoid PPARα agonist comprises a compound that can be isolated from Cannabis, derived from a natural cannabinoid (semi-synthetic), or chemically synthesized. In one embodiment, the cannabinoid comprises a compound having the structure of:

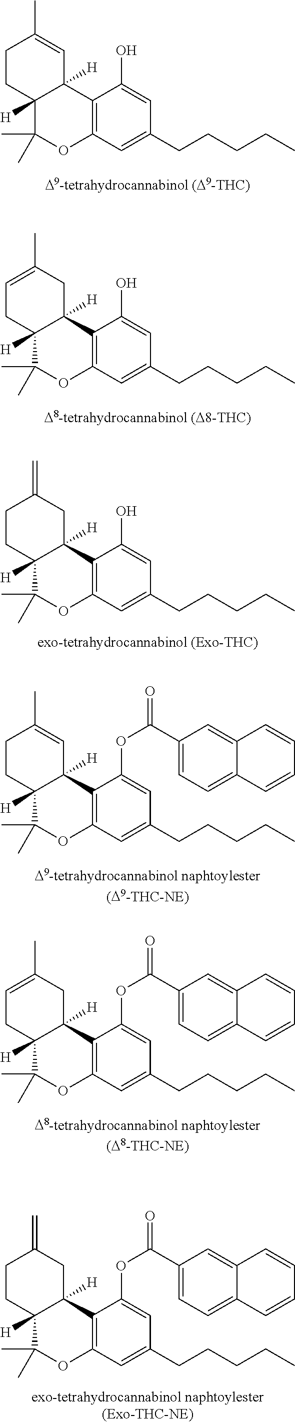

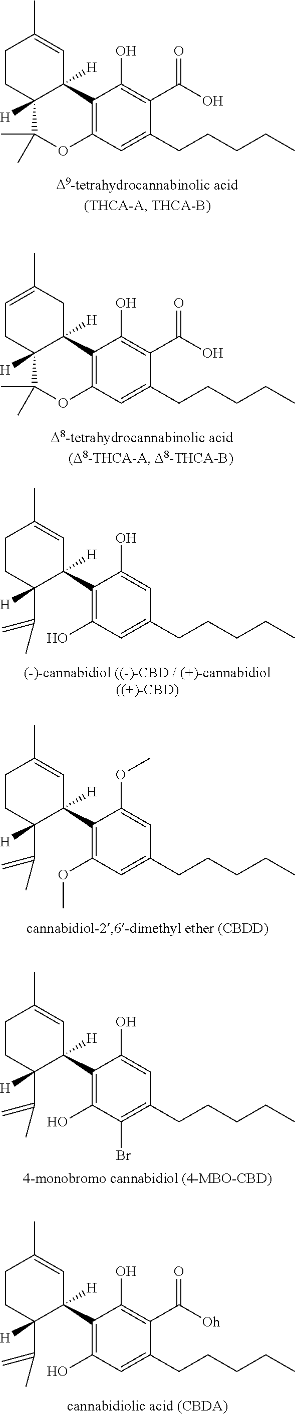

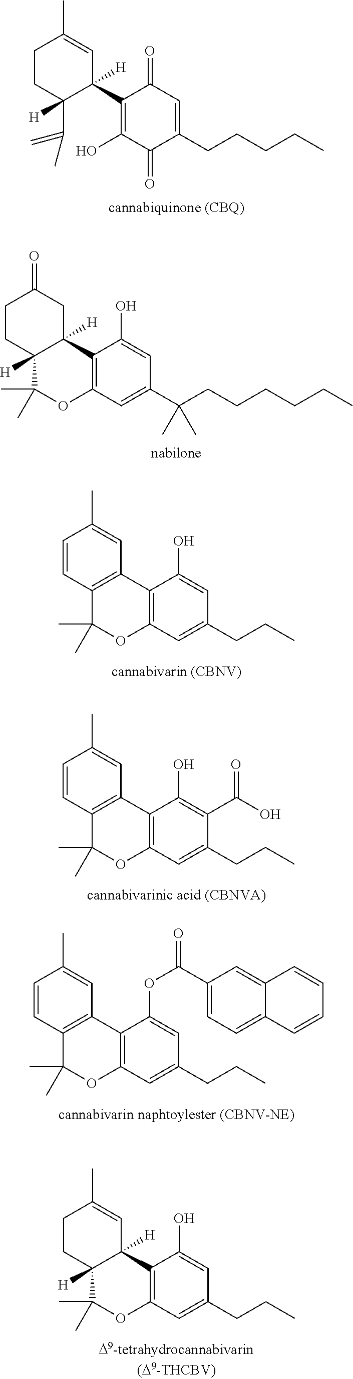

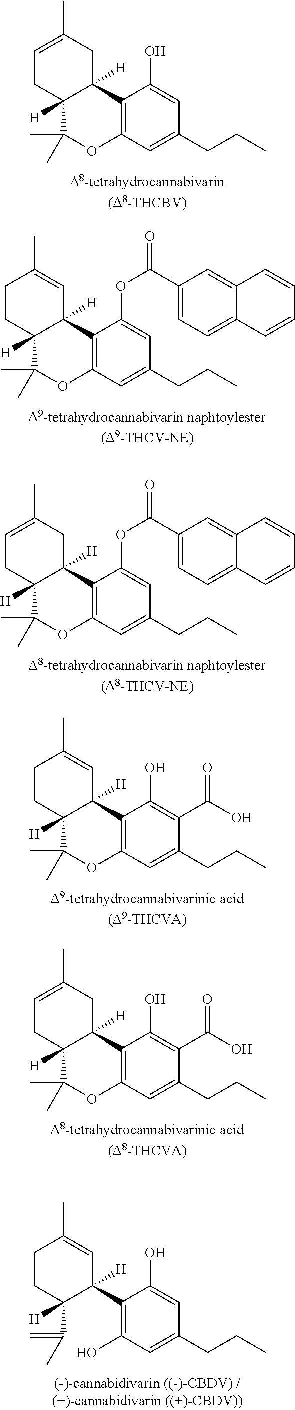

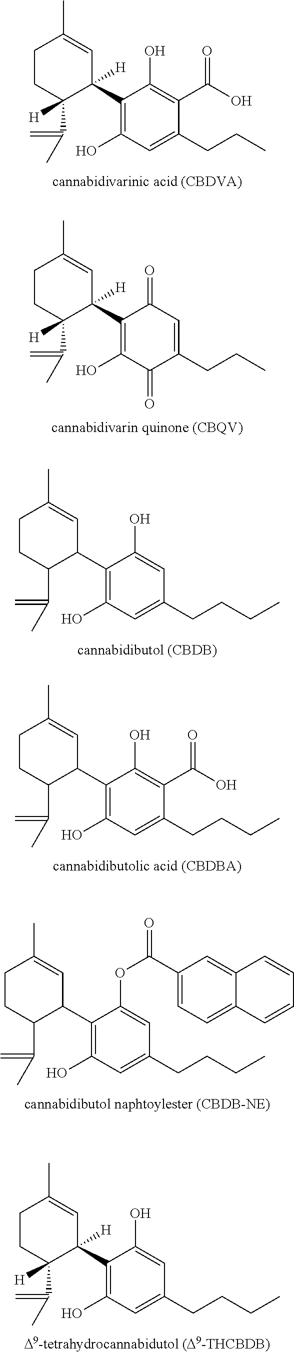

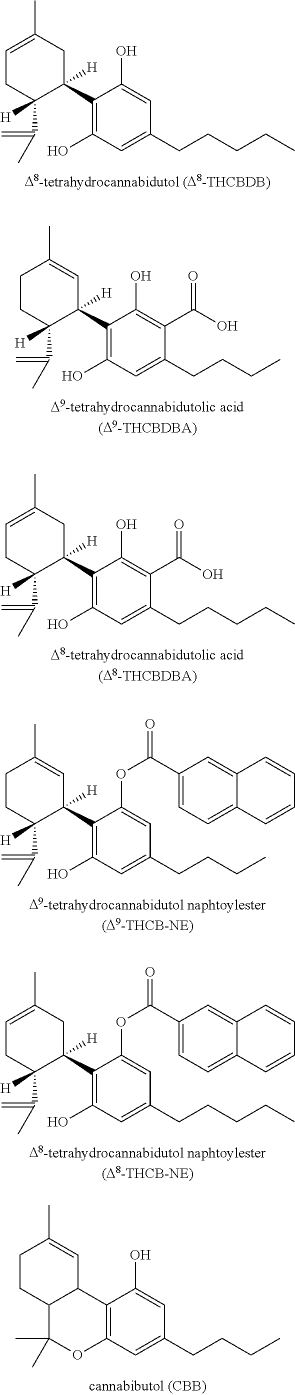

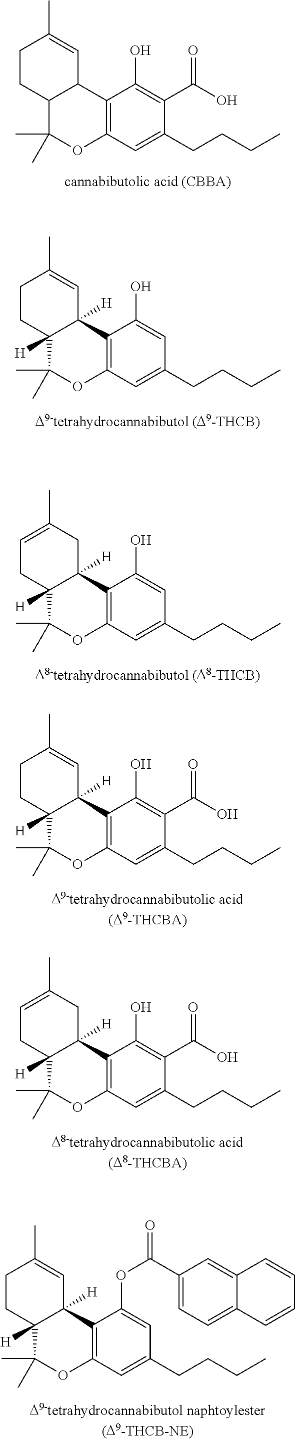

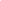

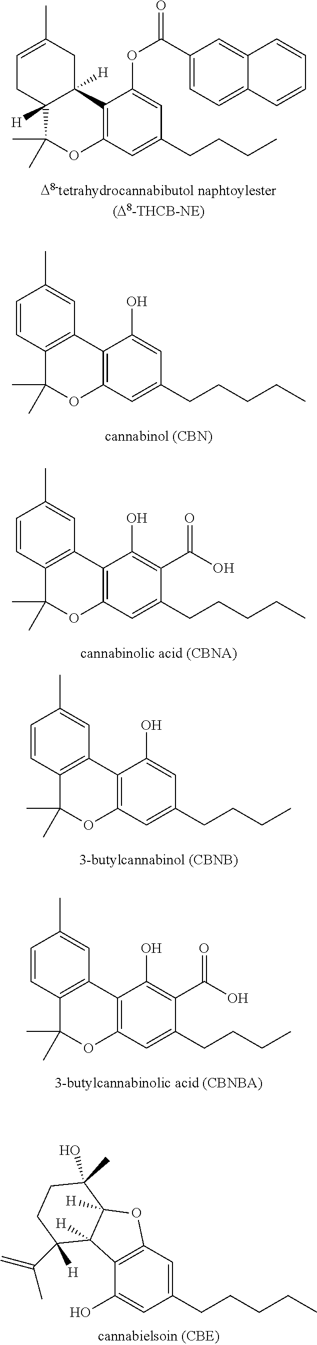

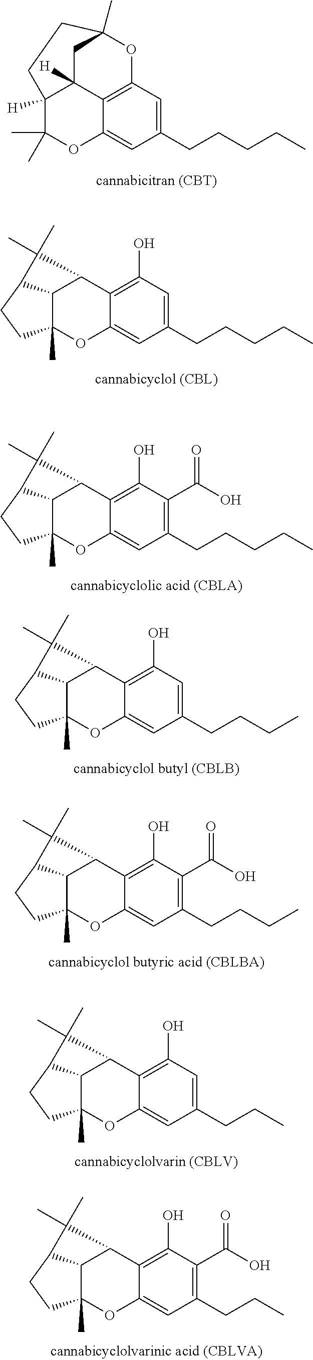

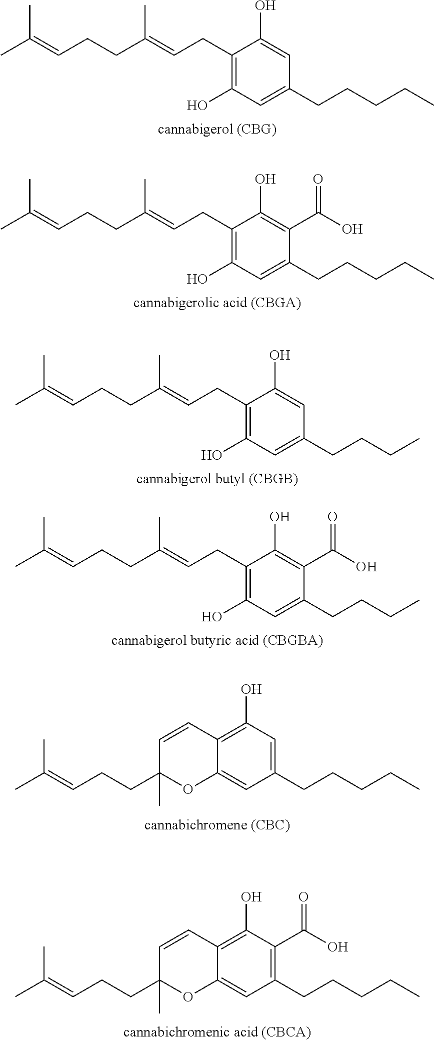

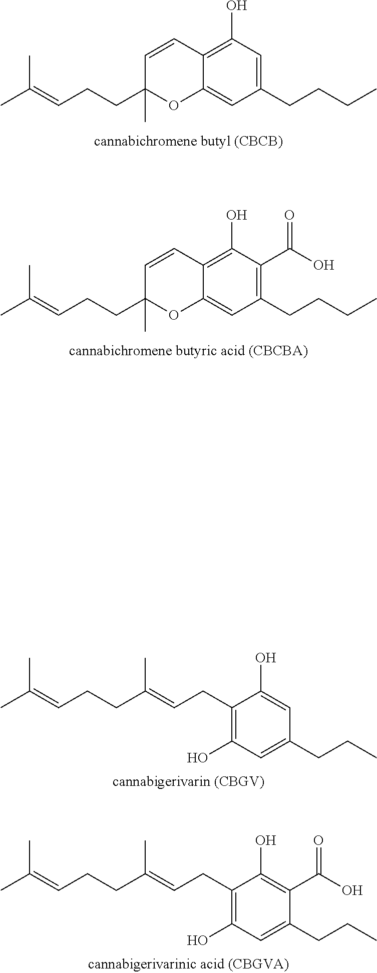

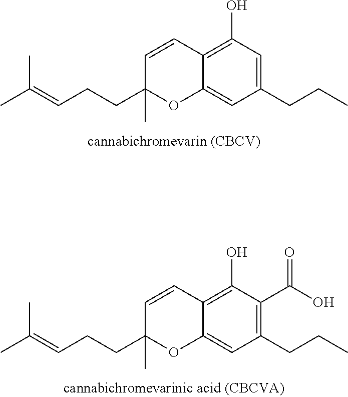

In another embodiment, the cannabinoid PPARα agonist comprises one or more of: Δ⁹-tetrahydrocannabinol (Δ⁹-THC; Dronabinol); Δ⁸-tetrahydrocannabinol (Δ⁸-THC); exo-tetrahydrocannabinol (Exo-THC); Δ⁹-tetrahydrocannabinol naphtoylester (Δ⁹-THC-NE); Δ⁸-tetrahydrocannabinol naphtoylester (Δ⁸-THC-NE); exo-tetrahydrocannabinol naphtoylester (Exo-THC-NE); Δ⁹-tetrahydrocannabinolic acid (THCA-A, THCA-B); Δ⁸-tetrahydrocannabinolic acid (Δ⁸-THCA-A, Δ⁸-THCA-B); (−)-cannabidiol ((−)-CBD)/(+)-cannabidiol ((+)-CBD); cannabidiol-2′,6′-dimethyl ether (CBDD); 4-monobromo cannabidiol (4-MBO-CBD); cannabidiolic acid (CBDA); cannabiquinone (CBQ); nabilone; cannabivarin (CBNV); cannabivarinic acid (CBNVA); cannabivarin naphtoylester (CBNV-NE); Δ⁹-tetrahydrocannabivarin (Δ⁹-THCBV); tetrahydrocannabivarin (Δ⁸-THCBV); Δ⁹-tetrahydrocannabivarin naphtoylester (Δ⁹-THCV-NE); Δ⁸-tetrahydrocannabivarin naphtoylester (Δ⁸-THCV-NE); Δ⁹-tetrahydrocannabivarinic acid (Δ⁹-THCVA); Δ⁸-tetrahydrocannabivarinic acid (Δ⁸-THCVA); (−)-cannabidivarin ((−)-CBDV)/(+)-cannabidivarin ((+)-CBDV)); cannabidivarinic acid (CBDVA); cannabidivarin quinone (CBQV); cannabidibutol (CBDB); cannabidibutolic acid (CBDBA); cannabidibutol naphtoylester (CBDB-NE); Δ⁹-tetrahydrocannabidutol (Δ⁹-THCBDB); Δ⁸-tetrahydrocannabidutol (Δ⁸-THCBDB); Δ⁹-tetrahydrocannabidutolic acid (Δ⁹-THCBDBA); Δ⁸-tetrahydrocannabidutolic acid (Δ⁸-THCBDBA); Δ⁹-tetrahydrocannabidutol naphtoylester (Δ⁹-THCB-NE); Δ⁸-tetrahydrocannabidutol naphtoylester (Δ⁸-THCB-NE); cannabibutol (CBB); cannabibutolic acid (CBBA); Δ⁹-tetrahydrocannabibutol (Δ⁹-THCB); Δ⁸-tetrahydrocannabibutol (Δ⁸-THCB); tetrahydrocannabibutoic acid (Δ⁹-THCBA); Δ⁸-tetrahydrocannabibutolic acid (Δ⁸-THCBA); Δ⁹-tetrahydrocannabibutol naphtoylester (Δ⁹-THCB-NE); Δ⁸-tetrahydrocannabibutol naphtoylester (Δ⁸-THCB-NE); cannabinol (CBN); cannabinolic acid (CBNA); 3-butylcannabinol (CBNB); 3-butylcannabinolic acid (CBNBA); cannabielsoin (CBE); cannabicitran (CBT); cannabicyclol (CBL); cannabicyclolic acid (CBLA); cannabicyclol butyl (CBLB); cannabicyclol butyric acid (CBLBA); cannabicyclolvarin (CBLV); cannabicyclolvarinic acid (CBLVA); cannabigerol (CBG); cannabigerolic acid (CBGA); cannabigerol butyl (CBGB); cannabigerol butyric acid (CBGBA); cannabichromene (CBC); cannabichromenic acid (CBCA); cannabichromene butyl (CBCB); cannabichromene butyric acid (CBCBA); cannabigerivarin (CBGV); cannabigerivarinic acid (CBGVA); cannabichromevarin (CBCV); cannabichromevarinic acid (CBCVA); other cannabinoids, or pharmaceutically acceptable salts, acids, esters, amides, hydrates, solvates, prodrugs, isomers, stereoisomers, tautomers, derivatives thereof, or combinations thereof.

New cannabinoid PPARα compounds optimally should be free of the psychotropic effects associated with recreationally used cannabinoids (e.g., Δ⁹-THC). In preclinical studies cannabinoids displayed many of the characteristics of nonsteroidal anti-inflammatory drugs (NSAIDs) and it seems to be free of unwanted side effects. An increasing number of therapeutic actions of cannabinoid are being reported that do not appear to be mediated by either CB1 or CB2, and recently nuclear receptor superfamily PPARs (peroxisome-proliferator-activated receptors) have been suggested as the target of certain cannabinoids.

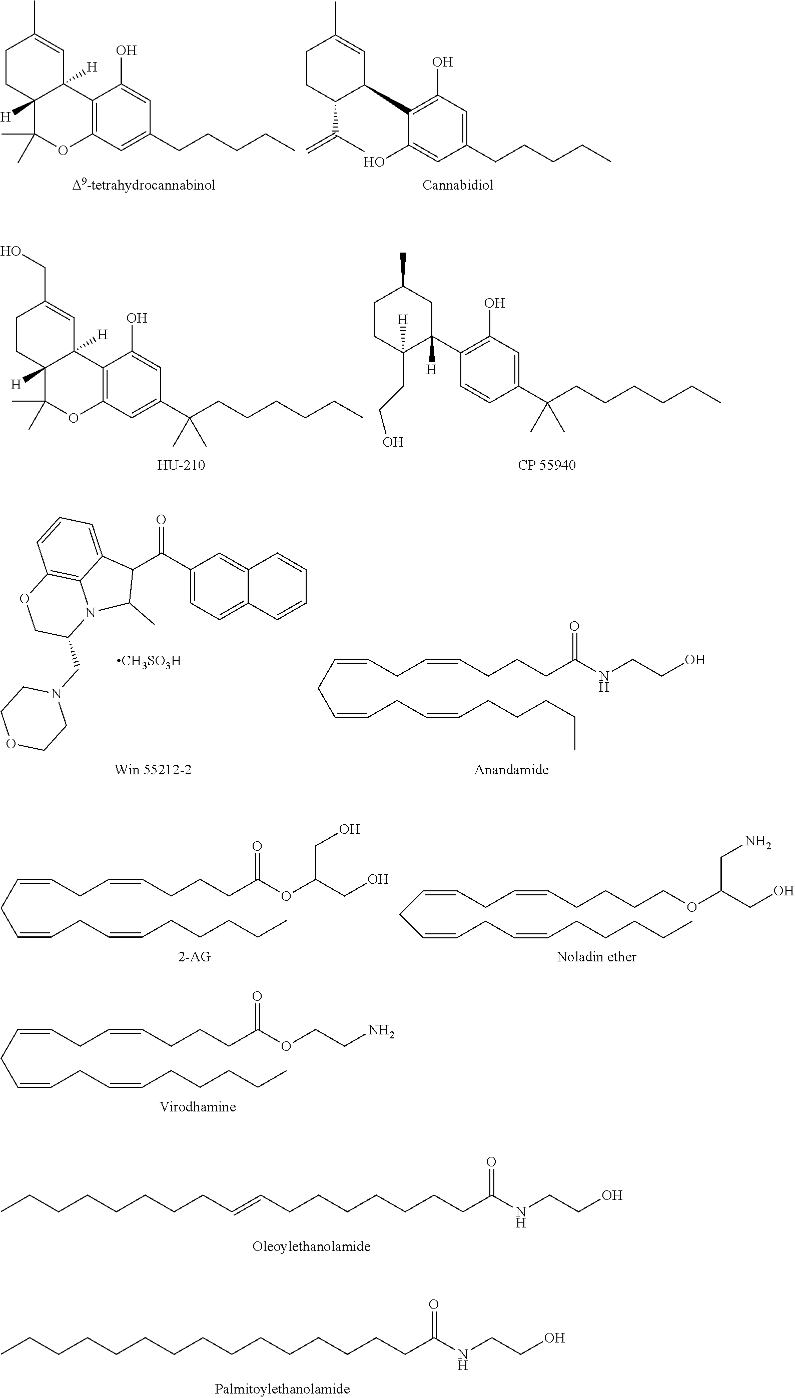

### PPARγ Agonists

PPARγ (PPAR-gamma) is a nuclear receptor and the main target of the drug class of thiazolidinediones (TZDs; pioglitazone and rosiglitazone). Thiazolidinediones are used to treat in diabetes mellitus and other diseases that feature insulin resistance and inflammation. Both agonists and antagonists of PPARγ have been developed. Endogenous ligands of the receptor include the unsaturated fatty acids, 5-hydroxyicosatetraenoicacids, and cannabinoids. In addition, the receptor is also activated by certain NSAIDs (such as ibuprofen or naproxen) and indoles.

While thiazolidinediones were originally developed as anti-diabetic drugs, they are now also used in treating hyperlipidemia in atherosclerosis where they function by increasing extra-hepatic cholesterol transport into HDL.

Thiazolidinedione activation of the PPARγ receptor triggers a signaling cascade and regulates multiple aspects of inflammation. Binding of PPARγ to coactivators appears to reduce the levels of coactivators available for binding to the pro-inflammatory transcription factor NF-kB. The reduction in active NF-kB causes a decrease in the expression of proinflammatory genes and an increase in polarization of macrophages to the anti-inflammatory M2 type. Animal studies have shown a possible role for thiazolidinediones in treatment of pulmonary inflammation and fibrosis, especially in asthma and COVID-19.

In addition to the reduction of peripheral inflammation, in the brain PPARγ inhibition of the NFκB pathway may mitigate inflammation and stimulating the Nrf2/ARE axis to neutralize oxidative stress. This effect is key in reducing long-term damage following CNS injury. The role of thiazolidinedione in reducing neural inflammation may also contribute to its function as an antidepressant. It is thought that both the central and peripheral anti-diabetic and anti-inflammatory effects of PPARγ agonists contribute to an improvement in cognitive function in neurodegenerative disorders such as AD.

### PPARβ/δ Agonists

PPAR-beta or PPAR-delta (PPARδ) is important in regulating fatty acid uptake, transport, and β-oxidation as well as insulin secretion and insulin sensitivity. PPARδ agonists are considered to be exercise mimetics which result in increased fat oxidation and uncoupling of oxidative phosphorylation. These functions are thought to protect against metabolic syndrome-related diseases. Endogenous ligands of PPARδ include oleic acid, arachidonic acid, and members of the 15-hydroxyicosatetraenoic acid family. None of the current exogenous PPARδ agonists have FDA approval as a drug. However, multiple structural types of agonists have been developed by several companies.

As with the other PPAR family members, PPARδ is also a regulator of inflammation. Studies have demonstrated that PPARδ encourages macrophages to change phenotype toward the alternative anti-inflammatory M2 type, thus increasing fatty acid metabolism and insulin sensitivity, and suppressing systemic inflammation. PPARδ has been shown to reduce stress-induced depressive symptoms in animal models as well as potentially lowering amyloid burden in AD models. The actions of PPARδ in the brain has been hypothesized to be the functional linking pathway between PPARγ and PPARα.

### Dual and Pan PPAR Agonists

A fourth class of PPAR agonists which bind to multiple PPAR isoforms, are currently under investigation for treatment of metabolic syndrome and inflammatory fibrosis (Table 2). The dual agonists include the experimental compounds elafibranor, aleglitazar, muraglitazar and tesaglitazar, and the newer pan compound IVA337. These agonists are being developed as treatments to reduce both hepatic steatosis and hepatic fibrosis from nonalcoholic steatohepatitis and other fibrotic diseases. Saroglitazar developed by Zydus Cadila is approved in India.

TABLE 2
Dual PPAR Agonists

| Dual PPARα/γ | Status/Indication/Comment |
|---|---|
| Chiglitazar (CS038) Hoffman | Phase II clinical trial; Type II |
| LaRoche | diabetes and metabolic disorders |
| AVE0847 (Shenzhen Chipscreen | Phase II clinical trails |
| Biosciences) | |
| Aleglitazar (R1439) | Phase III clinical trials; reduces |
| (GlaxoSmithKline) | TG and CVD, increase HDL-c |
| 5-substituted 2-benzoylamino- | Preclinical trials; Type II diabetes |
| benzoic acid derivatives (BVT-142) | |
| O-arylmandelic acid derivatives | Preclinical trials |
| Azaindole-α- | Preclinical trials |
| alkyloxyphenylpropionic acid | |
| Amide substituted/α-substituted β- | Preclinical trials |
| phenylpropionic acid derivatives | |
| 2-alkoxydihydrocinnamate | Preclinical trials; Type II diabetes |
| derivative | and atherosclerosis |
| α-aryloxy-α-methylhydrocinnamic | Preclinical trials; antidiabetic and |
| acids (LYS1029) | antilipidemic |
| TZD18 | Preclinical trials |
| α-aryloxyphenyl acetic acid | Preclinical trials |
| derivatives | |
| PLX249 | Preclinical trials; Type II diabetes |
| | and atherosclerosis |
| Muraglitazar, tesaglitazar, | Clinical trials discontinued for |
| naveglitazar, ragaglitazar, | safety issues (edema, weight gain, |
| farglitazar, imiglitazar, | cardiovascular risks, impairment |
| netoglitazone, compound 3q JTT- | of glomerular filtration and |
| 501, MK0767, KRP-297, AZD6610 | carcinogenic effects in mice) |

Other drugs developed based on PPARs are shown in Table 3.

TABLE 3
Drugs and Combinations developed based on PPARs

| | |
|---|---|
| DRUG NAME | GENERIC NAME |

MARKETED

| | |
|---|---|
| (atorvastatin + ezetimibe + | (atorvastatin [INN] + ezetimibe + |
| fenofibrate) | fenofibrate) |
| (fenofibrate + pravastatin sodium) | (fenofibrate + pravastatin sodium) |
| (fenofibrate + rosuvastatin | (fenofibrate + rosuvastatin |
| calcium) | calcium) |
| (fenofibrate + rosuvastatin) | (fenofibrate + rosuvastatin [INN]) |
| (fenofibrate + simvastatin) | (fenofibrate + simvastatin) |
| (fenofibrate + pitavastatin) | (fenofibrate + pitavastatin) |
| (gliclazide + metformin | (gliclazide [INN] + metformin |
| hydrochloride + pioglitazone | hydrochloride + pioglitazone |
| hydrochloride) | hydrochloride) |
| (gliclazide + metformin | (gliclazide [INN] + metformin |
| hydrochloride + rosiglitazone) | hydrochloride + rosiglitazone |
| | [INN]) |
| (gliclazide SR + metformin | (gliclazide [INN] + metformin |
| hydrochloride SR + pioglitazone | hydrochloride + pioglitazone |
| hydrochloride) | hydrochloride) |
| (gliclazide SR + metformin SR + | (gliclazide [INN] + metformin + |
| pioglitazone) | pioglitazone [INN]) |
| glimepiride + metformin SR + | glimepiride + metformin + |
| pioglitazone | pioglitazone [INN] |
| (metformin ER + pioglitazone) | (metformin + pioglitazone [INN]) |
| (metformin hydrochloride + | (metformin hydrochloride + |
| pioglitazone) | pioglitazone [INN]) |
| (metformin hydrochloride + | (metformin hydrochloride + |
| rosiglitazone maleate) | rosiglitazone maleate) |
| (metformin hydrochloride + | (metformin hydrochloride + |
| pioglitazone hydrochloride) | pioglitazone hydrochloride) |
| (fenofibrate + metformin | (fenofibrate + metformin |
| hydrochloride) | hydrochloride) |
| (gliclazide + rosiglitazone) | (gliclazide [INN] + rosiglitazone |
| | [INN]) |
| (glimepiride + pioglitazone) | (glimepiride + pioglitazone [INN]) |
| (alogliptin benzoate + pioglitazone | (alogliptin benzoate + pioglitazone |
| hydrochloride) | hydrochloride) |
| ciprofibrate | ciprofibrate |
| fenofibrate | fenofibrate |
| gemfibrozil | gemfibrozil |
| bezafibrate SR | bezafibrate |
| clinofibrate | clinofibrate [INN] |
| clofibrate | clofibrate |
| clofibrate | clofibrate |
| choline fenofibrate | choline fenofibrate |
| saroglitazar | saroglitazar [INN] |
| lobeglitazone | lobeglitazone [INN] |
| zaltoprofen | zaltoprofen [INN] |
| pemafibrate | pemafibrate [INN] |

CLINICAL STAGE

| | |
|---|---|
| pemafibrate + tofogliflozin | pemafibrate [INN] + tofogliflozin |
| MA-0211 | |
| REN-001 | |
| EHP-101 | |
| ZYH-7 | |
| elafibranor | elafibranor |
| NC-2400 | |
| MA-0217 | |
| T-3D959 | |
| CHS-131 | |
| efatutazone | efatutazone [INN] |
| OMS-405 | |
| seladelpar lysine | seladelpar lysine |
| leriglitazone hydrochloride | leriglitazone hydrochloride |
| CS-038 | |

PRECLINICAL OR INACTIVE

| | |
|---|---|
| AU-9 | |
| BIO-201 | |
| BIO-203 | |
| BR-101549 | |
| CDIM-9 | |
| CNB-001 | |
| ELB-00824 | |
| ETI-059 | |
| KR-62980 | |
| MA-0204 | |
| PLX-300 | |
| RB-394 | |
| SR-10171 | |
| sulindac | sulindac |
| ZG-0588 | |
| A-91 | |
| AIC-47 | |
| CDE-001 | |
| CDIM-1 | |
| CDIM-5 | |
| CDIM-7 | |
| OP-601 | |
| (azilsartan + pioglitazone | (azilsartan + pioglitazone |
| hydrochloride) | hydrochloride) |
| ADC-3277 | |
| ADC-8316 | |
| ARH-049020 | |
| arhalofenate | arhalofenate |
| ATX-08001 | |
| AVE-0897 | |
| AZD-6610 | |
| BP-1107 | |
| CG-301269 | |
| CLC-3000 | |
| CLC-3001 | |
| CNX-013B2 | |
| CP-778875 | |
| CS-1050 | |
| CXR-1002 | |
| DB-900 | |
| DJ-5 | |
| DRF-10945 | |

DRUGS TO AGONIZE PPAR FOR TYPE 2 DIABETES

| | |
|---|---|
| etalocib | etalocib |
| farglitazar | farglitazar |
| GED-0507 | |
| indeglitazar | indeglitazar |
| K-111 | |
| KD-3010 | |
| KD-3020 | |
| KRP-101 | |
| LY-518674 | |
| LY-518674 | |
| mesalamine | mesalamine |
| NIP-222 | |
| NP-774 | |
| NS-220 | |
| PAM-1616 | |
| PBI-4547 | |
| PBI-4547 | |
| PBI-4547 | |
| peroxibrate | |
| PN-2034 | |
| PPM-201 | |
| PPM-202 | |
| romazarit | romazarit |

### Metformin

Metformin is an oral biguanide drug used to treat type 2 diabetes and polycystic ovary syndrome. The drug has multiple functions though the actual mechanism of action is not clearly defined. It appears to work by reducing insulin resistance in the liver and muscle tissue, suppressing glucose production, and increasing glucose uptake into tissue. Metformin treatment is associated with weight loss and metformin can be used to offset weight gain caused by the antipsychotic medications. In addition to its lowering of blood glucose, metformin has been found to have anti-inflammatory and antioxidant properties.

Metformin has been shown to act as both an anti-depressant and as a neuroprotective compound. In women with PCOS, metformin has been found to reduce the risk of major depression. Additional studies have also shown the potential for metformin as a sole or adjunct therapy in depression. The anti-depressant functions of metformin have been hypothesized to be due to owing to its anti-inflammatory, antioxidant, and neuroprotective activity. Alternatively, metformin's proposed mechanism of action is to reduce levels of BCAAs that would inhibit the entry of the amino acid tryptophan into the brain.

Metformin is under investigation for neurodegenerative diseases including AD and PD. Individuals with type 2 diabetes treated with metformin have a lower risk of developing dementia. Short term pilot studies have indicated that metformin may improve cognitive function. However, not all studies have found metformin to be beneficial in neurodegenerative diseases. It is possible that metformin treatment is beneficial in select subgroups of subjects.

### Triheptanoin

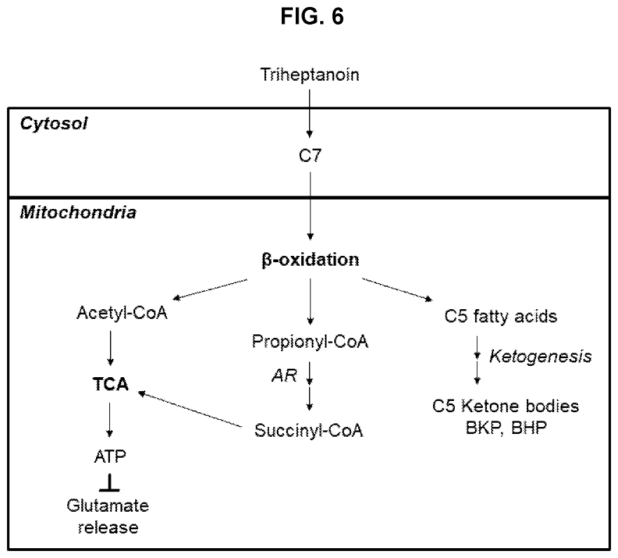

In the past 15 years the potential of triheptanoin for the treatment of several human diseases in the area of clinical nutrition has grown considerably. It is sold under the brand name DOJOLVI®. Use of this triglyceride of the odd-chain fatty acid heptanoate has been proposed and applied for the treatment of several conditions in which the energy supply from citric acid cycle intermediates or fatty acid degradation are impaired. Neurological diseases due to disturbed glucose metabolism or metabolic diseases associated with impaired β-oxidation of long chain fatty acid may especially take advantage of alternative substrate sources offered by the secondary metabolites of triheptanoin. Epilepsy due to deficiency of the GLUT1 transporter, as well as diseases associated with dysregulation of neuronal signalling, have been treated with triheptanoin supplementation, and very recently the advantages of this oil in long-chain fatty acid oxidation disorders have been reported. Triheptanoin is also used clinically in humans to treat inherited metabolic diseases, such as pyruvate carboxylase deficiency and carnitine palmitoyltransferase II deficiency. Triheptanoin is also used for the treatment of children and adults with confirmed long-chain fatty acid oxidation disorders (LC-FAOD) (see FIG. 6).

Triheptanoin is a triglyceride of three odd-chain fatty acid (heptanoate, C₇) is synthesized from castor bean oil originally used in the human food industry as a tasteless additive to dairy products or as an emollient in cosmetics. After cleavage by intestinal lipases heptanoate is absorbed by the gastrointestinal tract and metabolized predominantly in the liver. Because of its fast metabolism and anaplerotic potential triheptanoin use has been proposed for several diseases where enhanced energy production improves the clinical course of the disease. In recent years, the number of diseases treated with triheptanoin has steadily increased.

When triheptanoin (C₇ fatty acid) oil is ingested, it is hydrolyzed to 1 glycerol molecule and 3 molecules of heptanoate (C₇) that are metabolized by the liver. The catabolism of heptanoate produces acetyl-CoA and propionyl-CoA, which fuel the Krebs cycle. Heptanoate is more effective in fueling the Krebs cycle than even-chain fatty acids such as octanoate, which are metabolized to acetyl-CoA only. The effectiveness of heptanoate over even-chain fatty acids is therefore attributable to the importance of propionyl-CoA in serving as a substrate for gluconeogenesis in the liver and kidneys and as an anaplerotic substrate to fill the Krebs cycle in all tissues. In addition, oxidation of heptanoate in the liver leads to the export of C5 ketone bodies, which can be metabolized in peripheral tissues or in the brain to produce acetyl-CoA and propionyl-CoA. Likewise, the anaplerotic property of Triheptanoin has been used in several preclinical and clinical trials. Besides its effect on peripheral energy metabolism, especially fatty acid b-oxidation, triheptanoin has been shown to exert anticonvulsant effects in mouse models of epilepsy and affect neurotransmitter concentrations in subjects with pyruvate carboxylase deficiency.

Since triheptanoin is composed of odd-carbon fatty acids, it can produce ketone bodies with five carbon atoms, as opposed to even-carbon fatty acids which are metabolized to ketone bodies with four carbon atoms. The five-carbon ketones produced from Triheptanoin are beta-ketopentanoate and beta-hydroxypentanoate. Each of these ketone bodies easily crosses the blood-brain barrier and enters the brain.

Similar to the treatment with C₇-containing triglyceride, C₉-containing oil could also be used for treatment of neuropsychiatric diseases. MCFA are contributed by MCT (coconut or palm kernel) or fatty acids such as caprylic acid, while long chain fatty acids are contributed by vegetable oils (canola, safflower, peanuts, soybean, cotton seed, corn oil, etc.).

### Ketone Bodies

Ketone bodies are endogenously produced in the liver and serve as energy substrates for the brain under normal physiological conditions. Ketone bodies are water-soluble molecules that contain the ketone groups produced from fatty acids by the liver (ketogenesis). They are readily transported into tissues outside the liver, where they are converted into acetyl-CoA (acetyl-Coenzyme A), which then enters the TCA cycle and is oxidized for energy. Ketone bodies in the brain are used to convert acetyl-CoA into long-chain fatty acids. These liver-derived ketone groups include acetoacetic acid (acetoacetate), beta-hydroxybutyrate, and acetone, a spontaneous breakdown product of acetoacetate. However, under acylcarnitine homeostasis disruption and mitochondrial energy defects, ketone bodies (e.g., β-hydroxybutyrate (BHB)) can be supplemented for treatment of neuropsychiatric diseases. For example, in depression, higher levels of 2-hydroxybutyric acid and 3-hydroxybutyric acid will correlate with improved anxiety and depression severity.

### Short Chain and Medium Chain Fatty Acids

Short- and medium-chain fatty acids (SCFAs and MCFAs), independently of their cellular signaling functions, are important substrates of the energy metabolism and anabolic processes in mammals. SCFAs are mostly generated by colonic bacteria and are predominantly metabolized by enterocytes and liver, whereas MCFAs arise mostly from dietary triglycerides, among them milk and dairy products. A common feature of SCFAs and MCFAs is their carnitine-independent uptake and intramitochondrial activation to acyl-CoA thioesters. Contrary to long-chain fatty acids, the cellular metabolism of SCFAs and MCFAs depends to a lesser extent on fatty acid binding proteins. SCFAs and MCFAs modulate tissue metabolism of carbohydrates and lipids, as manifested by a mostly inhibitory effect on glycolysis and stimulation of lipogenesis or gluconeogenesis. SCFAs and MCFAs exert no or only weak protonophoric and lytic activities in mitochondria and do not significantly impair the electron transport in the respiratory chain. SCFAs and MCFAs modulate mitochondrial energy production by two mechanisms: they provide reducing equivalents to the respiratory chain and partly decrease efficacy of oxidative ATP synthesis.

Along with their role as energy-supplying fuel, SCFAs and MCFAs exhibit various regulatory and signaling functions. Butyrate and other SCFAs are known to induce apoptosis under specific conditions and thus to control cell proliferation. Currently, increasing attention is given to SCFAs with respect to their putative role in the pathogenesis of allergies, as well as autoimmune, metabolic, and neurological diseases. In the last two decades, the role of MCFAs as agonists of peroxisome proliferator activated receptors has also been characterized. Moreover, accumulating evidence indicates that SCFAs generated by the gut microbiota exert influence on food intake, thereby regulating energy homeostasis and body weight. SCFAs and MCFAs also play an important role in intracellular signaling and contribute to the regulation of cell metabolism. Finally, MCFAs and SCFAs can control cell death and survival.

### Short Chain Fatty Acids

Short chain fatty acids (SCFAs, mainly C2 to C6 fatty acids) are largely produced in the gut through bacterial fermentation of dietary fiber. These SCFAs could be freely transported to any cells or to the brain through one of the monocarboxylic acid transporters (MCT) to serve as energy substrates. When they are transported into mitochondria, a certain type of ligase converts them into acyl CoA to serve as TCA cycle substrate (e.g., acetyl CoA) or be used for synthesis of succinyl CoA (e.g., propionyl CoA). Under some conditions, these SCFAs can be supplemented using mixed chain triacylglycerols to the subjects as energy substrates.

TABLE 4
Short Chain Fatty Acids

| | | | Salt/Ester | Salt/Ester | | | Mol. | |
|---|---|---|---|---|---|---|---|---|
| Lipid | Common | Systematic | Common | Systematic | Mol. | Struct. | Wt. | |
| Number | Name | Name | Name | Name | Form. | Form. | (g/mol) | Structure |
| C1:0 | Formic acid | Methanocic acid | Formate | Methanoate | CH₂O₂ | HCOOH | 46.1 | 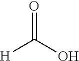 |
| C2:0 | Acetic acid | Ethanoic acid | Acetate | Ethanoate | C₂H₄O₂ | CH₃COOH | 60.1 | 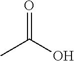 |
| C3:0 | Propionic acid | Propanoic acid | Propanoate | Propanoate | C₃H₆O₂ | CH₃CH₂COOH | 74.1 | 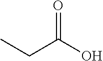 |
| C4:0 | Butyric acid | Butanoic acid | Butyrate | Butanoate | C₄H₈O₂ | CH₃(CH₂)₂COOH | 88.1 | 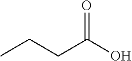 |
| C4:0 | Isobutyric acid | 2-methyl propanoic acid | Isobutyrate | 2-methyl propanoate | C₄H₈O₂ | (CH₃)₂CHCH₂COOH | 88.1 | 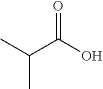 |
| C5:0 | Valeric acid | Pentanoic acid | Valerate | Pentanoate | C₅H₁₀O₂ | CH₃(CH₂)₃COOH | 102.1 | 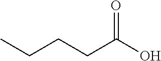 |
| C5:0 | Isovaleric Acid | 3-methyl butanoic acid | Isovalerate | 3-methyl butanoate | _{C5}H₁₀O₂ | (CH₃)₂CHCH₂COOH | 102.1 | 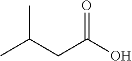 |

Medium Chain Fatty Acid: Even (C₆-C₁₂) and Odd Chain Fatty Acids (C₇, C₉)

The ketones, β-hydroxybutyrate (β-HB) and acetoacetate (AcAc), are produced by the liver during fasting or dietary carbohydrate restriction. During long-term fasting, ketones can provide up to 80% of the brain's energy requirements. Aside from energy or carbohydrate restriction, one common way to increase plasma ketones is by ingesting medium chain triglycerides (MCT) that provide medium chain fatty acids (MCFA), i.e., saturated fatty acids of 6-12 carbons in chain length. Several different MCFA are present in mammalian milk and in coconut oil or palm oil. MCFA are ketogenic because they are more rapidly metabolized than long chain fatty acids. In contrast to long chain fatty acids (14 carbons), which are absorbed via the lymphatic system and incorporated into circulating chylomicrons before reaching the liver, 8 and 10 carbon MCFA are thought to reach the liver directly via the portal vein. Eight carbon MCFA also cross the mitochondrial inner membrane without carnitine-dependent transport, allowing them to be more rapidly β-oxidized compared to long chain fatty acids.

### Branched Chain Amino Acids

Branched chain amino acids (BCAAs) are very important nutrients which serve as the substrates for biosynthesis of TCA cycle intermediates (e.g., succinyl CoA). It has been well documented that BCAAs are deficient in subjects with neuropsychiatric diseases. Therefore, supplementation of BCAAs and analogs, including leucine, isoleucine, and valine and non-proteinogenic BCAAs such as 2-aminoisobutyric acid could be used for treatment of neuropsychiatric diseases in combination with other drugs.

The proteinogenic branched-chain amino acids (BCAAs) leucine, isoleucine and valine are the most hydrophobic of the amino acids and belong to the nine essential amino acids. BCAA metabolism is directly connected to energy metabolism and oxidative BCAAs degradation leads to Krebs cycle intermediates. Furthermore, BCAAs have anabolic effects on protein metabolism through increasing the rate of protein synthesis and decreasing the rate of protein degradation in resting human muscles. Thus, even during recovery from endurance sports, branched-chain amino acids have anabolic effects in human muscles.

Recent study results underline the widely recognized significance of BCAAs as specific biomarkers of health and disease. Thus, previous studies suggest that BCAAs are associated with the risk of cardiovascular disease, end-stage renal failure, and ischemic stroke. In addition, circulating levels of BCAAs may have the potential to predict populations at risk for cardiometabolic disease and mortality from ischemic heart disease. In subjects suffering from cardiovascular diseases BCAAs are associated with mortality after cardiac catheterization. Circulating concentrations of valine and leucine are decreased in end-stage renal failure. In regard to cerebrovascular diseases, it needs to be mentioned that plasma BCAA levels are significantly decreased in subjects with transient ischemic attack or acute ischemic stroke, and low BCAA concentrations deteriorate outcomes in ischemic stroke subjects. In addition, BCAA administration has a beneficial effect on hepatic encephalopathy. It has been discovered that low levels of BCAA, along with low levels of short chain acetyl carnitines derived from BCAA, are found in AD subjects and in depressed subjects, and that an increase in their levels correlates with better outcomes.

### NAD

NAD⁺/NADH is a compound involved in many metabolic pathways that are related to neuroprotection and, in turn, its intake could be considered a promising approach to treatment of neurodegenerative diseases. Indeed, acting in glycolysis, Krebs cycle, and oxidative phosphorylation, it can contribute to preserve cognitive functions, representing a promising compound for AD treatment. Furthermore, the role of Nicotinamide Riboside, a NAD+ precursor, has been investigated in vivo in AD models and in vitro in PD and ALS mutated cells, providing a wide range of positive outcomes, from mitochondrial biogenesis enhancement to ROS production inhibition, reflected in cognitive improvement due to an increase of synaptic plasticity for what concerns AD mice.

### Riboflavin and FAD

Riboflavin, also known as vitamin B2, is a vitamin found in food and used as a dietary supplement. Flavin adenine dinucleotide (FAD) is a redox-active coenzyme associated with various proteins, which is involved with several enzymatic reactions in metabolism. A flavoprotein is a protein that contains a flavin group, which may be in the form of FAD or flavin mononucleotide (FMN). Many flavoproteins are known: components of the succinate dehydrogenase complex, α-ketoglutarate dehydrogenase, and a component of the pyruvate dehydrogenase complex.

FAD can exist in four redox states, which are the flavin-N(5)-oxide, quinone, semiquinone, and hydroquinone. FAD is converted between these states by accepting or donating electrons. FAD, in its fully oxidized form, or quinone form, accepts two electrons and two protons to become FADH₂ (hydroquinone form). The semiquinone (FADH) can be formed by either reduction of FAD or oxidation of FADH₂ by accepting or donating one electron and one proton, respectively. Some proteins, however, generate and maintain a superoxidized form of the flavin cofactor, the flavin-N(5)-oxide.

### Combinations

In other embodiments, the therapeutic intervention comprises the use of a combination of compounds within this class of therapies and or in combination with therapies currently used for treatment of CNS disease such as SSRIs or ketamine for treatment of depression and new classes of therapies being developed for treatment resistant depression. In some embodiments, the combination of selected compounds is based on metabolic profile defects noted in each subgroup of subjects (e.g., metabotype and genotype to inform about treatment selection).

In other embodiments, the therapy comprises a drug or drug combination. Examples of such drug or drug combinations include, but are not limited to, carnitine/L-acetyl carnitine and all structural analogs to facilitate transport and uptake, short chain acylcarnitines C₂-C₅, PPARα agonists, PPARγ agonists, PPARδ agonists, PPAR dual agonists, PPAR pan agonists, metformin, triheptanoin, ketone bodies, short chain fatty acids, medium chain fatty acids, even (C₆-C₁₂), medium chain fatty acids, odd chain fatty acids (C₇, C₉), branched chain amino acids, cofactors, NAD, riboflavin, FAD, Q10, carnitine, or combinations thereof. Of special interest are combinations between cannabinoid and cannabinoid-like compounds PEA, acylethanolamide natural compounds that also target cannabinoid receptors including linolenic acid (LEA), AA (AEA), docosatetraenoic acid (DEA), DHA (DHEA), and oleic acid (OEA); carnitine/L-acetyl carnitine, and combinations thereof. Other examples of suitable drugs and/or drug combinations are shown in Table 2.

One embodiment described herein is a method for stratifying and treating metabolic changes related to mitochondrial dysfunction, the method comprising: (a) obtaining a sample from one or more subjects suffering from a CNS disease or disorder; (b) analyzing the concentrations of mitochondrial metabolite biomarkers; (c) identifying mitochondrial metabolite biomarkers with abnormal concentration levels or abnormal concentration ratios compared to those of normal subjects; (d) identifying, analyzing, and cataloging enzymes or genes that are implicated in the metabolic, anabolic, catabolic, or transport pathways of the mitochondrial metabolite biomarkers with abnormal concentrations; and (e) administering diet changes, drugs, or combinations thereof to modulate the mitochondrial metabolite biomarkers with abnormal concentrations. In one aspect, the identifying, analyzing, and cataloging enzymes and genes that are implicated in the metabolic, anabolic, catabolic, or transport pathways of the mitochondrial metabolite biomarkers with abnormal concentrations comprises (a) performing a genetic screen analysis of the samples using GWAS analysis, Mendelian randomization analyses, univariable Mendelian randomization analyses, and/or multivariable Mendelian randomization analyses; (b) measuring the concentration levels and calculating the ratios of one or more mitochondrial biomarker metabolites in the samples, wherein the one or more mitochondrial biomarker metabolites; (c) comparing the genetic screen analyses to the measured concentration levels and calculated ratios of the one or more mitochondrial biomarker metabolites in the sample; (d) identifying a genetic basis of mitochondrial metabolic profile characteristics or any metabolic profile defects (SNPs/genetic variants in key enzymes and transporters) based on the genetic screen analyses; (e) determining if there is a causative genetic association between the measured concentration levels and calculated ratios of the one or more mitochondrial biomarker metabolites and depression in the subjects; and (f) stratifying the subjects afflicted with depression into subgroups based on their metabolic profiles, biomarker metabolites and ratios of biomarker metabolites, genetic screen analyses, and identified genetic associations.

Another embodiment described herein is a method for identifying genetics changes related to mitochondrial dysfunction, the method comprising: (a) obtaining a sample from one or more subjects suffering from a CNS disease or disorder; (b) performing a genetic screen analysis of the samples using GWAS analysis, Mendelian randomization analyses, univariable Mendelian randomization analyses, and/or multivariable Mendelian randomization analyses; (c) measuring the concentration levels and calculating the ratios of one or more mitochondrial biomarker metabolites in the samples, wherein the one or more mitochondrial biomarker metabolites; (d) comparing the genetic screen analyses to the measured concentration levels and calculated ratios of the one or more mitochondrial biomarker metabolites in the sample; (e) identifying a genetic basis of mitochondrial metabolic profile characteristics or any metabolic profile defects (SNPs/genetic variants in key enzymes and transporters) based on the genetic screen analyses; (f) determining if there is a causative genetic association between the measured concentration levels and calculated ratios of the one or more mitochondrial biomarker metabolites and depression in the subjects; and (g) stratifying the subjects afflicted with depression into subgroups based on their metabolic profiles, biomarker metabolites and ratios of biomarker metabolites, genetic screen analyses, and identified genetic associations.

Another embodiment described herein is a genomics-based method for identifying genetic causative mechanisms in subjects suffering from depression, the method comprising: (a) obtaining a sample from the subjects; (b) performing a genetic screen analysis of the samples using GWAS analysis, Mendelian randomization analyses, univariable Mendelian randomization analyses, and/or multivariable Mendelian randomization analyses; (c) measuring the concentration levels and calculating the ratios of one or more mitochondrial biomarker metabolites in the samples, wherein the one or more mitochondrial biomarker metabolites are selected from carnitine, short-chain acylcarnitines, medium-chain acylcarnitines, or long-chain acylcarnitines; or combinations thereof; (d) comparing the genetic screen analyses to the measured concentration levels and calculated ratios of the one or more mitochondrial biomarker metabolites in the sample; (e) identifying a genetic basis of mitochondrial metabolic profile characteristics or any metabolic profile defects (SNPs/genetic variants in key enzymes and transporters) based on the genetic screen analyses; (f) determining if there is a causative genetic association between the measured concentration levels and calculated ratios of the one or more mitochondrial biomarker metabolites and depression in the subjects; and (g) stratifying the subjects afflicted with depression into subgroups based on their metabolic profiles, biomarker metabolites and ratios of biomarker metabolites, genetic screen analyses, and identified genetic associations. In one aspect, low concentration levels of the short-chain acylcarnitines (C2, C3) and high levels of medium-chain acylcarnitines (C8, C10) are identified to have a causative association in depression. In another aspect, high levels of medium-chain acylcarnitines (C8, C10) indicate inborn errors of metabolism. In another aspect, the affected genes/enzymes that generate a metabolic defect may include electron transfer flavoprotein dehydrogenase (ETFDH) and/or medium-chain acyl-CoA dehydrogenase (ACADM). In another aspect, the affected genes/genes that generate a metabolic defect include short-chain acyl-CoA dehydrogenase (ACADS) and/or long-chain acyl-CoA dehydrogenase (ACADL). In one aspect, the method further comprises administering to the stratified subjects an effective amount of a therapy to treat the depression, wherein the therapy is determined by the genetic basis of mitochondrial metabolic profile characteristics or metabolic profile defects and the association with measured levels of mitochondrial biomarker metabolites. In another aspect, the therapy comprises compounds that inhibit the enzymes ETFDH or ACADM.

Another embodiment described herein is a method for discriminating or distinguishing between mild and severe depression in a subject using any of the methods as described herein. In one aspect, the method comprises performing a genetic screen analysis of the subject using GWAS analysis, Mendelian randomization analyses, univariable Mendelian randomization analyses, and/or multivariable Mendelian randomization analyses. In another aspect, the method comprises measuring the concentration levels and calculating the ratios of one or more mitochondrial biomarker metabolites. In another aspect, the method comprises measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof. In another aspect, the method comprises identifying causative associations between the measured concentration levels and calculated ratios of the one or more mitochondrial biomarker metabolites and depression.

Another embodiment described herein is a method for genetically screening for defects in metabolic processes (e.g., acylcarnitine biosynthesis and transport; TCA cycle, glycolysis, fatty acid beta-oxidation, ketogenesis, urea cycle, or electron transport chain) associated with CNS diseases and disorders using any of the methods as described herein. In one aspect, the method comprises identifying SNPs and gene variants of key enzymes and transporters involved in mitochondrial metabolic processes. In another aspect, the defective metabolic process comprises acylcarnitine biosynthesis wherein low concentration levels of the short-chain acylcarnitines (C2, C3) and high levels of medium-chain acylcarnitines (C8, C10) are identified to have a causative association in depression. In another aspect, high levels of medium-chain acylcarnitines (C8, C10) indicate inborn errors of metabolism that might mimic in part common mechanisms with neuropsychiatric diseases. Other medium chain acylcarnitines C12 and enzymes implicated in their regulation are also implicated.

Another embodiment described herein is a method for screening for compounds that modulate the activity of mitochondrial enzymes or transporters (e.g., ETFDH; ACADS; ACADM; ACADL, and other enzyme involved in acylcarnitine regulation) using any of the methods as described herein. In one aspect, the method comprises querying a compound screen to a specific enzyme or transporter; identifying compound hits that interact with the enzyme or transporter; performing quantitative structure/activity relationship analyses; identifying important compound moieties; and optimizing lead compound hits.

Another embodiment described herein is a method for correcting metabolic defects to treat CNS diseases or disorders by administering medications, modulating diet, or providing lifestyle interventions using any of the methods as described herein.

Another embodiment described herein is a method for screening for acylcarnitine homeostasis defects and treating subjects with CNS diseases or disorders by measuring short-, medium-, and long-chain acylcarnitine metabolite levels; genetically screening for SNPs and other genetic variants of key metabolic enzymes and transporters; stratifying subjects based on their metabolic profile and genetic screening; and administering an effective amount of an appropriate therapy.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for the classification and treatment of a CNS disease in a subject, the method comprising one or more of the following:
   (a) identifying and stratifying subjects afflicted with a CNS disease into subgroups based on their metabolic profiles, biomarker metabolites and ratios of biomarker metabolites that define unique metabolic conditions related to change in mitochondrial function and common identity among subgroups of subjects;
   (b) evaluating the trajectory of disease within each stratified subgroup of subjects and their response to a therapeutic treatment;
   (c) identifying defects in transport and/or biosynthesis breakdown of biomarker metabolites within a metabolic pathway or across metabolic pathways using ratios of biomarker metabolites to inform about changes in enzyme activities or transporters; and
   (d) identifying genetic bases of metabolic profile characteristics or defects (SNPs/genetic variants in key enzymes and transporters) using mGWAS analysis.
Clause 2. The method of clause 1, further comprising one or more of the following:
   (a) using combined metabotype and genotype data to better stratify subjects with neuropsychiatric diseases and to inform about mechanisms and treatment selection;
   (b) suggesting a therapeutic approach to correct metabolic defects in metabolic profile in stratified subgroups of subjects; and
   (c) comparing and contrasting metabolic defects noted in inborn errors of metabolism that have neurological and CNS deficits and using knowledge gained in treatment of inborn errors of metabolism to inform treatment for CNS diseases.
Clause 3. The method of clause 1 or 2, further comprising administering to the stratified subgroups of subjects an effective amount of a therapy to prevent and/or treat the CNS disease affected by specified genetic metabolic defects.
Clause 4. A method for stratifying and treating a subject having a neurological disorder, or at risk of developing a neurological disorder, based on the subject's mitochondrial metabolic profile, the method comprising:
   obtaining a sample from the subject;
   measuring the concentration levels and calculating the ratios of one or more mitochondrial biomarker metabolites in the sample, wherein the one or more mitochondrial biomarker metabolites are selected from carnitine, short-chain acylcarnitines, medium-chain acylcarnitines, or long-chain acylcarnitines; ketone bodies; amino acids, branched chain amino acids; biogenic amines; glycerophospholipids; sphingolipids; short-chain fatty acids; endocannabinoids; eicosanoids; other metabolites of glycolysis, TCA cycle, fatty acid beta-oxidation, urea cycle, or ketogenesis; or combinations thereof;
   determining if the subject has a mitochondrial metabolic defect related to disrupted acylcarnitine homeostasis, TCA cycle, glycolysis, fatty acid beta-oxidation, ketogenesis, urea cycle, or combinations thereof based on the measured concentration levels and calculated ratios of the one or more mitochondrial biomarker metabolites in the sample as compared to a control sample; and stratifying the subject into a subgroup of subjects, wherein an individual subgroup of subjects is defined by a unique and specific mitochondrial metabolic profile based on the measured concentration levels and calculated ratios of the one or more mitochondrial biomarker metabolites in the sample as compared to a control sample and the mitochondrial metabolic defect determined for the subject.
Clause 5. The method of clause 4, further comprising administering to the subgroup of subjects an effective amount of a therapy to treat the neurological disease, wherein the therapy is determined by the unique and specific mitochondrial metabolic profile of the subgroup of subjects.
Clause 6. The method of clause 4, wherein the biomarker metabolite comprises one or more of: Carnitine; Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine); Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
   or combinations thereof.
Clause 7. The method of clause 6, wherein the mitochondrial metabolic defect is related to disrupted acylcarnitine homeostasis and comprises:
   lower concentration levels of all acylcarnitines and higher ratios of carnitine/C3:0, carnitine/C5:0, carnitine/C10:0, carnitine/C16:0, and carnitine/C18:1 acylcarnitines;
   lower ratios of short and medium chain vs. long chain acylcarnitines (including C3:0/C16:0, C5:0/C16:0, C10:0/C16:0, C3:0/C18:1, C5:0/C18:1, and C10:0/C18:1);
   lower ratios of short chain vs. medium and long chain acylcarnitines (including C3:0/C10:0, C5:0/C10:0, C3:0/C16:0, C5:0/C16:0, C3:0/C18:1 and C5:0/C18:1); or
   lower ratios of odd-numbered short chain acylcarnitines vs. even-numbered short chain acylcarnitines (e.g., C6) and medium and long chain acylcarnitines (including C3:0/C6:0, C5:0/C6:0, C3:0/C10:0, C5:0/C10:0, C3:0/C16:0, C5:0/C16:0, C3:0/C18:1 and C5:0/C18:1).
Clause 8. The method of clause 6, wherein the mitochondrial metabolic defect is related to disrupted acylcarnitine homeostasis and comprises:
   Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); and
   Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine).
Clause 9. The method of clause 5, wherein the therapy comprises one or more compounds selected from peroxisome proliferator-activated receptor (PPAR) agonists, PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acid: Even (C₆-C₁₂), medium chain fatty acid: Odd chain fatty acids (C7, C₉), branched chain amino acids, carnitine, acetyl carnitine, propionylcarnitine, short chain acylcarnitines (C₂₋₅), cofactors NAD Flavin FAD, and combinations thereof.
Clause 10. The method of clause 4, wherein the mitochondrial metabolic defect comprises a deficiency in amino acids (e.g., branched chain amino acids) and/or short chain fatty acids.
Clause 11. The method of clause 9, wherein the therapy comprises one or more compounds selected from branched chain amino acids, propionic acid, ketone bodies, short chain acylcarnitines (C₂₋₅), medium chain acylcarnitines, analogs thereof, and combinations thereof.
Clause 12. The method of clause 4, wherein the neurological disorder is a CNS disorder, depression, or treatment resistant depression.
Clause 13. A method for stratifying and treating a subject having a neurological or CNS disorder, or at risk of developing a CNS or neurological disorder, based on the subject's mitochondrial metabolic profile, the method comprising:
   obtaining a sample from the subject;
   measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof in the sample; and
   determining if the subject has a mitochondrial metabolic defect related to disrupted acylcarnitine homeostasis, TCA cycle, glycolysis, fatty acid beta-oxidation, ketogenesis, urea cycle, or combinations thereof based on the measured expression level and/or activity of mitochondrial enzymes and/or transporters in the sample as compared to a control sample.
Clause 14. The method of clause 13, further comprising stratifying the subject into a subgroup of subjects, wherein an individual subgroup of subjects is defined by a unique and specific mitochondrial metabolic profile based on the measured expression level and activity of mitochondrial enzymes and/or transporters in the sample as compared to a control sample and the mitochondrial metabolic defect determined for the subject.
Clause 15. The method of clause 14, further comprising administering to the subgroup of subjects an effective amount of a therapy to treat the neurological disease, wherein the therapy is determined by the unique and specific mitochondrial metabolic profile of the subgroup of subjects.
Clause 16. The method of clause 13, wherein the mitochondrial enzyme and/or transporter comprises gamma-butyrobetaine hydroxylase 1 (BBOX1), organic cation transporter novel family member 2 (OCTN2), very long chain acylCoA dehydrogenase (VLCAD), medium chain acylCoA dehydrogenase (MCAD), short chain acylCoA dehydrogenase (SCAD), carnitine palmitoyltransferase1/2 (CPT1/2), carnitine-acylcarnitine translocase (CACT), carnitine octanoyltransferase, acetyl-CoA carboxylase1/2 (ACC1/2), ATP citrate synthase (ACLY), peroxisome proliferator-activated receptor (PPARα/PPARγ), or combinations thereof.
Clause 17. The method of clause 16, wherein the mitochondrial metabolic defect is related to disrupted acylcarnitine homeostasis and comprises:
   CPT defects: lower concentration levels of all acylcarnitines and higher ratios of carnitine/C3:0, carnitine/C5:0, carnitine/C10:0, carnitine/C16:0, and carnitine/C18:1 acylcarnitines;
   VLCAD defects: lower ratios of short and medium chain vs. long chain acylcarnitines (including C3:0/C16:0, C5:0/C16:0, C10:0/C16:0, C3:0/C18:1, C5:0/C18:1, and C10:0/C18:1);
   MCAD defects: lower ratios of short chain vs. medium and long chain acylcarnitines (including C3:0/C10:0, C5:0/C10:0, C3:0/C16:0, C5:0/C16:0, C3:0/C18:1 and C5:0/C18:1); or SCAD defects: lower ratios of odd-numbered short chain acylcarnitines vs. even-numbered short chain acylcarnitines (e.g., C6) and medium and long chain acylcarnitines (including C3:0/C6:0, C5:0/C6:0, C3:0/C10:0, C5:0/C10:0, C3:0/C16:0, C5:0/C16:0, C3:0/C18:1 and C5:0/C18:1).
Clause 18. The method of clause 15, wherein the therapy comprises one or more compounds selected from peroxisome proliferator-activated receptor (PPAR) agonists, PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acid: Even (C₆-C₁₂), medium chain fatty acid: Odd chain fatty acids (C₇, C₉), branched chain amino acids, carnitine, acetyl carnitine, propionylcarnitine, short chain acylcarnitines (C₂₋₅), cofactors NAD Flavin FAD, and combinations thereof.
Clause 19. The method of clause 16, wherein the mitochondrial metabolic defect is related to disrupted acylcarnitine homeostasis and comprises disrupted BBOX1, OCTN2, and/or CPT1/2 expression and/or activity.
Clause 20. A method for treating a CNS or neuropsychiatric disease in a subject, the method comprising:
   obtaining a sample from the subject;
   determining the presence, concentration levels, and ratios of one or more biomarker metabolites related to mitochondrial function in the sample from the subject;
   comparing the presence, concentration levels, and ratios of one or more biomarker metabolites related to mitochondrial function in the sample from the subject to the presence, concentration levels, and ratios of the one or more biomarker metabolites in a control sample; and
   determining if the subject has a CNS or neuropsychiatric disorder, or has an increased risk of developing a CNS or neuropsychiatric disorder when the concentration levels and ratios of the one or more biomarker metabolites in the sample from the subject are different from (greater than or less than) the concentration levels and ratios of the one or more biomarker metabolites in a control sample.
Clause 21. The method of clause 20, further comprising:
   stratifying the subject into a subgroup of subjects based on the concentration levels and ratios of the one or more biomarker metabolites related to mitochondrial function in the sample, wherein each subgroup of subjects is defined by a unique and specific mitochondrial metabolic profile; and administering to the subgroup of subjects an effective amount of a therapy to treat the CNS or neuropsychiatric disease, wherein the therapy is determined by the unique and specific mitochondrial metabolic profile of each subgroup of subjects.
Clause 22. A method for targeting mitochondrial pathways related to oxidative stress, mitochondrial biogenesis, and mitochondrial membrane permeability and dynamics in a subject suffering from, or at risk of suffering from, one or more neurodegenerative diseases, the method comprising:
   obtaining a sample from the subject and determining the concentration levels and ratios of one or more mitochondrial biomarker metabolites in the sample from the subject;
   determining if the subject has a neurodegenerative disease, or has an increased risk of developing a neurodegenerative disease when the concentration levels and ratios of the one or more mitochondrial biomarker metabolites in the sample from the subject are different from (greater than or less than) the concentration levels and ratios of the one or more mitochondrial biomarker metabolites in a control sample;
   stratifying the subject into a subgroup of subjects based on the concentration levels and ratios of the one or more mitochondrial biomarker metabolites in the sample, wherein each subgroup of subjects is defined by a unique and specific mitochondrial metabolic profile; and
   administering to the subgroup of subjects an effective amount of a therapy to treat the neurodegenerative disease, wherein the therapy is determined by the unique and specific mitochondrial metabolic profile of the subgroup of subjects.
Clause 23. A method for preparing and analyzing a sample containing a biomarker metabolite useful for the analysis and identification of metabolic changes associated with a CNS or neuropsychiatric disease in a subject, the method comprising:
   obtaining a sample from a subject;
   performing metabolic analysis on the sample to detect the presence and concentration of one or more biomarker metabolites;
   comparing the presence and concentration levels of one or more biomarker metabolites in the sample from the subject to the concentration levels of the one or more biomarker metabolites in a control sample;
   determining whether the presence and concentration levels of one or more biomarker metabolites in the sample from the subject correlate with the incidence of a CNS or neuropsychiatric disease, or an increased risk of a CNS or neuropsychiatric disease.
Clause 24. A method for stratifying and treating a subject having a neurological disorder, or at risk of developing a neurological disorder, based on the subject's mitochondrial metabolic profile, the method comprising:
   obtaining a sample from the subject;
   measuring the concentration levels and calculating the ratios of one or more mitochondrial biomarker metabolites in the sample, wherein the one or more mitochondrial biomarker metabolites are selected from carnitine, short-chain acylcarnitines, medium-chain acylcarnitines, or long-chain acylcarnitines; ketone bodies; amino acids, branched chain amino acids; biogenic amines; glycerophospholipids; sphingolipids; short-chain fatty acids; endocannabinoids; eicosanoids; other metabolites of glycolysis, TCA cycle, fatty acid beta-oxidation, urea cycle, or ketogenesis; or combinations thereof;
   measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof in the sample; and
   determining if the subject has a mitochondrial metabolic related to disrupted acylcarnitine homeostasis, TCA cycle, glycolysis, fatty acid beta-oxidation, ketogenesis, urea cycle, or combinations thereof based on the measured concentration levels and calculated ratios of the one or more mitochondrial biomarker metabolites in the sample as compared to a control sample and/or genetic defect in one or more mitochondrial enzymes and/or transporters involved in acylcarnitine biosynthesis and transport, TCA cycle, glycolysis, fatty acid beta-oxidation, ketogenesis, urea cycle, or combinations thereof; and
   stratifying the subject into a subgroup of subjects, wherein an individual subgroup of subjects is defined by a unique and specific mitochondrial metabolic profile based on the measured concentration levels and calculated ratios of the one or more mitochondrial biomarker metabolites in the sample as compared to a control sample and the mitochondrial metabolic defect determined for the subject;
   administering to the subgroup of subjects an effective amount of a therapy to treat the neurological disease, wherein the therapy is determined by the unique and specific mitochondrial metabolic profile of the subgroup of subjects.
Clause 25. The method of clause 32, wherein the therapy comprises one or more compounds selected from branched chain amino acids, propionic acid, ketone bodies, short chain acylcarnitines (C₂₋₅), medium chain acylcarnitines, analogs thereof, and combinations thereof.
Clause 26. The method of clause 32, wherein the neurological disorder is a CNS disorder, depression, or treatment resistant depression.
Clause 27. The method of any one of clauses 1-26, wherein the therapy comprises one or more repurposed compounds that improve mitochondrial energetics to treat CNS or neuropsychiatric diseases.
Clause 28. The method of any one of clauses 1-27, wherein the therapy comprises one or more repurposed compounds that modulate mitochondrial energetics to treat neurodegenerative diseases.
Clause 29. The method of any one of clauses 1-28, wherein the therapy comprises one or more of HU-210; CP 55940; Win 55212-2; anandamide; 2-AG; Noladin ether; virodhamine; oleoylethanolamide; palmitoylethanolamide; PPARγ Agonists; PPARβ/δ Agonists; dual and pan PPAR agonists; chiglitazar (CS038); AVE0847; aleglitazar (R1439); 5-substituted 2-benzoylamino-benzoic acid derivatives (BVT-142); O-arylmandelic acid derivatives; azaindole-α-alkyloxyphenylpropionic acid; amide substituted/α-substituted β-phenylpropionic acid derivatives; 2-alkoxydihydrocinnamate derivative; α-aryloxy-α-methylhydrocinnamic acids (LYS1029); TZD18; α-aryloxyphenyl acetic acid derivatives; PLX249; muraglitazar; mesaglitazar; naveglitazar; ragaglitazar; farglitazar; imiglitazar; netoglitazone; compound 3q JTT-501; MK0767; KRP-297; AZD6610; (atorvastatin+ezetimibe+fenofibrate); (fenofibrate+pravastatin sodium); (fenofibrate+rosuvastatin calcium); (fenofibrate+rosuvastatin); (fenofibrate+simvastatin); (fenofibrate+pitavastatin); (gliclazide+metformin hydrochloride+pioglitazone hydrochloride); (gliclazide+metformin hydrochloride+rosiglitazone); (gliclazide SR+metformin hydrochloride SR+pioglitazone hydrochloride); (gliclazide SR+metformin SR+pioglitazone); glimepiride+metformin SR+pioglitazone; (metformin ER+pioglitazone); (metformin hydrochloride+pioglitazone); (metformin hydrochloride+rosiglitazone maleate); (metformin hydrochloride+pioglitazone hydrochloride); (fenofibrate+metformin hydrochloride); (gliclazide+rosiglitazone); (glimepiride+pioglitazone); (alogliptin benzoate+pioglitazone hydrochloride); ciprofibrate; fenofibrate; gemfibrozil; bezafibrate SR; clinofibrate; clofibrate; clofibrate; choline fenofibrate; saroglitazar; lobeglitazone; zaltoprofen; pemafibrate; pemafibrate+tofogliflozin; MA-0211; REN-001; EHP-101; ZYH-7; elafibranor; NC-2400; MA-0217; T-3D959; CHS-131; efatutazone; OMS-405; seladelpar lysine; leriglitazone hydrochloride; CS-038; AU-9; BIO-201; BIO-203; BR-101549; CDIM-9; CNB-001; ELB-00824; ETI-059; KR-62980; MA-0204; PLX-300; RB-394; SR-10171; sulindac; ZG-0588; A-91; AIC-47; CDE-001; CDIM-1; CDIM-5; CDIM-7; OP-601; (azilsartan+pioglitazone hydrochloride); ADC-3277; ADC-8316; ARH-049020; arhalofenate; ATX-08001; AVE-0897; AZD-6610; BP-1107; CG-301269; CLC-3000; CLC-3001; CNX-013B2; CP-778875; CS-1050; CXR-1002; DB-900; DJ-5; DRF-10945; etalocib; farglitazar; GED-0507; indeglitazar; K-111; KD-3010; KD-3020; KRP-101; LY-518674; LY-518674; mesalamine; NIP-222; NP-774; NS-220; PAM-1616; PBI-4547; PBI-4547; PBI-4547; peroxibrate; PN-2034; PPM-201; PPM-202; romazarit; metformin; triheptanoin; ketone bodies; short chain fatty acids; methanoic acid; ethanoic acid; propanoic acid; butanoic acid; 2-methyl propanoic acid; pentanoic acid; 3-methyl butanoic acid; medium chain fatty acids; medium even (C6-C12) chain fatty acids; medium odd (C7, C9) chain fatty acids; fatty acid ethanolamides; cannabinoids; branched chain amino acids; nicotinamide adenine dinucleotide (NAD+/NADH, NADP+/NADPH); riboflavin; flavin adenine dinucleotide (FAD); EC5026; GSK2256294; AR9281; TPPU; t-TUCB; Dronabinol; Epidiolex; Δ9-tetrahydrocannabinol; cannabidiol; Δ9-tetrahydrocannabinol+cannabidiol; SSR411298; PF-04457845; JNJ-42165279; URB597 (KDS-4103); ST4070 (Alfasigma); Nabilone; Um-PEA (FSD201); NEO6860; OEA (RiduZone); or combinations thereof.
Clause 30. The method of any one of clauses 1-29, wherein therapy comprises one or more of: Δ⁹-tetrahydrocannabinol (Δ⁹-THC; Dronabinol); Δ⁸-tetrahydrocannabinol (Δ⁸-THC); exo-tetrahydrocannabinol (Exo-THC); Δ⁹-tetrahydrocannabinol naphtoylester (Δ⁹-THC-NE); Δ⁸-tetrahydrocannabinol naphtoylester (Δ⁸-THC-NE); exo-tetrahydrocannabinol naphtoylester (Exo-THC-NE); Δ⁹-tetrahydrocannabinolic acid (THCA-A, THCA-B); Δ⁸-tetrahydrocannabinolic acid (Δ⁸-THCA-A, Δ⁸-THCA-B); (−)-cannabidiol ((−)-CBD)/(+)-cannabidiol ((+)-CBD); cannabidiol-2′,6′-dimethyl ether (CBDD); 4-monobromo cannabidiol (4-MBO-CBD); cannabidiolic acid (CBDA); cannabiquinone (CBQ); nabilone; cannabivarin (CBNV); cannabivarinic acid (CBNVA); cannabivarin naphtoylester (CBNV-NE); Δ⁹-tetrahydrocannabivarin (Δ⁹-THCBV); Δ⁸-tetrahydrocannabivarin (Δ⁸-THCBV); Δ⁹-tetrahydrocannabivarin naphtoylester (Δ⁹-THCV-NE); Δ⁸-tetrahydrocannabivarin naphtoylester (Δ⁸-THCV-NE); Δ⁹-tetrahydrocannabivarinic acid (Δ⁹-THCVA); Δ⁸-tetrahydrocannabivarinic acid (Δ⁸-THCVA); (−)-cannabidivarin ((−)-CBDV)/(+)-cannabidivarin ((+)-CBDV)); cannabidivarinic acid (CBDVA); cannabidivarin quinone (CBQV); cannabidibutol (CBDB); cannabidibutolic acid (CBDBA); cannabidibutol naphtoylester (CBDB-NE); Δ⁹-tetrahydrocannabidutol (Δ⁹-THCBDB); Δ⁸-tetrahydrocannabidutol (Δ⁸-THCBDB); Δ⁹-tetrahydrocannabidutolic acid (Δ⁹-THCBDBA); Δ⁸-tetrahydrocannabidutolic acid (Δ⁸-THCBDBA); Δ⁹-tetrahydrocannabidutol naphtoylester (Δ⁹-THCB-NE); Δ⁸-tetrahydrocannabidutol naphtoylester (Δ⁸-THCB-NE); cannabibutol (CBB); cannabibutolic acid (CBBA); Δ⁹-tetrahydrocannabibutol (Δ⁹-THCB); Δ⁸-tetrahydrocannabibutol (Δ⁸-THCB); Δ⁹-tetrahydrocannabibutoic acid (Δ⁹-THCBA); Δ⁸-tetrahydrocannabibutolic acid (Δ⁸-THCBA); Δ⁹-tetrahydrocannabibutol naphtoylester (Δ⁹-THCB-NE); Δ⁸-tetrahydrocannabibutol naphtoylester (Δ⁸-THCB-NE); cannabinol (CBN); cannabinolic acid (CBNA); 3-butylcannabinol (CBNB); 3-butylcannabinolic acid (CBNBA); cannabielsoin (CBE); cannabicitran (CBT); cannabicyclol (CBL); cannabicyclolic acid (CBLA); cannabicyclol butyl (CBLB); cannabicyclol butyric acid (CBLBA); cannabicyclolvarin (CBLV); cannabicyclolvarinic acid (CBLVA); cannabigerol (CBG); cannabigerolic acid (CBGA); cannabigerol butyl (CBGB); cannabigerol butyric acid (CBGBA); cannabichromene (CBC); cannabichromenic acid (CBCA); cannabichromene butyl (CBCB); cannabichromene butyric acid (CBCBA); cannabigerivarin (CBGV); cannabigerivarinic acid (CBGVA); cannabichromevarin (CBCV); cannabichromevarinic acid (CBCVA); other cannabinoids, or pharmaceutically acceptable salts, acids, esters, amides, hydrates, solvates, prodrugs, isomers, stereoisomers, tautomers, derivatives thereof, or combinations thereof.
Clause 31. The method of any one of clauses 1-30, wherein the therapy further comprises an anti-depressant selected from selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof.
Clause 32. The method of any one of clauses 1-31, wherein the therapy further comprises one or more of citalopram, escitalopram, duloxetine, fluoxetine, paroxetine, sertraline, trazodone, lorazepam, oxazepam, aripiprazole, clozapine, haloperidol, olanzapine, quetiapine, risperidone, ziprasidone, amitriptyline, amoxapine, desipramine, doxepin, imipramine, nortriptyline, protriptyline, trimipramine, or combinations thereof.
Clause 33. The method of any one of clauses 1-32, wherein the therapy further comprises ketamine or esketamine.
Clause 34. The method of any one of clauses 1-33, wherein the therapy comprises one or more compounds selected from cannabinoids, cannabinoid-like compounds, carnitine, acetyl carnitines, and combinations thereof.
Clause 35. A method for stratifying and treating metabolic changes related to mitochondrial dysfunction, the method comprising: (a) obtaining a sample from one or more subjects suffering from a CNS disease or disorder; (b) analyzing the concentrations of mitochondrial metabolite biomarkers; (c) identifying mitochondrial metabolite biomarkers with abnormal concentration levels or abnormal concentration ratios compared to those of normal subjects; (d) identifying, analyzing, and cataloging enzymes or genes that are implicated in the metabolic, anabolic, catabolic, or transport pathways of the mitochondrial metabolite biomarkers with abnormal concentrations; and (e) administering diet changes, drugs, or combinations thereof to modulate the mitochondrial metabolite biomarkers with abnormal concentrations. In one aspect, the identifying, analyzing, and cataloging enzymes and genes that are implicated in the metabolic, anabolic, catabolic, or transport pathways of the mitochondrial metabolite biomarkers with abnormal concentrations comprises (a) performing a genetic screen analysis of the samples using GWAS analysis, Mendelian randomization analyses, univariable Mendelian randomization analyses, and/or multivariable Mendelian randomization analyses; (b) measuring the concentration levels and calculating the ratios of one or more mitochondrial biomarker metabolites in the samples, wherein the one or more mitochondrial biomarker metabolites; (c) comparing the genetic screen analyses to the measured concentration levels and calculated ratios of the one or more mitochondrial biomarker metabolites in the sample; (d) identifying a genetic basis of mitochondrial metabolic profile characteristics or any metabolic profile defects (SNPs/genetic variants in key enzymes and transporters) based on the genetic screen analyses; (e) determining if there is a causative genetic association between the measured concentration levels and calculated ratios of the one or more mitochondrial biomarker metabolites and depression in the subjects; and (f) stratifying the subjects afflicted with depression into subgroups based on their metabolic profiles, biomarker metabolites and ratios of biomarker metabolites, genetic screen analyses, and identified genetic associations.
Clause 36. A method for identifying genetics changes related to mitochondrial dysfunction, the method comprising: (a) obtaining a sample from one or more subjects suffering from a CNS disease or disorder; (b) performing a genetic screen analysis of the samples using GWAS analysis, Mendelian randomization analyses, univariable Mendelian randomization analyses, and/or multivariable Mendelian randomization analyses; (c) measuring the concentration levels and calculating the ratios of one or more mitochondrial biomarker metabolites in the samples, wherein the one or more mitochondrial biomarker metabolites; (d) comparing the genetic screen analyses to the measured concentration levels and calculated ratios of the one or more mitochondrial biomarker metabolites in the sample; (e) identifying a genetic basis of mitochondrial metabolic profile characteristics or any metabolic profile defects (SNPs/genetic variants in key enzymes and transporters) based on the genetic screen analyses; (f) determining if there is a causative genetic association between the measured concentration levels and calculated ratios of the one or more mitochondrial biomarker metabolites and depression in the subjects; and (g) stratifying the subjects afflicted with depression into subgroups based on their metabolic profiles, biomarker metabolites and ratios of biomarker metabolites, genetic screen analyses, and identified genetic associations.
Clause 37. A genomics-based method for identifying genetic causative mechanisms in subjects suffering from depression, the method comprising: (a) obtaining a sample from the subjects; (b) performing a genetic screen analysis of the samples using GWAS analysis, Mendelian randomization analyses, univariable Mendelian randomization analyses, and/or multivariable Mendelian randomization analyses; (c) measuring the concentration levels and calculating the ratios of one or more mitochondrial biomarker metabolites in the samples, wherein the one or more mitochondrial biomarker metabolites are selected from carnitine, short-chain acylcarnitines, medium-chain acylcarnitines, or long-chain acylcarnitines; or combinations thereof; (d) comparing the genetic screen analyses to the measured concentration levels and calculated ratios of the one or more mitochondrial biomarker metabolites in the sample; (e) identifying a genetic basis of mitochondrial metabolic profile characteristics or any metabolic profile defects (SNPs/genetic variants in key enzymes and transporters) based on the genetic screen analyses; (f) determining if there is a causative genetic association between the measured concentration levels and calculated ratios of the one or more mitochondrial biomarker metabolites and depression in the subjects; and (g) stratifying the subjects afflicted with depression into subgroups based on their metabolic profiles, biomarker metabolites and ratios of biomarker metabolites, genetic screen analyses, and identified genetic associations. In one aspect, low concentration levels of the short-chain acylcarnitines (C2, C3) and high levels of medium-chain acylcarnitines (C8, C10) are identified to have a causative association in depression. In another aspect, high levels of medium-chain acylcarnitines (C8, C10) indicate inborn errors of metabolism. In another aspect, the affected genes/enzymes that generate a metabolic defect may include electron transfer flavoprotein dehydrogenase (ETFDH) and/or medium-chain acyl-CoA dehydrogenase (ACADM). In another aspect, the affected genes/genes that generate a metabolic defect include short-chain acyl-CoA dehydrogenase (ACADS) and/or long-chain acyl-CoA dehydrogenase (ACADL). In one aspect, the method further comprises administering to the stratified subjects an effective amount of a therapy to treat the depression, wherein the therapy is determined by the genetic basis of mitochondrial metabolic profile characteristics or metabolic profile defects and the association with measured levels of mitochondrial biomarker metabolites. In another aspect, the therapy comprises compounds that inhibit the enzymes ETFDH or ACADM.
Clause 38. A method for discriminating or distinguishing between mild and severe depression in a subject using any of the methods as described herein. In one aspect, the method comprises performing a genetic screen analysis of the subject using GWAS analysis, Mendelian randomization analyses, univariable Mendelian randomization analyses, and/or multivariable Mendelian randomization analyses. In another aspect, the method comprises measuring the concentration levels and calculating the ratios of one or more mitochondrial biomarker metabolites. In another aspect, the method comprises measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof. In another aspect, the method comprises identifying causative associations between the measured concentration levels and calculated ratios of the one or more mitochondrial biomarker metabolites and depression.
Clause 39. A method for genetically screening for defects in metabolic processes (e.g., acylcarnitine biosynthesis and transport; TCA cycle, glycolysis, fatty acid beta-oxidation, ketogenesis, urea cycle, or electron transport chain) associated with CNS diseases and disorders using any of the methods as described herein. In one aspect, the method comprises identifying SNPs and gene variants of key enzymes and transporters involved in mitochondrial metabolic processes. In another aspect, the defective metabolic process comprises acylcarnitine biosynthesis wherein low concentration levels of the short-chain acylcarnitines (C2, C3) and high levels of medium-chain acylcarnitines (C8, C10) are identified to have a causative association in depression. In another aspect, high levels of medium-chain acylcarnitines (C8, C10) indicate inborn errors of metabolism that might mimic in part common mechanisms with neuropsychiatric diseases. Other medium chain acylcarnitines C12 and enzymes implicated in their regulation are also implicated.
Clause 40. A method for screening for compounds that modulate the activity of mitochondrial enzymes or transporters (e.g., ETFDH; ACADS; ACADM; ACADL, and other enzyme involved in acylcarnitine regulation) using any of the methods as described herein. In one aspect, the method comprises querying a compound screen to a specific enzyme or transporter; identifying compound hits that interact with the enzyme or transporter; performing quantitative structure/activity relationship analyses; identifying important compound moieties; and optimizing lead compound hits.
Clause 41. A method for correcting metabolic defects to treat CNS diseases or disorders by administering medications, modulating diet, or providing lifestyle interventions using any of the methods as described herein.
Clause 42. A method for screening for acylcarnitine homeostasis defects and treating subjects with CNS diseases or disorders by measuring short-, medium-, and long-chain acylcarnitine metabolite levels; genetically screening for SNPs and other genetic variants of key metabolic enzymes and transporters; stratifying subjects based on their metabolic profile and genetic screening; and administering an effective amount of an appropriate therapy.

### EXAMPLES

### Example 1

### Role for Acylcarnitines in Depression and Treatment Outcomes

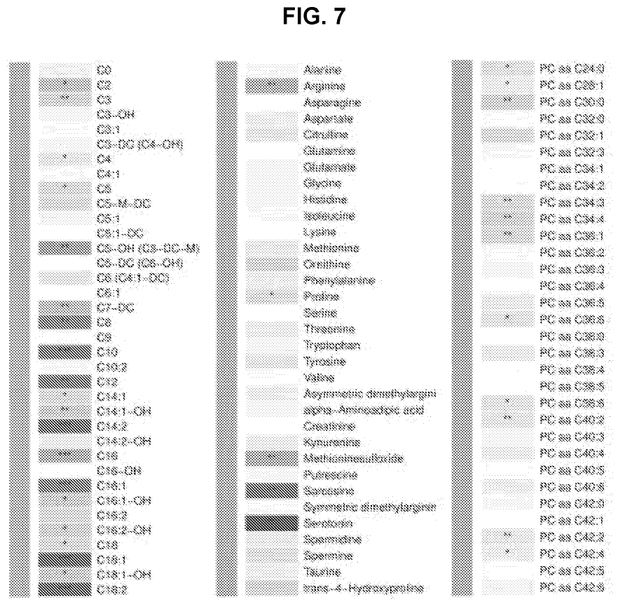

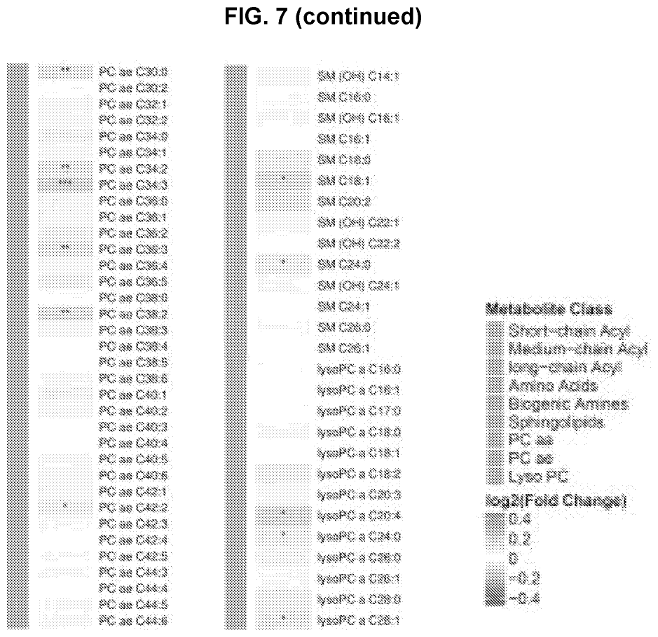

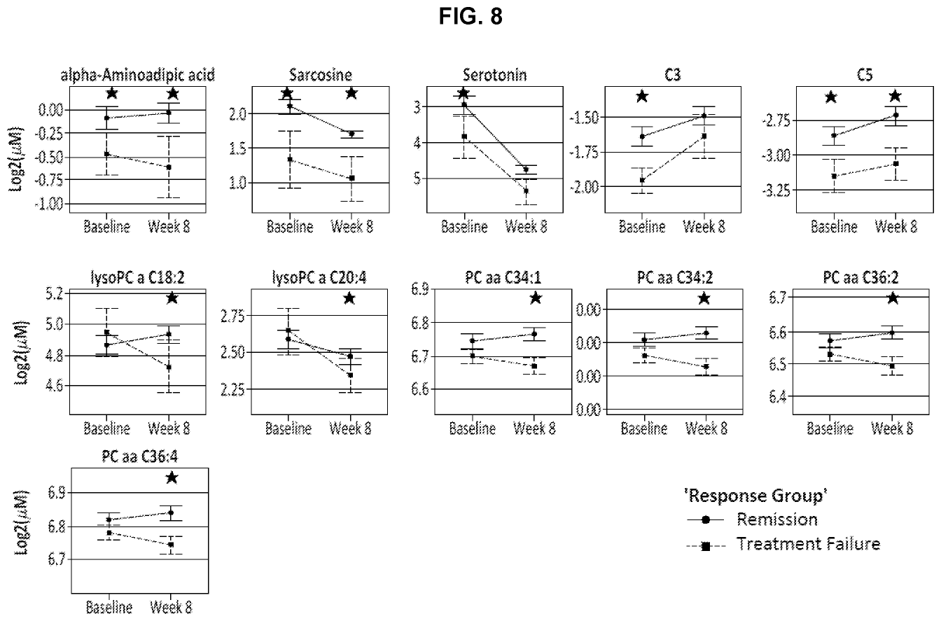

A targeted metabolomics approach was used utilizing a panel of 180 metabolites to gain insights into mechanisms of action and response to citalopram/escitalopram. Plasma samples from 136 participants with MDD enrolled into the Mayo Pharmacogenomics Research Network Antidepressant Medication Pharmacogenomic Study (PGRN-AMPS) were profiled at baseline and after 8 weeks of treatment. After treatment, increased levels of short-chain acylcarnitines and decreased levels of medium- and long chain acylcarnitines were observed, suggesting an SSRI effect on β-oxidation and mitochondrial function. Amines—including arginine, proline, and methionine-sulfoxide—were upregulated while serotonin and sarcosine were downregulated, suggesting an SSRI effect on urea cycle, one carbon metabolism and serotonin uptake. Eighteen lipids within the phosphatidylcholine (PC aa and ae) classes were upregulated. Changes in several lipid and amine levels correlated with changes in 17-item Hamilton Rating Scale for Depression scores (HRSD17). Differences in metabolic profiles at baseline and post-treatment were noted between participants who remitted (HRSD17≤7) and those who gained no meaningful benefits (<30% reduction in HRSD17). Remitters exhibited (a) higher baseline levels of C3, C5, alpha-aminoadipic acid, sarcosine and serotonin; and (b) higher week-8 levels of PC aa C34:1, PC aa C34:2, PC aa C36:2 and PC aa C36:4. These findings suggest that mitochondrial energetics—including acylcarnitine metabolism, transport and its link to β-oxidation—and lipid membrane remodeling may play roles in SSRI treatment response (see FIG. 7-8).

### Example 2

### Acylcarnitine Metabolomic Profiles Inform Clinically Defined Major Depressive Phenotypes

Acylcarnitines have important functions in mitochondrial energetics and β-oxidation and have been implicated to play a significant role in metabolic functions of the brain. This retrospective study examined whether plasma acylcarnitine profiles can help biochemically distinguish the three phenotypic subtypes of major depressive disorder (MDD): core depression (CD+), anxious depression (ANX+), and neurovegetative symptoms of melancholia (NVSM+).

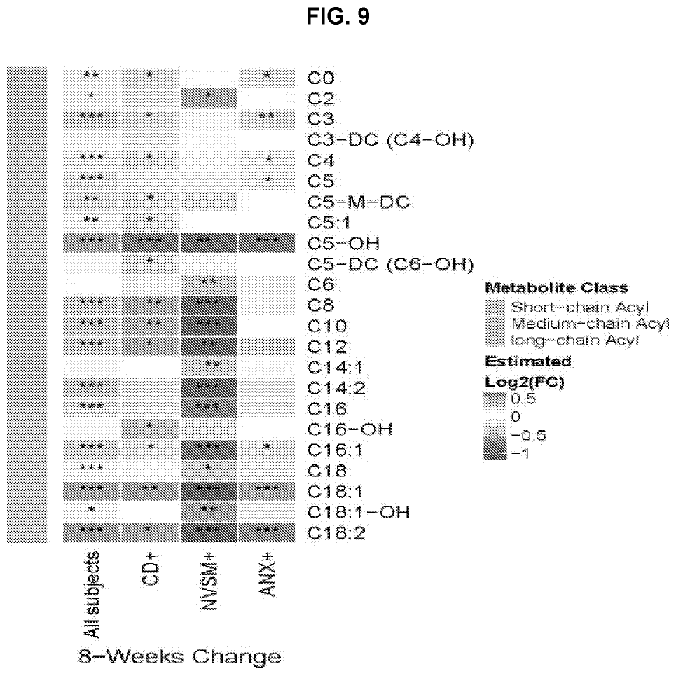

Depressed outpatients (n=240) from the Mayo Clinic Pharmacogenomics Research Network were treated with citalopram or escitalopram for eight weeks. Plasma samples collected at baseline and after eight weeks of treatment with citalopram or escitalopram were profiled for short-, medium- and long-chain acylcarnitine levels using AbsoluteIDQ®p180-Kit and LC-MS. Linear mixed effects models were used to examine whether acylcarnitine levels discriminated the clinical phenotypes at baseline or eight weeks post-treatment, and whether temporal changes in acylcarnitine profiles differed between groups (see FIG. 9).

Compared to ANX+, CD+ and NVSM+ had significantly lower concentrations of short- and long-chain acylcarnitines at both baseline and week 8. In NVSM+, the medium- and long-chain acylcarnitines were also significantly lower in NVSM+ compared to ANX+. Short-chain acylcarnitine levels increased significantly from baseline to week 8 in CD+ and ANX+, whereas medium- and long-chain acylcarnitines significantly decreased in CD+ and NVSM+.

In depressed subjects treated with SSRIs, β-oxidation, and mitochondrial energetics as evaluated by levels and changes in acylcarnitines may provide the biochemical basis of the clinical heterogeneity of MDD, especially when combined with clinical characteristics.

### Example 3

### Ketamine Induces Rapid Changes in Mitochondrial Pathways

Ketamine, at sub-anesthetic doses, is reported to rapidly decrease depression symptoms in subjects with treatment-resistant major depressive disorder (MDD). Many subjects do not respond to currently available antidepressants, (for example, serotonin reuptake inhibitors), making ketamine and its enantiomer, esketamine, potentially attractive options for treatment-resistant MDD. Although mechanisms by which ketamine/esketamine may produce antidepressant effects have been hypothesized on the basis of preclinical data, the neurobiological correlates of the rapid therapeutic response observed in subjects receiving treatment have not been established.

Here, a pharmacometabolomics approach was used to map global metabolic effects of these compounds in treatment-refractory MDD subjects upon 2 h from infusion with ketamine (n=33) or its S-enantiomer, esketamine (n=20). The effects of esketamine on metabolism were retested in the same subjects following a second exposure administered 4 days later. Two complementary metabolomics platforms were used to provide broad biochemical coverage. In addition, it was investigated as to whether changes in particular metabolites correlated with treatment outcome. Both drugs altered metabolites related to tryptophan metabolism (for example, indole-3-acetate and methionine) and/or the urea cycle (for example, citrulline, arginine and ornithine) at 2 h post infusion (q>0.25). In addition, changes in glutamate acylcarnitines and circulating phospholipids were observed that were significantly associated with decreases in depression severity. These data provide new insights into the mechanism underlying the rapid antidepressant effects of ketamine and esketamine and constitute some of the first detailed metabolomics mapping for these promising therapies.

### Example 4

### Genomics-Based Identification of a Potential Causal Role for Acylcarnitines Metabolism in Depression

### GWAS Summary Statistics and Instrument Selection

Summary statistics were obtained from large GWAS of international consortia. Summary statistics for ACs measured with a targeted metabolomics platform was retrieved from a GWAS in 9,363 samples from the Fenland study, a population-based cohort of adults recruited from general practice lists in Cambridgeshire, UK. The Psychiatric Genomics Consortium (PGC) performed an overarching meta-analysis of all available GWAS datasets with depression phenotypes (hereafter labeled as DEP) including established Major Depressive Disorder diagnosis or self-declared depression, totaling 246,363 cases and 344,901 controls.

AC summary statistics were processed by removing strand ambiguous SNPs and with MAF<1%. Variants overlapping with those reported in depression GWAS were clumped (10,000 kb window, r²=0.01, EUR population of 1000 Genomes used as linkage disequilibrium reference) to identify independent significantly (p<5.0×10⁻⁸) associated SNPs. For the present analyses, data were retained from 15 short-, medium-, and long-chain ACs with at least two independent associated SNPs.

### Genetic Architecture of Selected Acylcarnitines

Full GWAS summary statistics of ACs were used to derive two parameters related to their genetic architecture. SNP-heritability (h2SNP, the proportion of trait variance explained by the joint effect of all genotyped common SNPs) was estimated using linkage-disequilibrium score regression (LDSC). Pairwise genetic correlations (rg, determined by the number of SNPs and their level of concordance shared between two traits) between ACs were estimated using the high-definition likelihood (HDL) method recently developed as an extension of bivariate LDSC.

### Mendelian Randomization Analyses

Two-sample Mendelian randomization (2SMR) analyses based on GWAS summary statistics were performed to test the potential causal role of ACs on depression risk. For each acylcarnitine used as exposure, genome-wide significant independent SNPs were used as instruments. Selected SNPs were aligned on the positive strand for exposures and outcome (DEP). F-statistics (all F>10) indicated that the strength of selected genetic instruments was adequate. Lack of sample overlap across the two discovery GWAS reduced the likelihood of causal estimates biased toward the observational correlation.

First, a series of univariate 2SMR analyses were performed based on the inverse variance weighted (IVW) estimator, pooling SNP-exposure/SNP-outcome estimates inversely weighted by their standard error. Since IVW assumes that all SNPs are valid instruments or that the sum of the directional bias is zero, the robustness of significant results was tested in sensitivity analyses based on weighted median and MR-Egger estimators. The weighted median estimator is the median of the weighted empirical distribution function of individual SNP ratio estimates, providing a consistent effect estimates even if half of the instruments are invalid. The MR-Egger regression consists of a weighted linear regression similar to IVW relying on the InSIDE assumption (the magnitude of any pleiotropic effects should not correlate with the magnitude of the main effect), providing valid effect estimate even if all SNPs are invalid instruments under the ‘NOME’ assumption (uncertainty in the SNP-exposure association estimates is negligible). At least 10 genetic instruments are recommended to run adequately powered MR-Egger analyses. Furthermore, heterogeneity among included SNPs was tested via Cochran's Q test, single SNP, and leave-one-out SNP analyses. The presence of potential horizontal pleiotropy (a genetic instrument for exposure influencing the outcome by mechanisms other than exposure) was tested using the MR-Egger intercept and the MR-PRESSO (pleiotropy residual sum and outlier) method. The MR-PRESSO procedure includes several tests: a global test evaluating overall horizontal pleiotropy among all instruments; an outlier test evaluating the presence of specific outlier variants; a distortion test evaluating the significance of the distortion in causal estimates before/after removal of outlier variants. Moreover, in order to evaluate the impact of the strength of genetic instruments on previous results, univariate 2SMR analyses were repeated for a subset of seven ACs with instruments derived from larger discovery GWAS summary statics. Results from a previous mGWAS(27) (N=7,478) were meta-analyzed with those of the Fenland Study and the pooled summary statistics were used (supplemental methods) to derive genetic instruments for 7 of the 15 ACs examined in the present analyses. Finally, reversed univariable 2SMR analyses were performed to test the potential causal impact of depression liability on ACs circulating levels.

Due to high reciprocal genetic correlation, ACs significantly associated at nominal level with DEP risk in univariable analyses were carried forward in multivariable MR (MVMR), in which the association of each exposure instrument was conditioned on the others. Analyses were conducted in R v4.0.0 (R Project for Statistical Computing) using the MR-Base package. Statistical significance level was set at α=0.05, two-sided. False Discovery Rate (FDR) q-values according to the Benjamini-Hochberg procedure were additionally reported in univariable analyses testing separately all 15 ACs.

Selected data included GWAS summary statistics of 15 ACs (short-chain: C0, C2, C3, C4, C5; medium-chain: C6, C8, C9, C10, C10:1, C12; long-chain: C14:1, C16, C18:1, C18:2) with at least two independent associated SNPs (median N of associated SNPs=5).

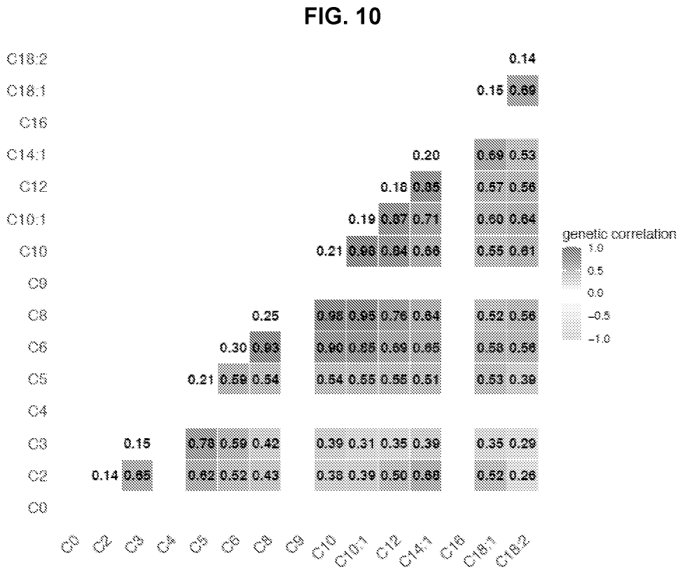

FIG. 10 shows genetic architecture parameters of the selected ACs. The diagonal reports h2SNP, which could be estimated as significantly different from 0 for eleven of the fifteen ACs examined. Estimates of h2SNP were substantially similar—although with large uncertainty likely due to the limited sample of the original GWAS—and varied from 0.14 for C2 (se=0.07) and C18:2 (se=0.05) to 0.30 (se=0.05) for C6. The heatmap displays pairwise rg between ACs. Genetic correlations were moderate (across chain lengths) to strong (within chain lengths) strong, in particular within the medium-chain ACs, with C8-C10 (rg=0.98, se=0.01) and C10-C10:1 (rg=0.98, se=0.17) correlations statistically not different from the unity.

### Univariable Mendelian Randomization Analyses

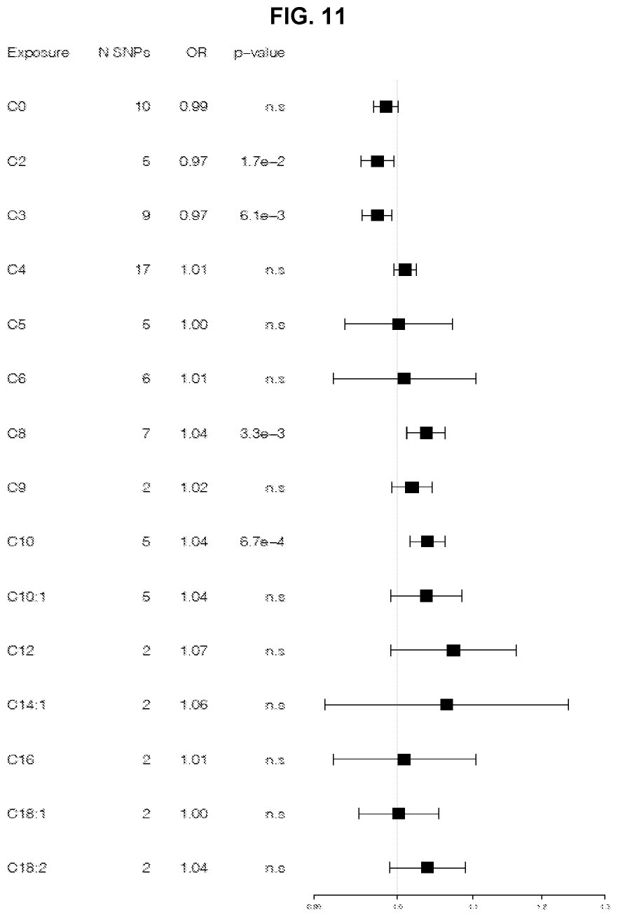

FIG. 11 shows the results of univariable 2SMR IVW analyses estimating the risk of DEP (expressed as odds ratios [ORs] and 95% confidence intervals [95% CIs]) per SD increase in genetically-predicted levels of (log)ACs. A lower risk of DEP was significantly associated with genetically-predicted higher levels of the short-chain ACs C2 (OR=0.97, 95% CIs=0.95-1.00, p=1.7×10⁻², q=0.06) and C3 (OR=0.97, 95% CIs=0.96-0.99, p=1.7×10⁻², q=0.03). Conversely, a higher DEP risk was associated with genetically-predicted higher levels of the medium-chain ACs C8 (OR=1.04, 95% CIs=1.01-1.06, p=3.3×10⁻³, q=0.02) and C10 (OR=1.04, 95% CIs=1.02-1.06, p=6.7×10⁻⁴, q=0.01).

Sensitivity analyses confirmed the robustness of these results: causal estimates obtained via weighted-median and MR-Egger estimators, tackling different patterns of MR assumption violation, were completely in line with those from IVW (Table 5). MR-Egger estimates were not statistically significant, likely due to the limited number of genetic instruments, which were lower than those recommended (N≥10) to achieve adequate statistical power. Nevertheless, MR-Egger causal estimates were highly convergent with those obtained by other 2SMR analyses. Furthermore, heterogeneity across SNPs was not statistically significant. Inspections of single-SNP and leave-one-out-SNP analyses plots for C8 reporting a Cochran's Q p-value=0.05, did not reveal the presence of outlier SNPs. MR-Egger intercept estimates and results from MR-PRESSO did not show statistically significant evidence of horizontal pleiotropy. Furthermore, univariable 2SMR analyses were repeated for 7 of the 15 ACs (short-chain: C3; medium-chain: C8, C9, C10:1; long-chain: C14:1, C18:1, C18:2) using GWAS summary statics derived from a meta-analyses of results from the Fenland Study and those from Draisma et al., Nat. Commun. 6: 1-9 (2015). The larger discovery GWAS (up to 16,832 samples) enabled the inclusion of relatively stronger individual instruments but not of a larger number of independently associated SNPs, further supporting the notion of a sparse genetic architecture for ACs characterized by few biologically relevant loci with relatively larger effect sizes. Analyses with the newly derived instruments for ACs confirmed statistically significant causal estimates for C3 (OR=0.96, 95% CIs=0.93-0.99, p=5.6×10⁻³) and C8 (OR=1.05, 95% CIs=1.02-1.08, p=2.7×10⁻³), with similar effect size as those obtained in the main analyses based on the Fenland Study summary statistics.

TABLE 5
Association between genetically-predicted levels of four acylcarnitines and risk
of depression obtained with different Mendelian Randomization estimators.

| | | | Inverse Variance | | | | | | | | | | | |
|---|---|---|---|---|---|---|---|---|---|---|---|---|---|---|
| | | | Weighted | | | | Weighted median | | | | MR-Egger | | | |
| Exposure | NSNPs | Outcome | OR | lCI | uCI | p-value | OR | lCI | uCI | p-value | OR | lCI | uCI | p-value |
| C2 | 5 | DEP | 0.97 | 0.95 | 1.00 | 1.7 × 10⁻² | 0.97 | 0.95 | 0.99 | 9.0 × 10⁻³ | 0.97 | 0.93 | 1.02 | 0.39 |
| C3 | 9 | DEP | 0.97 | 0.96 | 0.99 | 6.1 × 10⁻³ | 0.97 | 0.95 | 0.99 | 8.6 × 10⁻³ | 0.99 | 0.94 | 1.05 | 0.85 |
| C8 | 7 | DEP | 1.04 | 1.01 | 1.06 | 3.0 × 10⁻³ | 1.02 | 1.00 | 1.05 | 4.5 × 10⁻² | 1.02 | 0.97 | 1.08 | 0.39 |
| C10 | 5 | DEP | 1.04 | 1.02 | 1.06 | 6.7 × 10⁻⁴ | 1.03 | 1.01 | 1.06 | 1.3 × 10⁻² | 1.04 | 0.98 | 1.10 | 0.28 |

Odds ratios (ORs) and 95% confidence intervals (lCI, lower bound; uCI, upper bound) per SD increase in genetically-predicted levels of (log)ACs

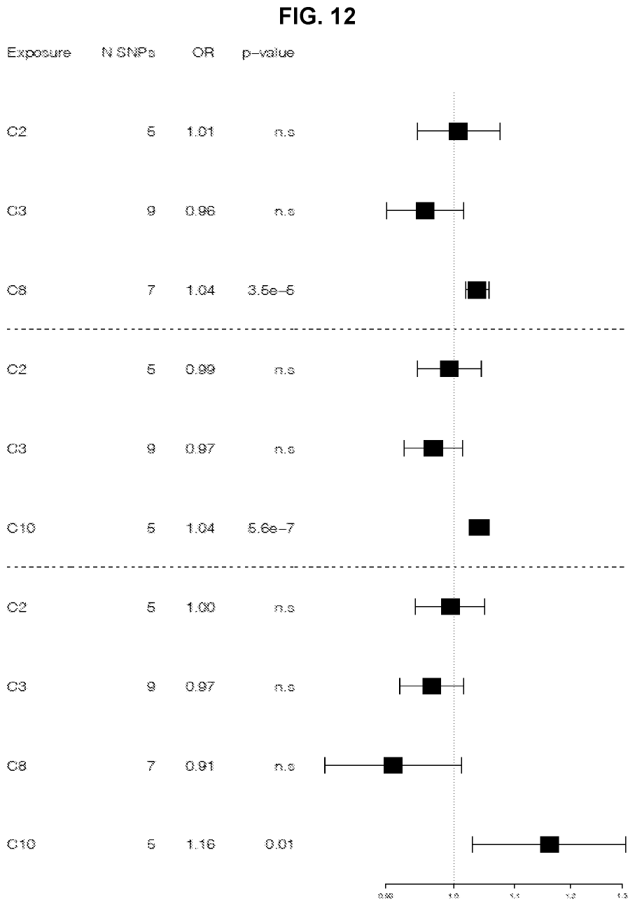

Finally, reversed 2SMR analyses were preformed to examine the potential causal role of DEP on AC levels. FIG. 12 depicts estimates representing change in SD of (log)ACs levels per doubling (2-fold increase) in the prevalence of the exposure. None of the causal estimates were statistically significant, indicating lack of evidence for a reversed causal effect of depression liability on ACs levels.

### Multivariable Mendelian Randomization Analyses

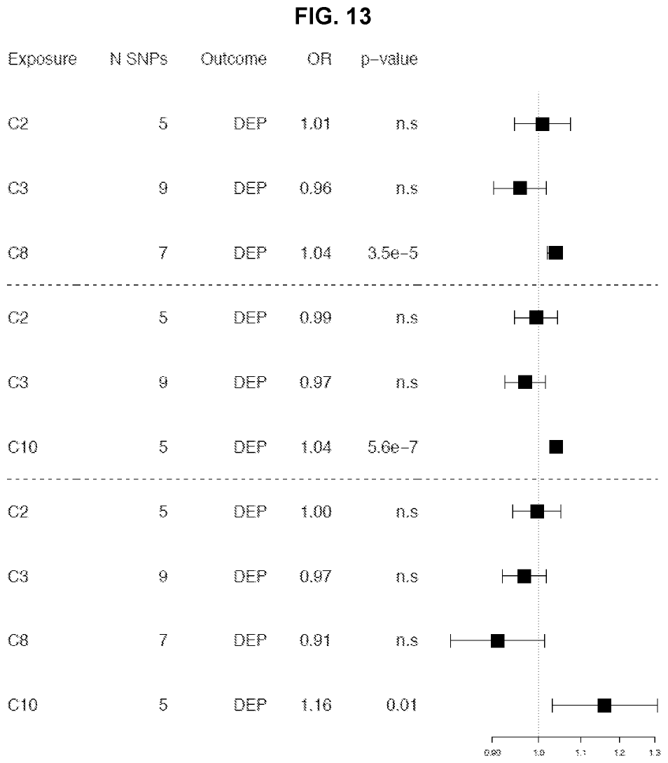

Genetic instruments for C2, C3, C8 and C10 were applied in MVMR analyses conditioning the effect of each exposure instrument on the others. Because the C8-C10 genetic correlation was equal to 1, it had alternatively included them in two initial models with C2 and C3 (FIG. 13, upper and middle panel). Both models showed that only genetically-predicted higher levels of the medium-chain ACs, C8 (OR=1.04, 95% CIs=1.02-1.06, p=3.5×10⁻⁵) or C10 (OR=1.04, 95% CIs=1.02-1.06, p=5.6×10⁻⁷), were similarly associated with higher risk of DEP. In a final model including all genetic instruments (FIG. 12, lower panel) only that of C10 remained significantly associated (OR=1.16, 95% CIs=1.03-1.31, p=0.01) with DEP risk.

In the present study, the potential causal role of ACs metabolism in depression was examined using novel genetic data and Mendelian randomization analyses. Genetically-predicted higher levels of the short-chain acetylcarnitine (C2) and propionylcarnitine (C3) were inversely associated with the risk of depression, while genetically-predicted higher levels of the medium-chain octanoylcarnitine (C8) and decanoylcarnitine (C10) were associated with an increased depression risk. In multivariable analyses, the association with higher depression risk was mainly driven by genetic instruments indexing higher levels of medium-chain ACs. Furthermore, no evidence was found for a potential reversed association between depression liability as exposure and ACs levels as outcome.

Findings from the present study showed that circulating ACs are substantially influenced by genetics, with common SNPs explaining 14-30% of AC levels variance (SNP-heritability). This is consistent with previous studies showing large heritability estimates for these metabolites despite the limited number of loci associated, a pattern reflecting a sparse genetic architecture with few biologically relevant loci. Furthermore, the different ACs shared a substantial proportion their genetic liability, as indicated by moderate (across chain lengths) to strong (within short, medium, and long chain lengths) genetic correlations between ACs. These patterns of genetic overlap are in line with genetic associations results replicated across many mGWAS studies. Previous studies have reported chain length-unspecific associations at loci harboring ACs transporters, such as the organic cation transporters SLC22A4 and SLC22A5, or co-enzymes involved in beta-oxidation, such as ETFDH. In contrast, enzymes with narrower substrate specificity, such as the short-, medium-, and long-chain acyl-CoA dehydrogenases ACADS, ACADM, and ACADL that are involved in beta-oxidation, show more specific association patterns for the respective AC species, resulting in the stronger genetic correlations observed here.

The main findings of the present study suggest that higher levels of medium-chain ACs, or the underlying mechanism responsible for their heightened production, are potentially causal for the development of depression. In a broader perspective, the present findings are consistent with the hypothesis implicating mitochondrial energetic dysfunctions in the pathophysiology of depression. Previous studies have shown evidence of impaired mitochondrial respiration and energy output in peripheral tissues of patients with depression. ACs exert a key role in the mitochondria, transporting fatty acids for beta-oxidation and energy production. Plasma or serum ACs levels can be used as biomarkers tagging abnormalities in beta-oxidation and inborn errors of metabolism such as MCAD (Medium-chain acyl-coenzyme A dehydrogenase) deficiency, whose clinical screening includes the assessment of potentially elevated levels of octanoylcarnitine (C8) and decanoylcarnitine (C10). Intriguingly, clinical manifestations of fatty acid oxidation disorders include neuropsychological and behavioral symptoms present in depression and other psychiatric disorders, such as cognitive impairments, mood alterations, irritability, sleep and appetite dysregulations and low energy.

Cellular energetic dysfunctions may be linked to depression through several pathways. In the brain, altered cellular metabolism may lead to neurotoxicity and impaired neuroplasticity. In animal models, supplementation with acetylcarnitine (C2), for which higher genetically-predicted concentrations were associated with a reduced risk of depression in the present univariable MR analyses, has been shown to promote neuroplasticity and neurotrophic factor synthesis, to modulate glutamatergic dysfunction and related neuronal atrophy in the hippocampus and amygdala, and to ameliorate depression-like behavioral phenotypes. Furthermore, mitochondrial dysfunctions may determine immune system alterations linked to depression. Oxidative stress may trigger the activation of the immune system innate branch by stimulating the release of pro-inflammatory cytokines that, in turn, further stimulate oxidative stress (“autotoxic loop”) and participate in depression pathophysiological processes such as alterations in monoaminergic neurotransmission, tryptophan degradation towards neurotoxic end-products, glutamate-related increased excitotoxicity, decreased neurotrophic factors synthesis or hypothalamic-pituitary-adrenal-axis activity disruption. Furthermore, energy dysfunctions in immune cells may explain the impairments in acquired immunity often observed in depression. Interestingly, recent data(44) on T cells of MDD patients showed an impaired metabolic profile accompanied by increased expression of CTP1a (carnitine palmitoyltransferase 1A), the enzyme converting fatty acyl-CoA into ACs. Studies based on animal models showed that increased expression of CTP1a are determined by exposure to chronic stress and may trigger hyperphagia with consequent weight gain, symptoms of so-called “atypical” depression, hyperglycemia, and insulin resistance. Furthermore, cellular energy dysfunction has been linked to cardiometabolic conditions which, in turn, increase the risk of depression. For instance, elevated medium-chain ACs including octanoylcarnitine (C8) have been found in gestational and Type-2 diabetes.

Beyond direct causal mechanisms, an association between ACs metabolism and depression may arise from distal common environmental (e.g., exposure to stressful life events) or lifestyle (sedentariness, smoking, consumption of high-fat diet or alcohol) factors, which could also represent behavioral consequences of depression. Nevertheless, estimates derived in the present study via MR, suggesting a potential causal role of ACs metabolism in the development of depression, are unlikely to be significantly biased by confounding factors. Furthermore, no evidence was found for a potential reversed causal role of depression in influencing ACs levels. Other implicit properties of MR need to be considered when interpreting the present findings. Genetic variants index the average lifetime exposure to ACs levels and, consequently, MR estimates derived from these instruments describe a potential average lifetime causal effect of ACs on depression and thus are unable to identify specific critical windows or acute events. Furthermore, since genetic instruments for depression were derived from large case-control GWAS, MR results provide information on potential causal mechanisms of disease onset rather than progression, which could involve different pathophysiological pathways. Finally, the causal relationship between ACs and depression identified in the present study requires confirmation across multiple causally-oriented methodologies (e.g., triangulation), including further experimental and mechanistic studies in animals and humans.

A major strength of the present study is the application of 2SMR analyses leveraging novel GWAS summary statistics obtained from the largest international consortia. Nevertheless, despite the use of the largest available input data, the lack of statistically significant causal estimates may have resulted from genetic instruments being insufficiently powered. Biologically meaningful genetic instruments for ACs had adequate strength but were available in low number. In contrast, a higher number of genetic instruments for depression were available but are consistent with a polygenic architecture of weak effects scattered across the genome typical of all behavioral traits. Another limitation is that genetic instruments were derived from GWAS based on samples of European ancestry and therefore results cannot be generalized to different populations. Thus, the present findings should be re-evaluated in the future when results of larger and more ancestry-diverse GWAS will become available. A final limitation is that proper deconvolution of depression heterogeneity was not possible, which may aggregate different dimensions characterized by distinct pathophysiology. Previous studies showed that inflammatory and metabolic alterations, potentially related to mitochondrial dysfunctions, maps more consistently to depressive “atypical” symptoms characterized by altered energy intake/expenditure balance (e.g., hyperphagia, weight gain, hypersomnia, fatigue, leaden paralysis) and anhedonia. Future studies in large cohorts with deeper phenotypes should investigate whether the genetic signature of ACs is differentially associated with various depression clinical features and symptom profiles.

In conclusion, evidence of the potential causal role of ACs dysregulations on the risk of depression, in particular of high levels of medium-chain C8 and C10 was found. Accumulation of medium-chain ACs is a signature of inborn errors of metabolism and age-related metabolic conditions. This suggests that altered cellular energy production and mitochondrial dysfunctions may have a role in depression pathophysiology. Acylcarnitine metabolism represents a promising access point to depression pathophysiology for novel therapeutic approaches.

### Example 5

Association of Plasma and CSF Cytochrome P450, Soluble Epoxide Hydrolase and Ethanolamides Metabolism with Alzheimer's Disease

Subjects: All participants from whom plasma and CSF samples were collected provided informed consent under protocols approved by the Institutional Review Board at Emory University. Cohorts included the Emory Healthy Brain Study (IRB00080300), Cognitive Neurology Research (IRB00078273), and Memory at Emory (IRB00079069). All protocols were reviewed and approved by the Emory University Institutional Review Board. All patients received standardized cognitive assessments (including Montreal Cognitive Assessment (MoCA)) in the Emory Cognitive Neurology clinic, the Emory Goizueta Alzheimer's Disease Research Center (ADRC) and affiliated Emory Healthy Brain Study (EHBS). All diagnostic data were supplied by the ADRC and the Emory Cognitive Neurology Program. CSF was collected by lumbar puncture and banked according to 2014 ADC/NIA best practices guidelines. All CSF samples collected from research participants in the ADRC, Emory Healthy Brain Study, and Cognitive Neurology clinic were assayed using the INNO-BIA AlzBio3 Luminex assay at AKESOgen (Peachtree Corners, GA). AD cases and healthy individuals were defined using established biomarker cutoff criteria for AD for each assay platform. In total, plasma samples were available for 148 AD patients and 133 healthy controls and CSF samples were available for 150 AD patients and 139 healthy controls. Plasma and CSF sample collection overlap (both plasma and CSF collected at the same day) was 145 for AD group and 133 for the control group. Cohort summary statistics for gender, age, MoCA, AB42, tTau, pTau, ApoE genotype and ethnicity are provided in Table 6.

TABLE 6
Cohort Characteristics

| | CSF | | Plasma | |
|---|---|---|---|---|
| | AD | Control | AD | Control |
| | (n = 150) | (n = 139) | (n = 148) | (n = 133) |
| Males:Females | 47%:53% | 28%:72% | 47%:53% | 27%:73% |
| Agea | 68.2 ± 1.34 | 65.2 ± 1.38 | 68 ± 1.36 | 65.6 ± 1.53 |
| AB42ª | 300 ± 17.4 | 536 ± 25.8 | 300 ± 17.4 | 530 ± 25.8 |
| MoCAª | 16.4 ± 1.02 | 26.5 ± 0.523 | 16.4 ± 1.04 | 26.5 ± 0.579 |
| pTauª | 62.8 ± 3.88 | 31.6 ± 2.38 | 62.8 ± 3.93 | 30.7 ± 2.44 |
| tTauª | 115 ± 7.58 | 54.1 ± 4.09 | 115 ± 7.71 | 53.7 ± 4.6 |

ApoE genotype %

| | | | | |
|---|---|---|---|---|
| E2/E3 | 1% | 17% | 1% | 17% |
| E2/E4 | 2% | 5% | 2% | 4% |
| E3/E3 | 27% | 53% | 27% | 55% |
| E3/E4 | 51% | 22% | 50% | 21% |
| E4/E4 | 19% | 3% | 20% | 3% |

Race %

| | | | | |
|---|---|---|---|---|
| Native | 1% | — | 1% | — |
| americans | | | | |
| Asian | 1% | 1% | 1% | — |
| Black or | 7% | 14% | 7% | 14% |
| African | | | | |
| American | | | | |
| Caucasian | 92% | 86% | 92% | 86% |
| or White | | | | |

^{a}Results are means ± 95% confidence intervals

Quantification of lipid mediators: Plasma concentrations of non-esterified PUFA, oxylipins, endocannabinoids, a group of non-steroidal anti-inflammatory drugs (NSAIDs) including ibuprofen, naproxen, acetaminophen, a suite of conjugated and unconjugated bile acids, and a series of glucocorticoids, progestins and testosterone were quantified in 50 μL of plasma by liquid chromatography tandem mass spectrometry (LC-MS/MS) after protein precipitation in the presence of deuterated metabolite analogs (i.e., analytical surrogates). CSF analyses were performed with 100 μL samples prepared as previously reported for the analyses of sweat and analyzed as reported for plasma. All samples were processed with rigorous quality control measures including case/control randomization, and the analysis of batch blanks, pooled matrix replicates and NIST Standard Reference Material 1950—Metabolites in Human Plasma (Sigma-Aldrich, St Louis, MO). Extraction batches were re-randomized for acquisition, with method blanks and reference materials and calibration solutions scattered regularly throughout the set. Instrument limits of detection (LODs) and limits of quantification (LOQs) were estimated according to the Environmental Protection Agency method (40 CFR, Appendix B to Part 136 revision 1.11, U.S. and EPA 821-R-16-006 Revision 2). These values were then transformed into sample nM concentrations by multiplying the calculated concentration by the final sample volume and dividing by the volume of sample extracted. A complete analyte list with plasma LODs and LOQs have been reported. The majority of analytes were quantified against analytical standards with the exception of eicosapentaenoyl ethanolamide (EPEA), palmitoleoyl ethanolamide (POEA), and the measured PUFAs [i.e., linoleic acid (LA); alpha-linolenic acid (aLA); arachidonic acid (AA); eicosapentaenoic acid (EPA); docosahexaenoic acid (DHA)]. For these compounds, area counts were recorded, adjusted for deuterated-surrogate responses and the relative response factors were expressed as the relative abundance across all analyzed samples. Reported monoacylglycerols (MAGs) are the sum of 1- and 2-acyl isomers, due to isomerization during sample processing.

Fasting state assessment and sample selection: Many of the CSF and plasma samples from AD patients were collected following additional research consent in the course of patients' clinical evaluations. Lumbar puncture procedures were nearly all scheduled in the morning but fasting was not mandated in these individuals. Therefore, fasting state of the samples was estimated using previously published predictive equation. A high probability of the fasted state was described by low levels of the LA-derived CYP metabolite [12(13)-EpOME], low levels of the primary conjugated bile acid glycochenodeoxycholic acid (GCDCA) and elevated levels of the glycine-conjugated oleic acid (NO-Gly). Fasting probability was calculated using Equation 1 and Equation 2.

$$Probability for fasted = \frac{1}{( 1 + Exp ( - Lin . prob . fasted ) )} \qquad Equation 1$$

Probability of the fated state. Where “Lin.prob.fasted” is defined by the Equation 2:

Lin.prob.fasted=10.01−(2.82×a)+(1.94×b)−(1.35×c) Equation 2

Lin prob fasted: a=Log[12(13)-EpOME]; b=Log(NO-Gly); c=Log(GCDCA). Concentrations expressed in (nM).

Only subjects with the probability of the fasting state >60% were used for the plasma analysis. All subjects were used to compare lipid mediators level in CSF. CSF was reported not to manifest postprandial lipid fluctuations, additionally, comparing predicted fasted to predicted non-fasted AD subjects reveal minimal differences in only 2 metabolites (Table 7).

TABLE 7
T-test of predicted fasted vs predicted non-fasted in AD group for plasma and CSF
CSF

| | | Fold | Mean (nM) [95% CI] | | | |
|---|---|---|---|---|---|---|
| Metabolite | P value | change | Fasted | | Non-fasted | |
| PGF2a | 0.0251 | 1.25 | 0.0274 | [0.0235-0.032] | 0.0342 | [0.0301-0.0388] |
| GCA/GCDCA | 0.0263 | 1.18 | 1.12 | [0.993-1.27] | 1.32 | [1.15-1.52] |
| 12_13-DiHOME | 0.03 | 1.27 | 0.0305 | [0.0268-0.0347] | 0.0387 | [0.0328-0.0457] |
| UDCA/CDCA | 0.0472 | 0.585 | 0.978 | [0.683-1.4] | 0.572 | [0.441-0.744] |
| 12,13- | 0.0626 | 1.24 | 0.0918 | [0.077-0.109] | 0.114 | [0.0939-0.137] |
| DiHOME/EpOME | | | | | | |
| OEA | 0.07 | 1.13 | 0.082 | [0.0757-0.0888] | 0.0923 | [0.0832-0.102] |
| DCA/(LCA + UDCA) | 0.0737 | 1.52 | 1.83 | [1.28-2.62] | 2.79 | [2.13-3.65] |
| TCA | 0.0742 | 1.38 | 0.407 | [0.295-0.561] | 0.563 | [0.447-0.711] |
| 15_16-DiHODE | 0.0875 | 1.16 | 0.0669 | [0.0567-0.0789] | 0.0775 | [0.0666-0.0902] |
| GCA | 0.0925 | 1.25 | 0.887 | [0.712-1.11] | 1.11 | [0.897-1.37] |
| LA_RelAb | 0.108 | 0.915 | 0.248 | [0.228-0.269] | 0.227 | [0.212-0.244] |
| GLCA/CDCA | 0.114 | 0.674 | 0.0362 | [0.0263-0.0497] | 0.0244 | [0.0187-0.0318] |
| Sum(DiHOME)/ | 0.121 | 1.2 | 0.0794 | [0.066-0.0955] | 0.0954 | [0.0788-0.115] |
| Sum(EpOME) | | | | | | |
| 9_10-DiHOME | 0.122 | 1.17 | 0.0197 | [0.017-0.0228] | 0.023 | [0.0194-0.0273] |
| GDCA/GLCA | 0.14 | 1.34 | 10.1 | [7.44-13.6] | 13.5 | [10.7-17.2] |
| UDCA | 0.153 | 0.693 | 1.12 | [0.767-1.62] | 0.776 | [0.593-1.01] |
| (GDCA + TDCA)/ | 0.154 | 1.33 | 11.4 | [8.4-15.5] | 15.2 | [12-19.4] |
| (TLCA + GLCA) | | | | | | |
| GCDCA/GLCA | 0.169 | 1.32 | 19.2 | [14.8-24.8] | 25.4 | [20.6-31.2] |
| TUDCA/UDCA | 0.189 | 1.46 | 0.00304 | [0.00188-0.00489] | 0.00445 | [0.0031-0.00639] |
| GLCA | 0.226 | 0.799 | 0.0413 | [0.0332-0.0512] | 0.033 | [0.0269-0.0406] |
| EPA + DHA diols | 0.236 | 0.866 | 0.157 | [0.137-0.181] | 0.136 | [0.119-0.155] |
| 17_18-DiHETE | 0.268 | 0.768 | 0.104 | [0.0877-0.124] | 0.0799 | [0.063-0.101] |
| 9,10- | 0.269 | 1.14 | 0.0641 | [0.0517-0.0794] | 0.0728 | [0.0588-0.09] |
| DiHOME/EpOME | | | | | | |
| GCA/GDCA | 0.27 | 1.16 | 2.14 | [1.66-2.75] | 2.48 | [1.96-3.14] |
| 13-HOTE | 0.273 | 1.08 | 0.0478 | [0.0419-0.0546] | 0.0517 | [0.047-0.0569] |
| GUDCA/UDCA | 0.291 | 1.37 | 0.0921 | [0.0617-0.137] | 0.126 | [0.0904-0.176] |
| (TUDAC + GUDCA)/ | 0.295 | 1.36 | 0.0976 | [0.0656-0.145] | 0.133 | [0.0954-0.184] |
| UDCA | | | | | | |
| ALA_RelAb | 0.297 | 0.949 | 0.272 | [0.253-0.293] | 0.258 | [0.24-0.276] |
| (GDCA + GLCA)/ | 0.31 | 0.905 | 11.6 | [9.19-14.6] | 10.5 | [9.13-12.1] |
| (TDCA) | | | | | | |
| 19_20-DiHDoPE | 0.333 | 0.918 | 0.0183 | [0.0157-0.0213] | 0.0168 | [0.0151-0.0188] |
| AA_RelAb | 0.354 | 0.941 | 0.256 | [0.234-0.281] | 0.241 | [0.223-0.261] |
| CDCA | 0.375 | 1.19 | 1.14 | [0.886-1.47] | 1.36 | [1.11-1.65] |
| GCDCA/CDCA | 0.376 | 0.892 | 0.693 | [0.532-0.903] | 0.618 | [0.525-0.727] |
| TCDCA | 0.387 | 1.11 | 0.0656 | [0.051-0.0845] | 0.0726 | [0.0583-0.0903] |
| (TCDCA + GCDCA)/ | 0.396 | 0.889 | 0.767 | [0.587-1] | 0.682 | [0.578-0.805] |
| CDCA | | | | | | |
| a-MCA | 0.4 | 1.22 | 0.0316 | [0.0212-0.047] | 0.0387 | [0.0291-0.0515] |
| 20-HETE | 0.41 | 1.07 | 0.121 | [0.107-0.136] | 0.13 | [0.118-0.143] |
| T-a-MCA | 0.415 | 1.06 | 0.0294 | [0.0229-0.0378] | 0.0311 | [0.0243-0.0399] |
| 13-HODE | 0.416 | 1.03 | 1.56 | [1.51-1.61] | 1.61 | [1.55-1.68] |
| EPA_RelAb | 0.418 | 0.915 | 0.211 | [0.179-0.25] | 0.193 | [0.168-0.222] |
| TCDCA/GCDCA | 0.421 | 1.04 | 0.083 | [0.0683-0.101] | 0.0867 | [0.0742-0.101] |
| GDCA | 0.427 | 1.08 | 0.415 | [0.325-0.531] | 0.447 | [0.364-0.549] |
| 14,15/11,12- | 0.428 | 1.03 | 2.74 | [2.59-2.9] | 2.82 | [2.67-2.98] |
| DiHETrE | | | | | | |
| 9-HOTE | 0.428 | 1.05 | 0.0119 | [0.0106-0.0133] | 0.0125 | [0.0112-0.0139] |
| 14_15-DiHETE | 0.431 | 0.874 | 0.0247 | [0.0196-0.0312] | 0.0216 | [0.0168-0.0279] |
| TDCA | 0.432 | 1.14 | 0.0422 | [0.0303-0.0588] | 0.0479 | [0.0377-0.0609] |
| TEST | 0.434 | 1.11 | 0.0426 | [0.0301-0.0602] | 0.0472 | [0.0358-0.0623] |
| 17OH-PROG | 0.436 | 1.1 | 0.0436 | [0.0381-0.0498] | 0.0479 | [0.0424-0.0541] |
| 11_12-DiHETrE | 0.45 | 0.954 | 0.0175 | [0.0154-0.0199] | 0.0167 | [0.0151-0.0184] |
| w-MCA/UDCA | 0.456 | 1.34 | 0.107 | [0.071-0.162] | 0.143 | [0.0968-0.211] |
| w-MCA/T-a-MCA | 0.528 | 0.875 | 4.07 | [2.54-6.53] | 3.56 | [2.23-5.67] |
| TDCA/GDCA | 0.57 | 1.05 | 0.102 | [0.0823-0.126] | 0.107 | [0.0937-0.123] |
| (TDCA + TCDCA)/ | 0.587 | 1.03 | 0.0934 | [0.0775-0.113] | 0.0958 | [0.0837-0.11] |
| (GDCA + GCDCA) | | | | | | |
| LEA | 0.59 | 1 | 0.255 | [0.237-0.274] | 0.256 | [0.245-0.268] |
| DHEA | 0.592 | 0.939 | 0.0163 | [0.0141-0.0188] | 0.0153 | [0.0138-0.0171] |
| DCA | 0.609 | 1.06 | 2.04 | [1.58-2.64] | 2.16 | [1.7-2.75] |
| (GDCA + TDCA)/ | 0.62 | 1.13 | 4.33 | [2.96-6.32] | 4.89 | [3.65-6.54] |
| (TUDCA + GUDCA) | | | | | | |
| TDCA/DCA | 0.626 | 1.07 | 0.0207 | [0.0144-0.0296] | 0.0222 | [0.0174-0.0283] |
| TDCA/DCA 2 | 0.626 | 1.07 | 0.0207 | [0.0144-0.0296] | 0.0222 | [0.0174-0.0283] |
| w-MCA | 0.634 | 0.925 | 0.12 | [0.0816-0.176] | 0.111 | [0.0778-0.158] |
| CRTN | 0.701 | 1.03 | 4.26 | [3.89-4.66] | 4.37 | [4.1-4.66] |
| GCDCA/GDCA | 0.744 | 0.984 | 1.9 | [1.52-2.39] | 1.87 | [1.51-2.32] |
| 11-Deoxy-CTRL | 0.746 | 0.948 | 0.0541 | [0.0474-0.0617] | 0.0513 | [0.0454-0.058] |
| GUDCA | 0.769 | 0.949 | 0.103 | [0.0766-0.138] | 0.0977 | [0.0771-0.124] |
| TUDCA | 0.769 | 1.02 | 0.00339 | [0.0025-0.00459] | 0.00345 | [0.00271-0.0044] |
| F2-IsoP | 0.79 | 0.97 | 0.301 | [0.28-0.323] | 0.292 | [0.272-0.314] |
| CRTL | 0.855 | 1 | 14.4 | [13.1-15.9] | 14.4 | [13.5-15.4] |
| 12(13)-EpOME | 0.863 | 1.03 | 0.332 | [0.285-0.387] | 0.341 | [0.298-0.391] |
| TCDCA/CDCA | 0.887 | 0.932 | 0.0575 | [0.0402-0.0822] | 0.0536 | [0.0412-0.0696] |
| GCDCA | 0.889 | 1.06 | 0.79 | [0.658-0.949] | 0.837 | [0.723-0.97] |
| 14_15-DiHETrE | 0.901 | 0.981 | 0.0479 | [0.043-0.0535] | 0.047 | [0.0435-0.0508] |
| DHA_RelAb | 0.925 | 0.988 | 0.256 | [0.224-0.293] | 0.253 | [0.224-0.286] |
| 9-HODE | 0.931 | 1.02 | 0.532 | [0.511-0.553] | 0.544 | [0.518-0.571] |
| GDCA/DCA | 0.934 | 1.02 | 0.203 | [0.166-0.249] | 0.207 | [0.179-0.239] |
| (TDCA + GDCA)/ | 0.937 | 1.01 | 0.231 | [0.186-0.286] | 0.233 | [0.2-0.271] |
| DCA | | | | | | |
| CRCTN | 0.954 | 0.998 | 0.414 | [0.35-0.49] | 0.413 | [0.361-0.472] |
| 9(10)-EpOME | 0.975 | 1.03 | 0.307 | [0.257-0.366] | 0.317 | [0.268-0.374] |
| T-a-MCA/CDCA | 0.995 | 0.891 | 0.0258 | [0.0181-0.0368] | 0.023 | [0.0164-0.0322] |
| Plasma | | | | | | |
| 12(13)-EpOME | 0.0001 | 2.79 | 2.41 | [2.07-2.8] | 6.73 | [5.54-8.17] |
| 12_13-DiHODE | 0.0001 | 2.52 | 0.209 | [0.161-0.271] | 0.527 | [0.448-0.62] |
| 12_13-DiHOME | 0.0001 | 2.29 | 3.86 | [3.38-4.41] | 8.83 | [7.62-10.2] |
| 13-HOTE | 0.0001 | 2.26 | 0.624 | [0.483-0.806] | 1.41 | [1.17-1.7] |
| 15(16)-EPODE | 0.0001 | 2.17 | 2.42 | [2.06-2.84] | 5.24 | [4.33-6.35] |
| 15_16-DiHODE | 0.0001 | 2.28 | 10.9 | [9.47-12.5] | 24.9 | [21.5-28.9] |
| 9(10)-EpOME | 0.0001 | 1.92 | 0.372 | [0.305-0.454] | 0.713 | [0.574-0.885] |
| 9_10-DiHODE | 0.0001 | 4.33 | 0.183 | [0.144-0.233] | 0.792 | [0.629-0.997] |
| 9_10-DiHOME | 0.0001 | 2.93 | 3.25 | [2.94-3.59] | 9.53 | [8.04-11.3] |
| 9_10-e-DiHO | 0.0001 | 1.56 | 3.05 | [2.68-3.46] | 4.75 | [4.07-5.54] |
| 9-HOTE | 0.0001 | 1.72 | 0.446 | [0.39-0.511] | 0.769 | [0.652-0.906] |
| AA_screen | 0.0001 | 0.645 | 0.155 | [0.139-0.172] | 0.1 | [0.0895-0.113] |
| AEA | 0.0001 | 0.691 | 1.49 | [1.36-1.64] | 1.03 | [0.92-1.15] |
| GCA | 0.0001 | 3.31 | 68.6 | [51.6-91.1] | 227 | [179-288] |
| GCDCA | 0.0001 | 2.97 | 367 | [270-499] | 1090 | [896-1330] |
| GDCA | 0.0001 | 2.83 | 186 | [125-277] | 526 | [399-694] |
| NA-Gly | 0.0001 | 0.619 | 0.72 | [0.621-0.835] | 0.446 | [0.389-0.512] |
| NO-Gly | 0.0001 | 0.53 | 4.7 | [4.29-5.16] | 2.49 | [2.18-2.85] |
| OEA | 0.0001 | 0.755 | 5.23 | [4.79-5.7] | 3.95 | [3.58-4.36] |
| TCA | 0.0001 | 3.56 | 11.7 | [8.1-16.9] | 41.7 | [32.3-53.9] |
| TCDCA | 0.0001 | 3.27 | 30.3 | [21.6-42.7] | 99 | [77.4-127] |
| TDCA | 0.0001 | 2.92 | 13.7 | [8.95-21] | 40 | [29.1-55.1] |
| POEA_Screen | 0.0002 | 0.603 | 0.119 | [0.0985-0.144] | 0.0718 | [0.0602-0.0856] |
| TLCA | 0.0002 | 2.3 | 7.56 | [5.18-11.1] | 17.4 | [13.2-23] |
| DHA_screen | 0.0003 | 0.652 | 0.149 | [0.128-0.174] | 0.0971 | [0.0838-0.113] |
| DHEA | 0.0003 | 0.726 | 1.18 | [1.04-1.34] | 0.857 | [0.78-0.941] |
| LA_screen | 0.0004 | 0.722 | 0.158 | [0.138-0.18] | 0.114 | [0.101-0.129] |
| 9_12_13-TriHOME | 0.0005 | 1.63 | 2.65 | [2.2-3.18] | 4.32 | [3.64-5.13] |
| GUDCA | 0.0006 | 2.31 | 46.3 | [32.8-65.4] | 107 | [80.8-142] |
| LEA | 0.0006 | 0.811 | 4.5 | [4.13-4.91] | 3.65 | [3.39-3.93] |
| ALA_screen | 0.0008 | 0.646 | 0.153 | [0.128-0.184] | 0.0988 | [0.0822-0.119] |
| GLCA | 0.0009 | 2.27 | 21.8 | [14.8-32.1] | 49.5 | [38.2-64.1] |
| 9-HODE | 0.0015 | 1.46 | 10.9 | [9.66-12.2] | 15.9 | [13.5-18.7] |
| DGLEA | 0.0016 | 0.726 | 0.175 | [0.151-0.201] | 0.127 | [0.11-0.147] |
| EPA_screen | 0.0017 | 0.625 | 0.13 | [0.108-0.156] | 0.0813 | [0.0672-0.0983] |
| 13-HODE | 0.0023 | 1.45 | 14.3 | [12.7-16.1] | 20.7 | [17.5-24.5] |
| GHDCA | 0.0023 | 1.88 | 0.8 | [0.611-1.05] | 1.5 | [1.24-1.82] |
| T-a-MCA | 0.0037 | 1.95 | 2.62 | [1.9-3.63] | 5.1 | [3.86-6.73] |
| 5-HETE | 0.0056 | 0.755 | 3.79 | [3.35-4.29] | 2.86 | [2.52-3.23] |
| DEA | 0.0072 | 0.788 | 0.378 | [0.325-0.44] | 0.298 | [0.266-0.334] |
| TUDCA | 0.0084 | 1.69 | 1.92 | [1.54-2.4] | 3.24 | [2.56-4.09] |
| 12-HETE | 0.0137 | 0.786 | 2.81 | [2.46-3.21] | 2.21 | [1.95-2.51] |
| 15-HETE | 0.0202 | 0.824 | 3.13 | [2.81-3.49] | 2.58 | [2.28-2.92] |
| Ibuprofen | 0.0266 | 0.427 | 41 | [21.4-78.4] | 17.5 | [10.9-27.9] |
| w-MCA | 0.0392 | 1.78 | 7.68 | [5.53-10.7] | 13.7 | [10.2-18.4] |
| CDCA | 0.0401 | 1.9 | 46.7 | [32.6-66.9] | 88.5 | [61.6-127] |
| 1-LG | 0.0436 | 1.23 | 175 | [155-197] | 215 | [192-241] |
| aLEA | 0.0483 | 0.837 | 0.184 | [0.164-0.207] | 0.154 | [0.138-0.172] |
| DCA | 0.0893 | 1.55 | 177 | [120-259] | 274 | [206-366] |
| TEST | 0.0953 | 0.924 | 2.5 | [1.63-3.85] | 2.31 | [1.6-3.34] |
| 11_12-DiHETrE | 0.0989 | 0.877 | 0.81 | [0.735-0.894] | 0.71 | [0.64-0.788] |
| 11-HETE | 0.1022 | 0.845 | 1.61 | [1.4-1.86] | 1.36 | [1.19-1.56] |
| 9(10)-EpODE | 0.1086 | 1.32 | 0.145 | [0.112-0.187] | 0.192 | [0.146-0.253] |
| 9-HETE | 0.1162 | 0.803 | 1.32 | [1.12-1.57] | 1.06 | [0.894-1.27] |
| 2-LG | 0.1187 | 1.19 | 46.4 | [41-52.5] | 55 | [49.4-61.3] |
| 13-KODE | 0.1265 | 0.732 | 1.42 | [1.16-1.74] | 1.04 | [0.809-1.33] |
| 11(12)-EpETrE | 0.1495 | 0.697 | 0.0755 | [0.0608-0.0938] | 0.0526 | [0.0415-0.0665] |
| 14(15)-EpETrE | 0.1565 | 1.07 | 0.106 | [0.0826-0.136] | 0.113 | [0.0936-0.137] |
| PGF2a | 0.1565 | 1.35 | 0.416 | [0.313-0.554] | 0.56 | [0.473-0.664] |
| 14-HDoHE | 0.1646 | 0.804 | 1.58 | [1.19-2.11] | 1.27 | [0.96-1.67] |
| CA | 0.1729 | 1.66 | 19.3 | [13.6-27.4] | 32 | [21.7-47.2] |
| Progesterone | 0.1736 | 1.26 | 0.37 | [0.317-0.432] | 0.466 | [0.39-0.558] |
| EPEA_Screen | 0.1844 | 0.808 | 0.0873 | [0.0704-0.108] | 0.0705 | [0.0581-0.0855] |
| 14_15-DiHETrE | 0.2208 | 0.922 | 1 | [0.912-1.1] | 0.922 | [0.83-1.02] |
| 8_9-DiHETrE | 0.249 | 0.742 | 0.365 | [0.279-0.476] | 0.271 | [0.215-0.342] |
| PGE2 | 0.2637 | 1.22 | 0.16 | [0.13-0.197] | 0.195 | [0.16-0.238] |
| 5_6-DiHETrE | 0.2763 | 0.875 | 0.51 | [0.449-0.581] | 0.446 | [0.384-0.518] |
| Naproxen | 0.3047 | 0.565 | 191 | [57.1-637] | 108 | [40-294] |
| 5-HEPE | 0.3191 | 0.82 | 0.572 | [0.463-0.706] | 0.469 | [0.387-0.568] |
| b-MCA | 0.3361 | 0.813 | 2.41 | [1.79-3.24] | 1.96 | [1.55-2.49] |
| 12-HEPE | 0.4055 | 0.9 | 0.311 | [0.252-0.383] | 0.28 | [0.236-0.332] |
| Acetaminophen | 0.4853 | 1.11 | 5.52 | [2.81-10.8] | 6.14 | [3.36-11.2] |
| 1-AG | 0.5174 | 1.07 | 19 | [16.9-21.4] | 20.4 | [18.2-22.8] |
| UDCA | 0.5175 | 0.877 | 91.6 | [62.8-134] | 80.3 | [59.2-109] |
| LCA | 0.5555 | 1.12 | 30 | [23.7-38] | 33.6 | [27.2-41.5] |
| 9-HEPE | 0.5787 | 1.02 | 0.158 | [0.111-0.225] | 0.161 | [0.132-0.196] |
| 1-OG | 0.595 | 1.09 | 282 | [252-316] | 308 | [276-344] |
| 2-AG | 0.6627 | 1.07 | 4.77 | [4.24-5.37] | 5.12 | [4.49-5.84] |
| CRCTN | 0.6773 | 1.09 | 0.743 | [0.58-0.953] | 0.808 | [0.693-0.943] |
| CRTN | 0.6992 | 0.966 | 4.76 | [3.71-6.11] | 4.6 | [3.85-5.49] |
| 4-HDoHE | 0.717 | 0.951 | 0.649 | [0.549-0.768] | 0.617 | [0.506-0.751] |
| 17_18-DiHETE | 0.7633 | 1.01 | 5.23 | [4.32-6.34] | 5.29 | [4.49-6.23] |
| 15-HEPE | 0.7908 | 0.925 | 0.292 | [0.239-0.356] | 0.27 | [0.223-0.327] |
| 8-HETE | 0.8305 | 0.992 | 2.51 | [2.16-2.93] | 2.49 | [2.18-2.84] |
| TXB2 | 0.8681 | 1.03 | 0.231 | [0.183-0.292] | 0.239 | [0.193-0.298] |
| 19_20-DiHDoPE | 0.8845 | 0.985 | 2.03 | [1.78-2.33] | 2 | [1.8-2.23] |
| 17-OH PROG | 0.8963 | 1.03 | 0.951 | [0.764-1.18] | 0.978 | [0.805-1.19] |
| PGD2 | 0.9007 | 0.928 | 0.264 | [0.176-0.397] | 0.245 | [0.178-0.338] |
| CRTL | 0.9039 | 1.17 | 207 | [158-272] | 242 | [217-270] |
| 2-OG | 0.9312 | 1.05 | 34.1 | [29.8-39.1] | 35.9 | [31.6-40.7] |
| 5_15-DiHETE | 0.9689 | 1.1 | 0.105 | [0.0756-0.146] | 0.115 | [0.0904-0.146] |
| DHEAS | 0.9727 | 1.24 | 1220 | [923-1610] | 1510 | [1310-1740] |
| F2-IsoP | 0.9741 | 0.959 | 6.36 | [5.33-7.59] | 6.1 | [5.34-6.98] |

Statistical analysis: All statistical tests were performed using JMP Pro 14 (JMP, SAS institute, Cary, NC). Prior to analysis, data were tested for outliers using the robust Huber M test and missing data were imputed using multivariate normal imputation for variables which were at least 75% complete. The imputed numbers constitute less than 3% of the data for both plasma and CSF. Imputation facilitated multivariate data analysis and non-imputed data were used for univariate approaches. Additionally, variables were normalized, centered, and scaled using Johnson's transformation, with normality verification using the Shapiro-Wilk test. The difference between the control and the AD group were assessed using t-test with gender, age, and race as covariates. Additionally, two-way ANOVA was used to test for the gender x group and race x group interactions. In case of significant interaction, the group effect was tested separately for the interacting factor. Correlations between MoCA score and lipid mediators were assessed using Spearman's rank order correlation, to account for non-linear associations. This analysis was performed using only AD subjects, stratified by the assessed fasting state for plasma. CSF samples were analyzed without fasting state stratification. Multiple comparison control was accomplished with the false discovery rate (FDR) correction method of Benjamini and Hochberg with a q=0.2. Predictive models for AD were prepared using a combination of bootstrap tree and stepwise logistic regression modeling. Prior to analysis, subjects were randomly split into training (70%) and validation (30%) cohorts. Variables most frequently appearing in the models were identified by bootstrap tree: Number of layers=50; split per tree=3, bootstrap sampling rate for variables and subjects=1. A variable contribution scree plot was generated using variable rank and the likelihood ratio of chi-square. The scree plot was used to determine a likelihood ratio of chi-square cutoff value for variables contributing to the model. Selected variables were then subjected to stepwise logistic regressions. A stepwise analysis was performed with the maximal validation r² as the model stopping criteria, or if an additional step increased the Bayesian information criteria (BIC). Variables selected by the stepwise approach were then used to build the model using logistic regression. Metabolites that the model contribution p-value <0.05 were excluded, to ensure the strongest model with the minimal number of predictors. Partial least square discriminant analysis (PLA-DA) was used to integrate AD related differences in metabolite levels between plasma and CSF. PLS-DA model was built using the nonlinear iterative partial least squares algorithm with K-Fold variation method (k=7) and included 235 variables from plasma and CSF, including metabolite levels and informative metabolite ratios. For clarity purposes, only variables with a variable importance in projection (VIP) score >1.4 were displayed on the loading plot.

Correlation between CSF and plasma metabolites was assessed using Spearman's rank order correlation.

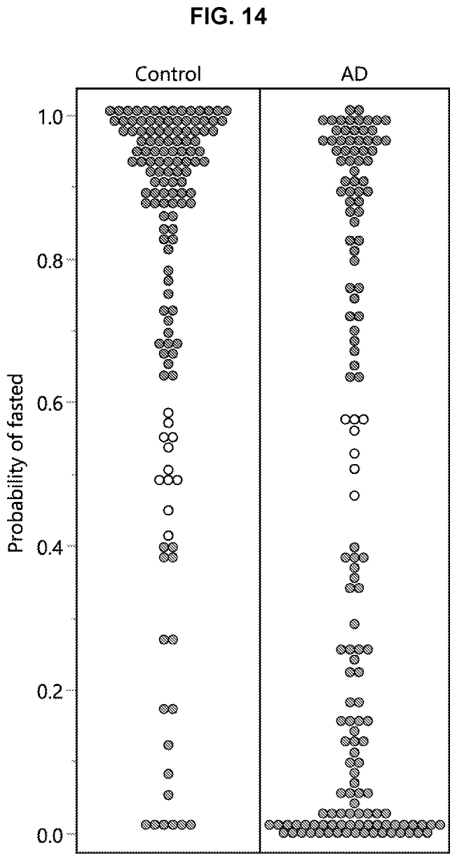

Fasting state assessment (Epoxy linoleate, glycine-conjugated oleic acids, and bile acids): Analysis of opportunistically collected samples brings a challenge of the unknown fasting state. The control cohort contained samples collected in the fasted state per ADRC and EHBS protocols, but the AD cohort included many who had no collected fasting state information and consist of samples collected in both fasted and non-fasted states. Therefore, to allow a direct comparison of the control and AD groups, the estimated subject fasting state was assessed using a previously published predictive model. It should be noted that due to natural variation in postprandial metabolism, the fasting state predictions used here should not be considered as a binary classification (i.e. either fasted or non-fasted), but rather as a 3 group set consisting of a classical fasted profile, a classical non-fasted profile, and a mixed group of “low”-fasted profiles and “high”-non-fasted profiles that cannot be distinguished. As expected, control group was predicted to contain mostly fasted subjects (FIG. 14). Out of 133 control subjects, 105 (i.e., 79%) were predicted as fasted, while 17 (i.e., 12%) as non-fasted, with a probability of >60% and 11 (i.e., 8%) had a fasting state probability of <60%. Out of 148 AD subjects, 60 (i.e., 40%) were predicted as fasted and 81 (i.e., 55%) as non-fasted, with a probability of >60% and 7 (5%) had a fasting state probability of <60%. Fifty percent of detected metabolites manifested difference between predicted fasted and non-fasted AD subjects in plasma and only minimal differences were observed in CSF (Table 7). These differences in plasma metabolites agreed with the published consensus regarding postprandial fluctuation in key metabolites, including fatty acids and bile acids.

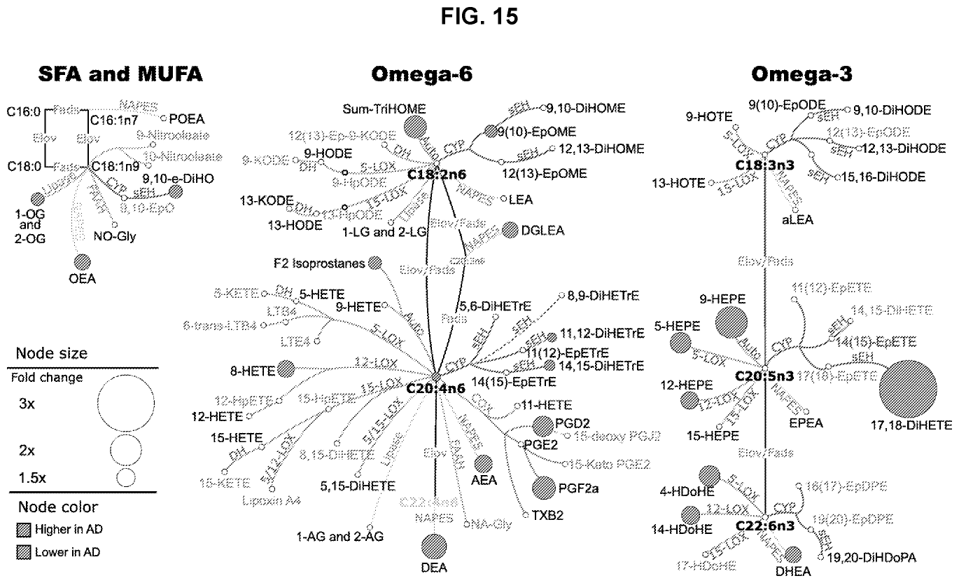

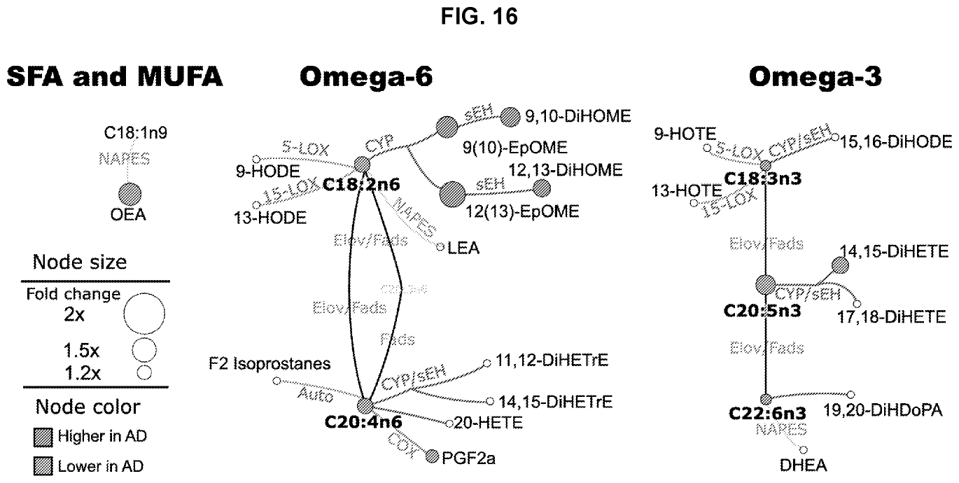

Cytochrome P450/soluble epoxide hydrolase metabolism is elevated in AD subjects (Hydroxy and dihydroxy fatty acids, prostaglandins, and fatty acids ethanolamides): Plasma and CSF lipid mediator concentrations were compared between the control and the AD groups, using only estimated fasted subjects with probability>60% for plasma. In plasma, 42 oxylipins (85 measured), 5 PUFAs (5 measured), 17 endocannabinoids (22 measured), 3 NSAIDs (4 measured), 19 bile acids (23 measured) and 8 steroid hormones (8 measured) were detected. The mean values and p-values for t-tests and two-way ANOVA interactions for all detected metabolites are provided in Table 8. Plasma group-fold differences in the oxylipin, endocannabinoids and PUFAs, projected onto their metabolic pathway, are presented in FIG. 15. The largest differences were observed in the long chain omega-3 PUFA metabolism. Both EPA and DHA enzyme derived mono-alcohols (5-LOX-derived 5-HEPE and 4-HDoHE and 12-LOX-derived 12-HEPE and 14-HDoHE) were lower (1.5-fold in average) in the AD group, when compared to the control. On the other hand, the sEH EPA metabolite 17,18-DiHETE, was 3-fold higher in the AD group. In the AD group, the AA pathway manifested lower levels of the COX derived prostaglandins PGF2α and PGD2 (1.6-fold average). Additionally, the AD group showed lower levels of acylethanolamides (1.5-fold in average) derived from dihomo-gamma-linolenic acid (DGLEA), AA (AEA), docosatetraenoic acid (DEA), DHA (DHEA) and oleic acid (OEA). Notable are also lower levels of autooxidation markers, particularly the EPA-derived 9-HEPE (2-fold), linoleic acid (LA)-derived TriHOMEs (1.65-fold) and AA-derived isoprostanes (1.3-fold) in AD group. Fewer lipid mediators were detected in CSF than in plasma. Detected CSF lipid mediators included 17 oxylipins, 5 PUFAs, 3 endocannabinoids, 14 bile acids and 6 steroids. The mean values and p-values for t-tests and two-way ANOVA interactions are provided in Table 9. CSF significant group-fold differences in the level of oxylipin, endocannabinoids and PUFAs, projected onto their metabolic pathway, are presented in FIG. 16. In this matrix, the largest differences were observed in the LA CYP metabolic pathway, where both epoxy and dihydroxy FA, products of CYP and subsequent sEH metabolism, were higher in the AD group when compared to the control: epoxide average 1.5-fold; diol average 1.3-fold. All PUFAs from both omega-3 and omega-6 pathway were lower in the AD group, although the difference was only 1.2-fold on average. Additionally, AD group manifested 1.5-folds lower level of OEA and 1.3-fold lower level of the EPA-derived 14,15-DiHETE.

TABLE 8
The mean values and t-test and two-way ANOVA interaction
p-values for all detected metabolites in plasma.

| | | | | p-values | | | |
|---|---|---|---|---|---|---|---|
| | | | | Using subjects with p of | | | |
| | | | | fasted >60% | | | |
| | | | | | | p-value | p-value |
| | | | | | p-value | Race × | AD vs |
| Metabolite information | | | | p-value | Gender × | Disease | Control |
| | Chemical | | | AD vs | Disease | inter- | All |
| Metabolite name | class | Enzyme | Units | Control | interaction | action | subjects |

Oxylipins, endocannabinoids, PUFAS, and NSAIDS

| | | | | | | | |
|---|---|---|---|---|---|---|---|
| TXB2 | TX | COX1 | nM | 0.108 | 0.516 | 0.309 | 0.0435 |
| PGE2 | PGs | COX2 | nM | 0.112 | 0.275 | 0.236 | 0.0459 |
| PGD2 | PGs | COX2 | nM | 0.0346 | 0.327 | 0.0459 | 0.0037 |
| PGF2a | PGs | COX2 | nM | 0.0163 | 0.481 | 0.609 | 0.0632 |
| F2-IsoP | PGs | Auto-ox | nM | 0.0059 | 0.407 | 0.923 | 0.0001 |
| 5_15-DiHETE | Diol | LOX | nM | 0.272 | 0.278 | 0.381 | 0.383 |
| 9_12_13-TriHOME | Triol | LOX | nM | 0.0006 | 0.828 | 0.165 | 0.0618 |
| 9_10-e-DiHO | vic-Diol | SEH | nM | 0.0236 | 0.905 | 0.605 | 0.986 |
| 12_13-DiHOME | vic-Diol | SEH | nM | 0.103 | 0.7 | 0.495 | 0.0761 |
| 9_10-DiHOME | vic-Diol | SEH | nM | 0.413 | 0.442 | 0.377 | 0.0001 |
| 15_16-DiHODE | vic-Diol | SEH | nM | 0.813 | 0.858 | 0.849 | 0.0001 |
| 12_13-DiHODE | vic-Diol | SEH | nM | 0.711 | 0.26 | 0.502 | 0.0001 |
| 9_10-DiHODE | vic-Diol | SEH | nM | 0.909 | 0.587 | 0.235 | 0.0001 |
| 14_15-DIHETrE | vic-Diol | SEH | nM | 0.0004 | 0.305 | 0.833 | 0.0017 |
| 11_12-DiHETrE | vic-Diol | SEH | nM | 0.0092 | 0.23 | 0.757 | 0.0538 |
| 8_9-DiHETrE | vic-Diol | SEH | nM | 0.205 | 0.762 | 0.97 | 0.87 |
| 5_6-DiHETrE | vic-Diol | SEH | nM | 0.47 | 0.507 | 0.653 | 0.0223 |
| 17_18-DiHETE | vic-Diol | SEH | nM | 0.0001 | 0.118 | 0.632 | 0.0001 |
| 19_20-DiHDoPE | vic-Diol | SEH | nM | 0.802 | 0.0983 | 0.938 | 0.623 |
| 13-HODE | R—OH | LOX | nM | 0.0793 | 0.305 | 0.96 | 0.76 |
| 9-HODE | R—OH | LOX | nM | 0.563 | 0.428 | 0.815 | 0.128 |
| 13-HOTE | R—OH | LOX | nM | 0.686 | 0.299 | 0.907 | 0.0082 |
| 9-HOTE | R—OH | LOX | nM | 0.863 | 0.976 | 0.0958 | 0.0077 |
| 15-HETE | R—OH | LOX | nM | 0.769 | 0.316 | 0.639 | 0.241 |
| 12-HETE | R—OH | LOX | nM | 0.224 | 0.751 | 0.0638 | 0.0005 |
| 11-HETE | R—OH | LOX | nM | 0.224 | 0.619 | 0.966 | 0.0021 |
| 9-HETE | R—OH | LOX | nM | 0.168 | 0.57 | 0.464 | 0.0039 |
| 8-HETE | R—OH | LOX | nM | 0.0032 | 0.984 | 0.659 | 0.0001 |
| 5-HETE | R—OH | LOX | nM | 0.0593 | 0.648 | 0.272 | 0.0001 |
| 15-HEPE | R—OH | LOX | nM | 0.116 | 0.235 | 0.979 | 0.0059 |
| 12-HEPE | R—OH | LOX | nM | 0.0073 | 0.598 | 0.792 | 0.0001 |
| 9-HEPE | R—OH | Auto-ox | nM | 0.0105 | 0.93 | 0.174 | 0.0001 |
| 5-HEPE | R—OH | LOX | nM | 0.0028 | 0.705 | 0.754 | 0.0001 |
| 14-HDoHE | R—OH | LOX | nM | 0.0171 | 0.324 | 0.574 | 0.0002 |
| 4-HDoHE | R—OH | LOX | nM | 0.0008 | 0.402 | 0.0713 | 0.0001 |
| 13-KODE | R = 0 | ADH | nM | 0.349 | 0.614 | 0.261 | 0.0061 |
| 12(13)-EpOME | Epox | CYP | nM | 0.405 | 0.719 | 0.192 | 0.0001 |
| 9(10)-EpOME | Epox | CYP | nM | 0.026 | 0.9 | 0.452 | 0.0001 |
| 15(16)-EpODE | Epox | CYP | nM | 0.206 | 0.7 | 0.414 | 0.0001 |
| 9(10)-EpODE | Epox | CYP | nM | 0.456 | 0.283 | 0.16 | 0.63 |
| 14(15)-EpETrE | Epox | CYP | nM | 0.333 | 0.101 | 0.496 | 0.076 |
| 11(12)-EpETrE | Epox | CYP | nM | 0.3 | 0.349 | 0.259 | 0.616 |
| LA | PUFA | Diet | Rel Abs | 0.0922 | 0.273 | 0.35 | 0.723 |
| ALA | PUFA | Diet | Rel Abs | 0.102 | 0.435 | 0.703 | 0.574 |
| AA | PUFA | D5D | Rel Abs | 0.0125 | 0.328 | 0.439 | 0.723 |
| EPA | PUFA | D6D | Rel Abs | 0.324 | 0.0928 | 0.611 | 0.0668 |
| DHA | PUFA | D6D | Rel Abs | 0.419 | 0.0253 | 0.913 | 0.0334 |
| OEA | Acyl-EA | PLD | nM | 0.0004 | 0.647 | 0.0846 | 0.0001 |
| LEA | Acyl-EA | PLD | nM | 0.278 | 0.527 | 0.924 | 0.732 |
| aLEA | Acyl-EA | PLD | nM | 0.72 | 0.321 | 0.437 | 0.894 |
| DGLEA | Acyl-EA | PLD | nM | 0.002 | 0.247 | 0.919 | 0.0001 |
| AEA | Acyl-EA | PLD | nM | 0.0002 | 0.968 | 0.502 | 0.0001 |
| DEA | Acyl-EA | PLD | nM | 0.0001 | 0.791 | 0.169 | 0.0001 |
| DHEA | Acyl-EA | PLD | nM | 0.0003 | 0.124 | 0.173 | 0.0001 |
| POEA | Acyl-EA | PLD | Rel Abs | 0.445 | 0.954 | 0.265 | 0.0736 |
| EPEA | Acyl-EA | PLD | Rel Abs | 0.621 | 0.597 | 1 | 0.0257 |
| NO-Gly | Acyl-Gly | FAAH | nM | 0.215 | 0.401 | 0.764 | 0.0001 |
| NA-Gly | Acyl-Gly | FAAH | nM | 0.126 | 0.791 | 0.905 | 0.951 |
| 1-AG and 2-AG | MAG | PL | nM | 0.709 | 0.349 | 0.237 | 0.0089 |
| 1-LG and 2-LG | MAG | PL | nM | 0.209 | 0.456 | 0.104 | 0.849 |
| 1-OG and 2-OG | MAG | PL | nM | 0.0184 | 0.75 | 0.0756 | 0.789 |
| Ibuprofen | NSAID | Treatment | nM | 0.725 | 0.975 | 0.737 | 0.09 |
| Naproxen | NSAID | Treatment | nM | 0.0959 | 0.66 | 0.225 | 0.0737 |
| Acetaminophen | NSAID | Treatment | nM | 0.905 | 0.868 | 0.475 | 0.778 |

Oxylipin Ratios

| | | | | |
|---|---|---|---|---|
| 12,13- | 0.0017 | 0.595 | 0.302 | 0.0001 |
| DiHOME/EpOME | | | | |
| 9_10- | 0.475 | 0.228 | 0.777 | 0.0002 |
| DiHODE/9(10)- | | | | |
| EPODE | | | | |
| 13-KODE/13- | 0.984 | 0.333 | 0.182 | 0.0022 |
| HODE | | | | |
| 11,12/14,15- | 0.0494 | 0.552 | 0.246 | 0.0029 |
| DiHETrE | | | | |
| 14,15/11,12- | 0.0494 | 0.552 | 0.246 | 0.0029 |
| DiHETrE | | | | |
| Sum(DiHOME)/ | 0.0156 | 0.606 | 0.519 | 0.0131 |
| Sum(EpOME) | | | | |
| 9,10- | 0.0141 | 0.635 | 0.247 | 0.0567 |
| DiHOME/EpOME | | | | |
| 11(12)/14(15)- | 0.636 | 0.34 | 0.0738 | 0.108 |
| EpETrE | | | | |
| 15_16-DiHODE/ | | | | |
| 15(16)-EpODE | 0.0315 | 0.416 | 0.257 | 0.127 |
| 12-HEPE/12- | 0.0303 | 0.552 | 0.278 | 0.161 |
| HETE | | | | |
| 14_15-DiHETrE/ | 0.902 | 0.257 | 0.525 | 0.427 |
| 14(15)-EpETrE | | | | |
| Sum(HDoHEs) | 0.0032 | 0.385 | 0.212 | 0.0001 |
| 17_18_DiHETE + | 0.0001 | 0.0944 | 0.645 | 0.0001 |
| 19_20_DiHDoPe | | | | |
| Sum(DiHETrE/Ep | 0.316 | 0.387 | 0.748 | 0.102 |
| ETrE) | | | | |
| Sum(DiHODE)/ | 0.051 | 0.537 | 0.242 | 0.272 |
| Sum(EpODE) | | | | |

Bile Acids

| | | | | | | | |
|---|---|---|---|---|---|---|---|
| CA | 1°-BA | Cyp27A1; | nM | 0.0321 | 0.359 | 0.417 | 0.0406 |
| | | CYP8B1 | | | | | |
| CDCA | 1°-BA | Cyp27A1 | nM | 0.978 | 0.212 | 0.677 | 0.158 |
| UDCA | 2°-BA | Microbiome | nM | 0.625 | 0.56 | 0.245 | 0.972 |
| DCA | 2°-BA | Microbiome | nM | 0.696 | 0.0157 | 0.316 | 0.799 |
| LCA | 2°-BA | Microbiome | nM | 0.411 | 0.228 | 0.797 | 0.293 |
| w-MCA | 2°-BA | Cyp27A1 | nM | 0.836 | 0.185 | 0.0573 | 0.22 |
| b-MCA | 2°-BA | Cyp27A1 | nM | 0.248 | 0.621 | 0.43 | 0.717 |
| TCA | 1°-BA | BAT | nM | 0.39 | 0.594 | 0.0999 | 0.0784 |
| | Conj | | | | | | |
| TCDCA | 1°-BA | BAT | nM | 0.726 | 0.42 | 0.149 | 0.0007 |
| | Conj | | | | | | |
| TUDCA | 2°-BA | BAT | nM | 0.0275 | 0.777 | 0.887 | 0.0001 |
| | Conj | | | | | | |
| TDCA | 2°-BA | BAT | nM | 0.23 | 0.989 | 0.118 | 0.0019 |
| | Conj | | | | | | |
| TLCA | 2°-BA | BAT | nM | 0.597 | 0.947 | 0.693 | 0.259 |
| | Conj | | | | | | |
| GCA | 1°-BA | BAT | nM | 0.982 | 0.74 | 0.0467 | 0.0035 |
| | Conj | | | | | | |
| GCDCA | 1°-BA | BAT | nM | 0.354 | 0.584 | 0.205 | 0.0001 |
| | Conj | | | | | | |
| GUDCA | 2°-BA | BAT | nM | 0.453 | 0.607 | 0.193 | 0.0004 |
| | Conj | | | | | | |
| GDCA | 2°-BA | BAT | nM | 0.0653 | 0.132 | 0.146 | 0.0001 |
| | Conj | | | | | | |
| GHDCA | 2°-BA | BAT | nM | 0.228 | 0.335 | 0.936 | 0.571 |
| | Conj | | | | | | |
| GLCA | 2°-BA | BAT | nM | 0.185 | 0.281 | 0.549 | 0.0003 |
| | Conj | | | | | | |
| T-a-MCA | 2°-BA | BAT | nM | 0.818 | 0.778 | 0.154 | 0.135 |
| | Conj | | | | | | |

Bile Acid Ratios

| | | | | |
|---|---|---|---|---|
| CA/CDCA | 0.0008 | 0.788 | 0.179 | 0.0001 |
| GDCA/DCA | 0.0031 | 0.43 | 0.405 | 0.0001 |
| TDCA/CA | 0.0056 | 0.244 | 0.82 | 0.0001 |
| GCA/CA | 0.0104 | 0.314 | 0.838 | 0.0001 |
| GDCA/CA | 0.0007 | 0.664 | 0.946 | 0.0001 |
| TDCA/TLCA | 0.0114 | 0.829 | 0.0424 | 0.0003 |
| (GDCA + GLCA)/ | 0.01 | 0.0131 | 0.707 | 0.0003 |
| (TDCA + TLCA) | | | | |
| TCA/CA | 0.076 | 0.223 | 0.923 | 0.0004 |
| TDCA/DCA | 0.1 | 0.0549 | 0.448 | 0.0019 |
| DCA/CA | 0.0459 | 0.875 | 0.944 | 0.021 |
| GUDCA/UDCA | 0.7 | 0.769 | 0.952 | 0.0584 |
| GLCA/CDCA | 0.273 | 0.975 | 0.654 | 0.0617 |
| GCDCA/CDCA | 0.491 | 0.519 | 0.584 | 0.0688 |
| GCA/GDCA | 0.0679 | 0.0556 | 0.493 | 0.204 |
| w-MCA/UDCA | 0.662 | 0.549 | 0.544 | 0.23 |
| GCDCA/GLCA | 0.256 | 0.429 | 0.568 | 0.445 |
| w-MCA/T-a-MCA | 0.431 | 0.353 | 0.476 | 0.529 |
| GDCA/GLCA | 0.988 | 0.61 | 0.224 | 0.676 |
| TLCA/CDCA | 0.651 | 0.674 | 0.821 | 0.734 |
| T-a-MCA/CDCA | 0.913 | 0.35 | 0.363 | 0.929 |
| (GCA + TCA)/CA | 0.0148 | 0.175 | 0.839 | 0.0001 |
| (GLCA + TLCA)/LCA | 0.719 | 0.314 | 0.473 | 0.0657 |
| (TCA + GCA + | 0.809 | 0.456 | 0.45 | 0.705 |
| TDCA + GDCA)/ | | | | |
| (GUDCA + TUDCA + | | | | |
| GLCA + TLCA + | | | | |
| TCDCA + GCDCA) | | | | |
| (TCDCA + GCDCA)/ | 0.479 | 0.45 | 0.549 | 0.0706 |
| CDCA | | | | |
| (TUDAC + GUDCA)/ | 0.746 | 0.786 | 0.942 | 0.0635 |
| UDCA | | | | |
| DCA/(LCA + UDCA) | 0.524 | 0.396 | 0.765 | 0.522 |
| LCA/CDCA | 0.451 | 0.585 | 0.483 | 0.859 |
| (TDCA + GDCA)/DCA | 0.0034 | 0.207 | 0.379 | 0.0001 |
| UDCA/CDCA | 0.691 | 0.49 | 0.847 | 0.329 |
| GLCA/LCA | 0.721 | 0.521 | 0.601 | 0.0867 |
| TCDCA/CDCA | 0.644 | 0.122 | 0.535 | 0.179 |
| TLCA/LCA | 0.961 | 0.0545 | 0.71 | 0.286 |
| TUDCA/UDCA | 0.746 | 0.949 | 0.997 | 0.234 |

Steroids

| | | | | | | | |
|---|---|---|---|---|---|---|---|
| CRTL | Steroid | 11-beta-HSD; | nM | 0.597 | 0.719 | 0.676 | 0.439 |
| | | Cyp11B1 | | | | | |
| CRTN | Steroid | 11-beta-HSD | nM | 0.188 | 0.426 | 0.294 | 0.13 |
| CRCTN | Steroid | Cyp11B1 | nM | 0.386 | 0.686 | 0.293 | 0.669 |
| 11-Deoxy-CTRL | Steroid | CYP8B1 | nM | 0.952 | 0.298 | 0.671 | 0.572 |
| DHEAS | Steroid | | nM | 0.0057 | 0.595 | 0.705 | 0.0002 |
| TEST | Steroid | 3-beta-HSD | nM | 0.945 | 0.0016 | 0.209 | 0.0239 |
| 17OH-PROG | Steroid | 3-beta-HSD | nM | 0.0517 | 0.338 | 0.985 | 0.0006 |
| Progesterone | Steroid | Sex | nM | 0.0001 | 0.661 | 0.0876 | 0.0001 |
| | | Hormones | | | | | |

Steroid Ratios

| | | | | |
|---|---|---|---|---|
| Tes/Prog | 0.0021 | 0.0152 | 0.767 | 0.18 |

| | Mean [95% CI] Using subjects | | | |
|---|---|---|---|---|
| Metabolite | with p of fasted >60% | | | |
| information | Control mean | | AD mean | |
| Metabolite name | [95% CI] | | [95% CI] | |
| Oxylipins, endocannabinoids, PUFAS, and NSAIDS | | | | |
| TXB2 | 0.327 | [0.262-0.408] | 0.234 | [0.178-0.307] |
| PGE2 | 0.209 | [0.182-0.238] | 0.16 | [0.13-0.197] |
| PGD2 | 0.355 | [0.286-0.441] | 0.225 | [0.142-0.355] |
| PGF2a | 0.65 | [0.525-0.805] | 0.387 | [0.284-0.527] |
| F2-IsoP | 8.22 | [7.15-9.45] | 6.36 | [5.33-7.59] |
| 5_15-DiHETE | 0.0998 | [0.0777-0.128] | 0.106 | [0.0717-0.158] |
| 9_12_13-TriHOME | 4.38 | [3.75-5.1] | 2.65 | [2.2-3.18] |
| 9_10-e-DiHO | 3.97 | [3.56-4.42] | 3.05 | [2.68-3.46] |
| 12_13-DiHOME | 4.71 | [4.26-5.21] | 3.86 | [3.38-4.41] |
| 9_10-DiHOME | 3.16 | [2.89-3.45] | 3.25 | [2.94-3.59] |
| 15_16-DiHODE | 11.6 | [10.5-12.8] | 10.9 | [9.47-12.5] |
| 12_13-DiHODE | 0.218 | [0.176-0.27] | 0.202 | [0.151-0.27] |
| 9_10-DiHODE | 0.192 | [0.162-0.228] | 0.184 | [0.141-0.24] |
| 14_15-DiHETrE | 0.833 | [0.784-0.886] | 1 | [0.912-1.1] |
| 11_12-DiHETrE | 0.709 | [0.67-0.75] | 0.81 | [0.735-0.894] |
| 8_9-DiHETrE | 0.266 | [0.203-0.347] | 0.339 | [0.24-0.478] |
| 5_6-DiHETrE | 0.565 | [0.513-0.622] | 0.51 | [0.449-0.581] |
| 17_18-DiHETE | 1.73 | [1.32-2.27] | 5.23 | [4.32-6.34] |
| 19_20-DiHDoPE | 2 | [1.83-2.18] | 2.03 | [1.78-2.33] |
| 13-HODE | 16.8 | [15.4-18.3] | 14.3 | [12.7-16.1] |
| 9-HODE | 11.9 | [10.9-13] | 10.9 | [9.66-12.2] |
| 13-HOTE | 0.709 | [0.601-0.836] | 0.624 | [0.483-0.806] |
| 9-HOTE | 0.479 | [0.424-0.542] | 0.446 | [0.39-0.511] |
| 15-HETE | 3.17 | [2.82-3.55] | 3.13 | [2.81-3.49] |
| 12-HETE | 3.35 | [2.95-3.81] | 2.81 | [2.46-3.21] |
| 11-HETE | 2.01 | [1.75-2.3] | 1.61 | [1.4-1.86] |
| 9-HETE | 1.73 | [1.47-2.03] | 1.32 | [1.12-1.57] |
| 8-HETE | 3.64 | [3.18-4.17] | 2.51 | [2.16-2.93] |
| 5-HETE | 5.02 | [4.43-5.68] | 3.79 | [3.35-4.29] |
| 15-HEPE | 0.345 | [0.282-0.422] | 0.294 | [0.24-0.359] |
| 12-HEPE | 0.459 | [0.39-0.539] | 0.313 | [0.253-0.387] |
| 9-HEPE | 0.319 | [0.256-0.397] | 0.158 | [0.103-0.241] |
| 5-HEPE | 0.908 | [0.771-1.07] | 0.572 | [0.463-0.706] |
| 14-HDoHE | 2.4 | [2-2.88] | 1.61 | [1.17-2.21] |
| 4-HDoHE | 1.03 | [0.886-1.21] | 0.651 | [0.545-0.777] |
| 13-KODE | 1.74 | [1.43-2.12] | 1.46 | [1.18-1.81] |
| 12(13)-EpOME | 2.28 | [2.03-2.56] | 2.41 | [2.07-2.8] |
| 9(10)-EpOME | 0.29 | [0.244-0.346] | 0.369 | [0.3-0.453] |
| 15(16)-EpODE | 2.14 | [1.9-2.4] | 2.42 | [2.06-2.84] |
| 9(10)-EpODE | 0.174 | [0.147-0.206] | 0.146 | [0.11-0.193] |
| 14(15)-EpETrE | 0.0851 | [0.0634-0.114] | 0.1 | [0.074-0.136] |
| 11(12)-EpETrE | 0.0574 | [0.0439-0.0751] | 0.0705 | [0.0507-0.0979] |
| LA | 0.154 | [0.143-0.166] | 0.158 | [0.138-0.18] |
| ALA | 0.146 | [0.131-0.162] | 0.153 | [0.128-0.184] |
| AA | 0.138 | [0.126-0.151] | 0.155 | [0.139-0.172] |
| EPA | 0.12 | [0.105-0.138] | 0.13 | [0.108-0.156] |
| DHA | 0.147 | [0.133-0.162] | 0.149 | [0.128-0.174] |
| OEA | 7.31 | [6.63-8.06] | 5.23 | [4.79-5.7] |
| LEA | 4.26 | [3.9-4.65] | 4.5 | [4.13-4.91] |
| aLEA | 0.179 | [0.161-0.199] | 0.184 | [0.164-0.207] |
| DGLEA | 0.245 | [0.216-0.276] | 0.175 | [0.151-0.201] |
| AEA | 2.2 | [1.97-2.46] | 1.49 | [1.36-1.64] |
| DEA | 0.664 | [0.571-0.771] | 0.378 | [0.325-0.44] |
| DHEA | 1.67 | [1.5-1.86] | 1.18 | [1.04-1.34] |
| POEA | 0.127 | [0.111-0.145] | 0.119 | [0.0985-0.144] |
| EPEA | 0.102 | [0.0878-0.119] | 0.0873 | [0.0704-0.108] |
| NO-Gly | 5.21 | [4.8-5.66] | 4.7 | [4.29-5.16] |
| NA-Gly | 0.609 | [0.533-0.695] | 0.72 | [0.621-0.835] |
| 1-AG and 2-AG | 25.3 | [22.8-28] | 23.8 | [21.2-26.8] |
| 1-LG and 2-LG | 260 | [229-295] | 222 | [197-250] |
| 1-OG and 2-OG | 409 | [358-467] | 318 | [284-355] |
| Ibuprofen | 64.2 | [38.6-107] | 41 | [21.4-78.4] |
| Naproxen | 10.3 | [5.31-19.9] | 31.4 | [7.51-132] |
| Acetaminophen | 5.14 | [3.09-8.54] | 5.06 | [2.61-9.82] |
| Oxylipin Ratios | | | | |
| 12,13- | 2.07 | [1.93-2.22] | 1.6 | [1.43-1.79] |
| DiHOME/EpOME | | | | |
| 9_10- | 1.09 | [0.87-1.36] | 1.26 | [0.861-1.84] |
| DiHODE/9(10)- | | | | |
| EpODE | | | | |
| 13-KODE/13- | 0.103 | [0.0867-0.123] | 0.104 | [0.0854-0.126] |
| HODE | | | | |
| 11,12/14,15- | 0.851 | [0.825-0.878] | 0.809 | [0.771-0.849] |
| DiHETrE | | | | |
| 14,15/11,12- | 1.18 | [1.14-1.21] | 1.24 | [1.18-1.3] |
| DiHETrE | | | | |
| Sum(DiHOME)/Su | 3.02 | [2.8-3.26] | 2.5 | [2.24-2.78] |
| m(EpOME) | | | | |
| 9,10- | 11.7 | [10-13.7] | 8.97 | [7.43-10.8] |
| DiHOME/EpOME | | | | |
| 11(12)/14(15)- | 0.781 | [0.499-1.22] | 0.688 | [0.422-1.12] |
| EpETrE | | | | |
| 15_16-DiHODE/ | | | | |
| 15(16)-EpODE | 5.43 | [5-5.89] | 4.49 | [3.98-5.05] |
| 12-HEPE/12- | 0.137 | [0.121-0.155] | 0.11 | [0.0896-0.135] |
| HETE | | | | |
| 14_15-DiHETrE/ | 10.1 | [7.66-13.4] | 10.1 | [7.3-14.1] |
| 14(15)-EpETrE | | | | |
| Sum(HDoHEs) | 3.82 | [3.37-4.34] | 2.66 | [2.19-3.24] |
| 17_18_DiHETE + | 4.45 | [3.88-5.11] | 7.44 | [6.3-8.78] |
| 19_20_DiHDoPe | | | | |
| Sum(DiHETrE/Ep | 20.1 | [16.4-24.6] | 17.1 | [13.1-22.3] |
| ETrE) | | | | |
| Sum(DiHODE)/ | 5.12 | [4.74-5.53] | 4.34 | [3.86-4.88] |
| Sum(EpODE) | | | | |
| Bile Acids | | | | |
| CA | 27.4 | [20.8-36.2] | 19.3 | [13.6-27.4] |
| CDCA | 38.7 | [29.2-51.5] | 46.7 | [32.6-66.9] |
| UDCA | 68.3 | [46.5-100] | 90.1 | [61.4-132] |
| DCA | 219 | [184-261] | 177 | [120-259] |
| LCA | 32.2 | [25.4-40.8] | 29.3 | [23-37.4] |
| w-MCA | 7.34 | [4.85-11.1] | 7.63 | [5.46-10.7] |
| b-MCA | 1.42 | [0.924-2.2] | 1.96 | [1.32-2.91] |
| TCA | 13.9 | [11-17.5] | 11.7 | [8.1-16.9] |
| TCDCA | 28.4 | [22.6-35.8] | 30.3 | [21.6-42.7] |
| TUDCA | 1.07 | [0.78-1.48] | 1.92 | [1.53-2.41] |
| TDCA | 12.8 | [9.84-16.8] | 13.7 | [8.95-21] |
| TLCA | 9.65 | [7.95-11.7] | 7.56 | [5.18-11.1] |
| GCA | 68.9 | [56-84.8] | 68.6 | [51.6-91.1] |
| GCDCA | 316 | [260-384] | 367 | [270-499] |
| GUDCA | 41 | [32.9-51.2] | 46.3 | [32.8-65.4] |
| GDCA | 157 | [127-195] | 186 | [125-277] |
| GHDCA | 0.891 | [0.688-1.15] | 0.72 | [0.53-0.979] |
| GLCA | 15.4 | [12.5-19] | 21.8 | [14.8-32.1] |
| T-a-MCA | 2.89 | [2.4-3.49] | 2.62 | [1.9-3.63] |
| Bile Acid Ratios | | | | |
| CA/CDCA | 0.707 | [0.571-0.876] | 0.363 | [0.271-0.487] |
| GDCA/DCA | 0.719 | [0.604-0.856] | 1.05 | [0.836-1.33] |
| TDCA/CA | 0.469 | [0.309-0.711] | 0.906 | [0.517-1.59] |
| GCA/CA | 2.52 | [1.86-3.39] | 4.13 | [2.84-6.01] |
| GDCA/CA | 5.76 | [4.01-8.27] | 12.5 | [7.71-20.2] |
| TDCA/TLCA | 1.33 | [1.1-1.62] | 1.81 | [1.49-2.21] |
| (GDCA + GLCA)/ | 7.04 | [6.18-8.02] | 9.61 | [7.9-11.7] |
| (TDCA + TLCA) | | | | |
| TCA/CA | 0.506 | [0.358-0.716] | 0.711 | [0.434-1.16] |
| TDCA/DCA | 0.0605 | [0.0467-0.0783] | 0.0777 | [0.0556-0.109] |
| DCA/CA | 8.01 | [5.8-11.1] | 12 | [7.89-18.1] |
| GUDCA/UDCA | 0.546 | [0.341-0.876] | 0.515 | [0.334-0.793] |
| GLCA/CDCA | 0.397 | [0.278-0.567] | 0.487 | [0.291-0.817] |
| GCDCA/CDCA | 8.15 | [6.21-10.7] | 8.36 | [5.68-12.3] |
| GCA/GDCA | 0.431 | [0.357-0.519] | 0.368 | [0.277-0.49] |
| w-MCA/UDCA | 0.108 | [0.0659-0.175] | 0.0845 | [0.054-0.132] |
| GCDCA/GLCA | 20.5 | [16.2-26] | 16.8 | [11.9-23.7] |
| w-MCA/T-a-MCA | 3.16 | [1.86-5.38] | 2.86 | [1.82-4.5] |
| GDCA/GLCA | 9.92 | [8.33-11.8] | 8.53 | [6.67-10.9] |
| TLCA/CDCA | 0.249 | [0.17-0.364] | 0.169 | [0.0992-0.287] |
| T-a-MCA/CDCA | 0.0747 | [0.0524-0.106] | 0.057 | [0.0351-0.0926] |
| (GCA + TCA)/CA | 3.12 | [2.3-4.23] | 5.04 | [3.4-7.47] |
| (GLCA + TLCA)/LCA | 1.08 | [0.815-1.44] | 1.11 | [0.831-1.49] |
| (TCA + GCA + | 0.637 | [0.561-0.724] | 0.606 | [0.511-0.72] |
| TDCA + GDCA)/ | | | | |
| (GUDCA + TUDCA + | | | | |
| GLCA + TLCA + | | | | |
| TCDCA + GCDCA) | | | | |
| (TCDCA + GCDCA)/ | 9.06 | [6.88-11.9] | 9.31 | [6.3-13.7] |
| CDCA | | | | |
| (TUDAC + GUDCA)/ | 0.589 | [0.369-0.938] | 0.54 | [0.351-0.832] |
| UDCA | | | | |
| DCA/(LCA + UDCA) | 1.9 | [1.42-2.55] | 1.43 | [0.971-2.1] |
| LCA/CDCA | 0.66 | [0.419-1.04] | 0.589 | [0.42-0.825] |
| (TDCA + GDCA)/DCA | 0.794 | [0.664-0.949] | 1.17 | [0.919-1.48] |
| UDCA/CDCA | 1.39 | [0.815-2.36] | 1.94 | [1.23-3.07] |
| GLCA/LCA | 0.711 | [0.53-0.955] | 0.749 | [0.552-1.02] |
| TCDCA/CDCA | 0.734 | [0.53-1.01] | 0.687 | [0.433-1.09] |
| TLCA/LCA | 0.282 | [0.2-0.396] | 0.252 | [0.182-0.347] |
| TUDCA/UDCA | 2.83 | [1.93-4.17] | 2.04 | [1.33-3.13] |
| Steroids | | | | |
| CRTL | 252 | [231-274] | 207 | [158-272] |
| CRTN | 46.6 | [43.6-49.9] | 37.4 | [29.5-47.3] |
| CRCTN | 4.46 | [3.84-5.18] | 4.9 | [3.82-6.27] |
| 11-Deoxy-CTRL | 0.775 | [0.701-0.857] | 0.768 | [0.601-0.98] |
| DHEAS | 2000 | [1410-2840] | 1070 | [726-1570] |
| TEST | 1.41 | [1.1-1.81] | 2.5 | [1.63-3.85] |
| 17OH-PROG | 1.04 | [0.872-1.24] | 0.951 | [0.761-1.19] |
| Progesterone | 0.617 | [0.542-0.701] | 0.37 | [0.317-0.432] |
| Steroid Ratios | | | | |
| Tes/Prog | 2.29 | [1.75-3.01] | 6.76 | [4.34-10.5] |

TABLE 9
The mean values and t-test and two-way ANOVA interaction
p-values for all detected metabolites in CSF.

| | | | | p-values | | |
|---|---|---|---|---|---|---|
| | | | | | p-value | p-value |
| Metabolite information | | | | p-value | Gender × | Race × |
| | Chemical | | | AD vs | Disease | Disease |
| Metabolite name | class | Enzyme | Units | Control | interaction | interaction |

Oxylipins, endocannabinoids, PUFAS, and NSAIDs

| | | | | | | |
|---|---|---|---|---|---|---|
| PGF2a | PGs | COX2 | nM | 0.0028 | 0.0964 | 0.537 |
| F2-IsoP | PGs | Auto-ox | nM | 0.105 | 0.57 | 0.0295 |
| 12_13-DiHOME | vic-Diol | SEH | nM | 0.0008 | 0.435 | 0.407 |
| 9_10-DiHOME | vic-Diol | SEH | nM | 0.0003 | 0.476 | 0.161 |
| 15_16-DiHODE | vic-Diol | SEH | nM | 0.12 | 0.8 | 0.922 |
| 14_15-DiHETrE | vic-Diol | SEH | nM | 0.501 | 0.744 | 0.674 |
| 11_12-DiHETrE | vic-Diol | SEH | nM | 0.0315 | 0.566 | 0.477 |
| 17_18-DiHETE | vic-Diol | SEH | nM | 0.34 | 0.932 | 0.942 |
| 14_15-DiHETE | vic-Diol | SEH | nM | 0.0022 | 0.939 | 0.661 |
| 19_20-DiHDoPE | vic-Diol | SEH | nM | 0.588 | 0.128 | 0.346 |
| 13-HODE | R—OH | LOX | nM | 0.67 | 0.285 | 0.244 |
| 9-HODE | R—OH | LOX | nM | 0.517 | 0.346 | 0.119 |
| 13-HOTE | R—OH | LOX | nM | 0.469 | 0.0108 | 0.583 |
| 9-HOTE | R—OH | LOX | nM | 0.966 | 0.298 | 0.591 |
| 12(13)-EpOME | Epox | CYP | nM | 0.0001 | 0.0414 | 0.522 |
| 9(10)-EpOME | Epox | CYP | nM | 0.0001 | 0.0303 | 0.584 |
| 20-HETE | R-OH | CYP | nM | 0.0791 | 0.539 | 0.471 |
| LA | PUFA | Diet | Rel Abs | 0.0001 | 0.377 | 0.581 |
| ALA | PUFA | Diet | Rel Abs | 0.0146 | 0.104 | 0.739 |
| AA | PUFA | D5D | Rel Abs | 0.0001 | 0.508 | 0.948 |
| EPA | PUFA | D6D | Rel Abs | 0.0001 | 0.648 | 0.444 |
| DHA | PUFA | D6D | Rel Abs | 0.0118 | 0.322 | 0.663 |
| OEA | Acyl-EA | PLD | nM | 0.0001 | 0.489 | 0.0418 |
| LEA | Acyl-EA | PLD | nM | 0.0934 | 0.0299 | 0.655 |
| DHEA | Acyl-EA | PLD | nM | 0.813 | 0.469 | 0.758 |

Oxylipin Ratios

| | | | |
|---|---|---|---|
| 9(10)/12(13)-EpOME | 0.0001 | 0.401 | 0.98 |
| 11,12/14,15-DiHETrE | 0.0009 | 0.56 | |
| 9,10/12,13-DiHOME | 0.558 | 0.593 | |
| 14,15/17,18-DiHETE | 0.171 | 0.748 | |
| 12,13-DiHOME/EpOME | 0.0615 | 0.875 | 0.506 |
| 14,15/11,12-DiHETrE | 0.0008 | 0.544 | 0.46 |
| 9,10-DiHOME/EpOME | 0.902 | 0.656 | 0.409 |
| EPA + DHA diols | 0.178 | 0.82 | 0.801 |
| Sum(DiHOME)/ | 0.248 | 0.786 | 0.474 |
| Sum(EpOME) | | | |
| Sum(DiHOMEs) | 0.0007 | 0.356 | 0.267 |
| Sum(EpOMEs) | 0.0001 | 0.0688 | 0.585 |
| Sum(LA Cyp + sEH) | 0.0001 | 0.0231 | 0.407 |

Bile Acids

| | | | | | | |
|---|---|---|---|---|---|---|
| CDCA | 1°-BA | Cyp27A1 | nM | 0.756 | 0.234 | 0.152 |
| UDCA | 2°-BA | Microbiome | nM | 0.425 | 0.0021 | 0.678 |
| DCA | 2°-BA | Microbiome | nM | 0.601 | 0.0787 | 0.216 |
| a-MCA | 2°-BA | Cyp27A1 | nM | 0.319 | 0.751 | 0.416 |
| w-MCA | 2°-BA | Cyp27A1 | nM | 0.267 | 0.36 | 0.0969 |
| TCA | 1°-BA Conj | BAT | nM | 0.291 | 0.164 | 0.182 |
| TCDCA | 1°-BA Conj | BAT | nM | 0.0552 | 0.75 | 0.303 |
| TUDCA | 2°-BA Conj | BAT | nM | 0.0993 | 0.46 | 0.349 |
| TDCA | 2°-BA Conj | BAT | nM | 0.878 | 0.234 | 0.0575 |
| GCA | 1°-BA Conj | BAT | nM | 0.98 | 0.277 | 0.0429 |
| GCDCA | 1°-BA Conj | BAT | nM | 0.267 | 0.79 | 0.0913 |
| GUDCA | 2°-BA Conj | BAT | nM | 0.548 | 0.872 | 0.109 |
| GDCA | 2°-BA Conj | BAT | nM | 0.345 | 0.17 | 0.0303 |
| GLCA | 2°-BA Conj | BAT | nM | 0.0185 | 0.299 | 0.491 |
| T-a-MCA | 2°-BA Conj | BAT | nM | 0.0262 | 0.433 | 0.463 |

Bile Acid Ratios

| | | | |
|---|---|---|---|
| (GDCA + GLCA)/ | 0.0495 | 0.578 | 0.549 |
| (TDCA) | | | |
| (GDCA + TDCA)/ | 0.154 | 0.0513 | 0.0332 |
| (TLCA + GLCA) | | | |
| (GDCA + TDCA)/ | 0.981 | 0.538 | 0.991 |
| (TUDCA + GUDCA) | | | |
| (TCDCA + GCDCA)/ | 0.241 | 0.0782 | 0.841 |
| CDCA | | | |
| (TDCA + GDCA)/ | 0.623 | 0.274 | 0.327 |
| DCA | | | |
| (TDCA + TCDCA)/ | 0.161 | 0.576 | 0.975 |
| (GDCA + GCDCA) | | | |
| (TUDAC + GUDCA)/ | 0.881 | 0.0525 | 0.347 |
| UDCA | | | |
| DCA/(LCA + UDCA) | 0.587 | 0.283 | 0.632 |
| GCA/GCDCA | 0.161 | 0.143 | 0.291 |
| GCA/GDCA | 0.666 | 0.671 | 0.966 |
| GCDCA/CDCA | 0.268 | 0.0832 | 0.832 |
| GCDCA/GDCA | 0.164 | 0.21 | 0.579 |
| GCDCA/GLCA | 0.0029 | 0.533 | 0.0727 |
| GDCA/DCA | 0.647 | 0.212 | 0.249 |
| GDCA/GLCA | 0.161 | 0.0588 | 0.0376 |
| GLCA/CDCA | 0.129 | 0.0427 | 0.0693 |
| GUDCA/UDCA | 0.86 | 0.0606 | 0.34 |
| T-a-MCA/CDCA | 0.18 | 0.122 | 0.684 |
| TCDCA/CDCA | 0.0996 | 0.104 | 0.759 |
| TCDCA/GCDCA | 0.125 | 0.512 | 0.714 |
| TDCA/DCA | 0.708 | 0.369 | 0.458 |
| TDCA/GDCA | 0.204 | 0.589 | 0.822 |
| TUDCA/UDCA | 0.593 | 0.0018 | 0.717 |
| UDCA/CDCA | 0.423 | 0.019 | 0.374 |
| w-MCA/T-a-MCA | 0.863 | 0.184 | 0.379 |
| w-MCA/UDCA | 0.705 | 0.265 | 0.2 |

Steroids

| | | | | | | |
|---|---|---|---|---|---|---|
| CRTL | Steroid | 11-beta-HSD; | nM | 0.466 | 0.798 | 0.975 |
| | | Cyp11B1 | | | | |
| CRTN | Steroid | 11-beta-HSD | nM | 0.0048 | 0.25 | 0.378 |
| CRCTN | Steroid | Cyp11B1 | nM | 0.503 | 0.226 | 0.995 |
| 11-Deoxy-CTRL | Steroid | CYP8B1 | nM | 0.0803 | 0.0648 | 0.132 |
| TEST | Steroid | 3-beta-HSD | nM | 0.49 | 0.245 | 0.827 |
| 17OH-PROG | Steroid | 3-beta-HSD | nM | 0.109 | 0.408 | 0.525 |

| | Mean [95% CI] Uning subjects | | | |
|---|---|---|---|---|
| Metabolite | with p of fasted >60% | | | |
| information | Control mean | | AD mean | |
| Metabolite name | [95% CI] | | [95% CI] | |
| Oxylipins, endocannabinoids, PUFAS, and NSAIDs | | | | |
| PGF2a | 0.0356 | [0.0313-0.0404] | 0.0305 | [0.0278-0.0335] |
| F2-IsoP | 0.269 | [0.256-0.284] | 0.295 | [0.281-0.309] |
| 12_13-DiHOME | 0.0275 | [0.0245-0.0308] | 0.0356 | [0.0321-0.0394] |
| 9_10-DiHOME | 0.016 | [0.014-0.0182] | 0.0218 | [0.0196-0.0243] |
| 15_16-DiHODE | 0.0765 | [0.0667-0.0877] | 0.0735 | [0.0662-0.0816] |
| 14_15-DiHETrE | 0.0461 | [0.0433-0.0491] | 0.0473 | [0.0445-0.0503] |
| 11_12-DiHETrE | 0.0179 | [0.0166-0.0193] | 0.017 | [0.0158-0.0184] |
| 17_18-DiHETE | 0.0905 | [0.0784-0.104] | 0.0896 | [0.0776-0.103] |
| 14_15-DiHETE | 0.0294 | [0.0254-0.0339] | 0.023 | [0.0195-0.0272] |
| 19_20-DiHDoPE | 0.0155 | [0.014-0.0171] | 0.0176 | [0.0162-0.0192] |
| 13-HODE | 1.61 | [1.56-1.66] | 1.6 | [1.56-1.65] |
| 9-HODE | 0.556 | [0.535-0.577] | 0.542 | [0.525-0.56] |
| 13-HOTE | 0.0497 | [0.0468-0.0527] | 0.052 | [0.0482-0.0561] |
| 9-HOTE | 0.0121 | [0.0113-0.013] | 0.0123 | [0.0114-0.0132] |
| 12(13)-EpOME | 0.215 | [0.196-0.236] | 0.335 | [0.304-0.369] |
| 9(10)-EpOME | 0.219 | [0.202-0.237] | 0.31 | [0.277-0.348] |
| 20-HETE | 0.138 | [0.126-0.152] | 0.126 | [0.118-0.135] |
| LA | 0.296 | [0.278-0.315] | 0.236 | [0.224-0.249] |
| ALA | 0.293 | [0.276-0.31] | 0.265 | [0.253-0.279] |
| AA | 0.308 | [0.287-0.329] | 0.247 | [0.232-0.262] |
| EPA | 0.28 | [0.249-0.314] | 0.203 | [0.184-0.224] |
| DHA | 0.284 | [0.261-0.308] | 0.257 | [0.236-0.279] |
| OEA | 0.126 | [0.116-0.137] | 0.0866 | [0.0813-0.0922] |
| LEA | 0.24 | [0.23-0.251] | 0.256 | [0.246-0.267] |
| DHEA | 0.0136 | [0.0119-0.0155] | 0.016 | [0.0147-0.0173] |
| Oxylipin Ratios | | | | |
| 9(10)/12(13)-EpOME | 1.02 | [0.972-1.06] | 0.928 | [0.9-0.956] |
| 11,12/14,15-DiHETrE | 0.389 | [0.377-0.401] | 0.36 | [0.347-0.373] |
| 9,10/12,13-DiHOME | 0.582 | [0.532-0.638] | 0.613 | [0.576-0.653] |
| 14,15/17,18-DiHETE | 0.326 | [0.272-0.391] | 0.259 | [0.214-0.314] |
| 12,13-DiHOME/EpOME | 0.128 | [0.112-0.146] | 0.106 | [0.0939-0.12] |
| 14,15/11,12-DiHETrE | 2.57 | [2.5-2.65] | 2.78 | [2.68-2.88] |
| 9,10-DiHOME/EpOME | 0.0732 | [0.0646-0.0831] | 0.0703 | [0.0611-0.0809] |
| EPA + DHA diols | 0.147 | [0.133-0.161] | 0.145 | [0.132-0.159] |
| Sum(DiHOME)/ | 0.103 | [0.0917-0.115] | 0.0902 | [0.0795-0.102] |
| Sum(EpOME) | | | | |
| Sum(DiHOMEs) | 0.0449 | [0.0402-0.0501] | 0.0584 | [0.0529-0.0644] |
| Sum(EpOMEs) | 0.437 | [0.403-0.474] | 0.648 | [0.584-0.718] |
| Sum(LA Cyp + sEH) | 0.492 | [0.455-0.531] | 0.723 | [0.658-0.796] |
| Bile Acids | | | | |
| CDCA | 1.06 | [0.887-1.27] | 1.23 | [1.06-1.42] |
| UDCA | 0.699 | [0.58-0.842] | 0.886 | [0.719-1.09] |
| DCA | 1.81 | [1.57-2.1] | 1.99 | [1.67-2.38] |
| a-MCA | 0.0398 | [0.0317-0.0499] | 0.033 | [0.0263-0.0414] |
| w-MCA | 0.0826 | [0.0652-0.105] | 0.106 | [0.0819-0.136] |
| TCA | 0.405 | [0.336-0.487] | 0.503 | [0.418-0.605] |
| TCDCA | 0.0797 | [0.0675-0.094] | 0.0704 | [0.0603-0.0822] |
| TUDCA | 0.00272 | [0.0022-0.00336] | 0.00351 | [0.00293-0.0042] |
| TDCA | 0.0463 | [0.0397-0.054] | 0.0459 | [0.0381-0.0553] |
| GCA | 0.907 | [0.79-1.04] | 0.997 | [0.862-1.15] |
| GCDCA | 0.79 | [0.691-0.902] | 0.808 | [0.723-0.903] |
| GUDCA | 0.0879 | [0.0734-0.105] | 0.0992 | [0.0833-0.118] |
| GDCA | 0.367 | [0.319-0.421] | 0.414 | [0.352-0.488] |
| GLCA | 0.0278 | [0.024-0.0321] | 0.0368 | [0.0319-0.0425] |
| T-a-MCA | 0.0221 | [0.0186-0.0264] | 0.0303 | [0.0256-0.0358] |
| Bile Acid Ratios | | | | |
| (GDCA + GLCA)/ | 8.88 | [8.07-9.78] | 10.5 | [9.26-11.9] |
| (TDCA) | | | | |
| (GDCA + TDCA)/ | 15.2 | [12.8-18] | 13 | [10.8-15.5] |
| (TLCA + GLCA) | | | | |
| (GDCA + TDCA)/ | 4.58 | [3.73-5.62] | 4.55 | [3.66-5.66] |
| (TUDCA + GUDCA) | | | | |
| (TCDCA + GCDCA)/ | 0.831 | [0.707-0.977] | 0.727 | [0.632-0.837] |
| CDCA | | | | |
| (TDCA + GDCA)/ | 0.233 | [0.205-0.265] | 0.24 | [0.211-0.272] |
| DCA | | | | |
| (TDCA + TCDCA)/ | 0.11 | [0.101-0.119] | 0.0968 | [0.0875-0.107] |
| (GDCA + GCDCA) | | | | |
| (TUDAC + GUDCA)/ | 0.132 | [0.106-0.165] | 0.118 | [0.0925-0.151] |
| UDCA | | | | |
| DCA/(LCA + UDCA) | 2.59 | [2.15-3.13] | 2.25 | [1.81-2.78] |
| GCA/GCDCA | 1.15 | [1.05-1.26] | 1.23 | [1.13-1.35] |
| GCA/GDCA | 2.47 | [2.12-2.89] | 2.41 | [2.01-2.88] |
| GCDCA/CDCA | 0.744 | [0.635-0.872] | 0.657 | [0.572-0.755] |
| GCDCA/GDCA | 2.15 | [1.85-2.51] | 1.95 | [1.66-2.29] |
| GCDCA/GLCA | 28.5 | [24.2-33.4] | 21.9 | [18.8-25.6] |
| GDCA/DCA | 0.202 | [0.179-0.229] | 0.208 | [0.186-0.233] |
| GDCA/GLCA | 13.2 | [11.1-15.7] | 11.3 | [9.37-13.5] |
| GLCA/CDCA | 0.0262 | [0.021-0.0326] | 0.0299 | [0.0248-0.0362] |
| GUDCA/UDCA | 0.126 | [0.1-0.158] | 0.112 | [0.0874-0.143] |
| T-a-MCA/CDCA | 0.0209 | [0.0161-0.027] | 0.0246 | [0.0196-0.031] |
| TCDCA/CDCA | 0.0751 | [0.0608-0.0927] | 0.0572 | [0.0469-0.0699] |
| TCDCA/GCDCA | 0.101 | [0.0915-0.111] | 0.0871 | [0.0779-0.0974] |
| TDCA/DCA | 0.0256 | [0.0212-0.0308] | 0.0231 | [0.0188-0.0283] |
| TDCA/GDCA | 0.126 | [0.114-0.14] | 0.111 | [0.0976-0.126] |
| TUDCA/UDCA | 0.00389 | [0.00296-0.00513] | 0.00396 | [0.003-0.00521] |
| UDCA/CDCA | 0.658 | [0.531-0.816] | 0.721 | [0.588-0.885] |
| w-MCA/T-a-MCA | 3.73 | [2.74-5.08] | 3.49 | [2.53-4.81] |
| w-MCA/UDCA | 0.118 | [0.092-0.152] | 0.119 | [0.09-0.158] |
| Steroids | | | | |
| CRTL | 13.4 | [12.6-14.4] | 14.4 | [13.7-15.2] |
| CRTN | 3.89 | [3.67-4.13] | 4.37 | [4.16-4.6] |
| CRCTN | 0.373 | [0.338-0.412] | 0.407 | [0.37-0.449] |
| 11-Deoxy-CTRL | 0.0451 | [0.0414-0.0491] | 0.0525 | [0.0484-0.057] |
| TEST | 0.0288 | [0.0234-0.0355] | 0.045 | [0.0368-0.0551] |
| 17OH-PROG | 0.0433 | [0.0395-0.0476] | 0.0464 | [0.0426-0.0505] |

Bile acids. While few differences were observed in plasma and CSF bile acid levels between control and AD subjects, numerous differences were present in the specific bile acid ratios (Table 10). FIG. 19 shows bile acids metabolic pathway together with their median plasma levels to help understand the biological aspects of specific bile acid ratios. In plasma, the AD group was characterized by lower levels of cholic acid (CA), a product of the neutral bile acids synthesis pathway, while chenodeoxycholic acid (CDCA), a product of the acidic pathway was unchanged. This difference becomes even more pronounced when looking at the CA/CDCA ratio. On the other hand, the difference between neutral and acidic pathway was not present in downstream metabolites, when comparing the secondary unconjugated bile acids ratio, like deoxycholic acid/(lithocholic acid+ursodeoxycholic acid) (DCA/(LCA+UDCA)) or the most abundant primary conjugated derivatives glycocholic acid/glycochenodeoxycholic acid (GCA/GCDCA); Table 8. Of note, small differences between the neutral and acidic pathway were observed in the low abundance taurine conjugates of the secondary bile acids taurodeoxycholic/taurolithocholic acid (TDCA/TLCA). Difference in conjugation ratio (more conjugates than the substrate) was observed in the neutral synthesis pathway (GDCA/DCA and GCA/CA) but not in the acidic synthesis pathway. Differences between the neutral and acidic synthesis pathway were also observed in the conversion of the primary to secondary bile acids. The ratio of downstream products to their precursor in the neutral pathway was higher in the AD group in the case of DCA/CA, TDCA/CA, and GDCA/CA, but not in parallel acidic pathway metabolites (i.e., LCA/CDCA, UDCA/CDCA).

CSF manifested few differences in bile acids and their ratios. The AD group had 1.3-fold higher levels of GLCA and 1.4-fold higher level of T-a-MCA. Additionally, AD group had lower ratio of GCDCA/GLCA (1.3-folds, Table 9).

Steroids. Of those measured, only a few steroid hormones showed different levels between AD and the control. In plasma, dehydroepiandrosterone sulfate (DHEAS) and progesterone were lower in AD group (1.9 and 1.7-folds respectively). Additionally, testosterone and the testosterone/progesterone ratio showed significant gender x group interaction. Females AD subjects showed 1.4-fold lower testosterone, when compared to female controls, but no differences were observed in males. On the other hand, the testosterone/progesterone ratio was 2-fold higher in AD male subjects compared male controls. Testosterone/progesterone ratio differences were not observed in females.

In CSF, only corticosterone showed a significant difference between AD and the control group, however, the magnitude of the-fold difference only ˜1.1.

TABLE 10
Differences in bile acid metabolites and their specific ratios between
control and AD group

| | | Mean (95% CI) | |
|---|---|---|---|
| Metabolite | P-value | Control | AD |

Neutral vs. acidic synthesis pathway

| | | | | | |
|---|---|---|---|---|---|
| CA | 0.0321 | 27.4 | (20.8-26.2) | 19.3 | (13.6-27.4) |
| CDCA | 0.987 | 38.7 | (29.2-51.5) | 46.7 | (32.6-66.9) |
| CA/CDCA | 0.0008 | 0.707 | (0.571-0.876) | 0.363 | (0.271-0.487) |
| GCDCA/ | 0.5 | 2.08 | (1.66-2.61) | 1.97 | (1.51-2.57) |
| GDCA | | | | | |
| TDCA/ | 0.0114 | 1.33 | (1.1-1.62) | 1.81 | (1.49-2.21) |
| TLCA | | | | | |

Conjugation, neutral synthesis pathway

| | | | | | |
|---|---|---|---|---|---|
| GDCA/DCA | 0.0031 | 0.719 | (0.604-0.856) | 1.05 | (0.836-1.33) |
| TDCA/DCA | 0.1 | 0.0605 | (0.0467-0.0783) | 0.0777 | (0.0556- |
| | | | | | 0.109) |
| GCA/CA | 0.0104 | 2.52 | (1.86-3.39) | 4.13 | (2.84-6.01) |
| TCA/CA | 0.076 | 0.506 | (0.358-0.716) | 0.711 | (0.434-1.16) |

Conjugation, acidic synthesis pathway

| | | | | | |
|---|---|---|---|---|---|
| GUDCA/ | 0.7 | 0.546 | (0.341-0.876) | 0.515 | (0.334-0.793) |
| UDCA | | | | | |
| TUDCA/ | 0.746 | 2.83 | (1.93-4.17) | 2.04 | (1.33-3.13) |
| UDCA | | | | | |
| GCDCA/ | 0.491 | 8.15 | (6.21-10.7) | 8.36 | (5.68-12.3) |
| CDCA | | | | | |
| TCDCA/ | 0.644 | 0.734 | (0.53-1.01) | 0.687 | (0.433-1.09) |
| CDCA | | | | | |
| TLCA/LCA | 0.961 | 0.282 | (0.2-0.396) | 0.252 | (0.182-0.347) |
| GLCA/LCA | 0.721 | 0.711 | (0.53-0.955) | 0.749 | (0.552-1.02) |

Conversion of primary to secondary, gut metagenome activity

| | | | | | |
|---|---|---|---|---|---|
| DCA/CA | 0.0459 | 8.01 | (5.8-11.1) | 12.0 | (7.89-18.1) |
| TDCA/CA | 0.0056 | 0.469 | (0.309-0.711) | 0.906 | (0.517-1.59) |
| GDCA/CA | 0.0007 | 5.76 | (4.01-8.27) | 12.5 | (7.71-20.2) |
| LCA/CDCA | 0.451 | 0.66 | (0.419-1.04) | 0.589 | (0.42-0.825) |
| UDCA/ | 0.691 | 1.39 | (0.815-2.36) | 1.94 | (1.23-3.07) |
| CDCA | | | | | |
| TUDCA | 0.0275 | 1.07 | (0.78-1.48) | 1.92 | (1.53-2.41) |

Means are expressed in nM or as a ratio of the concentrations.
Metabolites and their ratios are stratified by the metabolic affiliations.
All tested bile acids and their ratios are presented in Table 6.

Relation between CSF and plasma AD markers: In the current study, matched plasma and CSF samples were collected, allowing an assessment of the relationships between metabolites in these pools. Spearman's p rank order correlation between plasma and CSF lipid mediator levels are shown in Table 11. The associations were distinct by metabolite classes, with oxylipins showing only 2 of 15 significant correlations, while bile acids and steroids showing 14 of 18 significant correlations. Correlations within PUFA and PUFA ethanolamide were also apparent for the long chain omega 3 species (DHA EPA and DHA ethanolamide) but not others.

TABLE 11
Spearman's ρ rank order correlation between plasma and CSF metabolites.

| Metabolite class | Metabolite | Spearman's p | P-value |
|---|---|---|---|
| Oxylipins | PGF2a | −0.2 | 0.0746 |
| | F2-IsoP | 0.34 | 0.0019 |
| | 12_12-DiHOME | 0.26 | 0.0233 |
| | 9_10-DiHOME | 0.15 | 0.1810 |
| | 15_16-DiHODE | 0.21 | 0.0625 |
| | 14_15-DiHETrE | 0.062 | 0.5830 |
| | 11_12-DiHETrE | 0.11 | 0.3340 |
| | 17_18-DiHETE | 0.33 | 0.0027 |
| | 19_20-DiHOPE | 0.16 | 0.1450 |
| | 13-HODE | 0.029 | 07970 |
| | 9-HODE | −0.054 | 0.6380 |
| | 13-HOTE | −0.014 | 0.9010 |
| | 9-HOTE | −0.051 | 0.6520 |
| | 12(13)-EpOME | 0.0089 | 0.9380 |
| | 9(10)-EpOME | −0.028 | 0.8070 |
| Acyl-EA | OEA | −0.031 | 0.7820 |
| | LEA | 0.062 | 0.5860 |
| | DHEA | 0.51 | 0.0001 |
| PUFA | LA | 0.15 | 0.1860 |
| | ALA | 0.2 | 0.0826 |
| | AA | −0.049 | 0.6670 |
| | EPA | 0.45 | 0.0001 |
| | DHA | 0.42 | 0.0001 |
| Bile Acids | CDCA | 0.51 | 0.0001 |
| | UDCA | 0.78 | 0.0001 |
| | DCA | 0.71 | 0.0001 |
| | TCA | 0.62 | 0.0001 |
| | TCDCA | 0.43 | 0.0001 |
| | TUDCA | 0.16 | 0.157 |
| | TDCA | 0.54 | 0.0001 |
| | GCA | 0.36 | 0.0012 |
| | GCDCA | 0.19 | 0.0925 |
| | GUDCA | 0.47 | 0.0001 |
| | GDCA | 0.54 | 0.0001 |
| | GLCA | −0.083 | 0.4670 |
| Steroids | 17OH-PROG | 0.58 | 0.0001 |
| | Cortisol | 0.3 | 0.0071 |
| | Cortexolone | 0.089 | 0.4340 |
| | Corticosterone | 0.51 | 0.0001 |
| | Testosterone | 0.81 | 0.0001 |

Significant p-values are bolded.

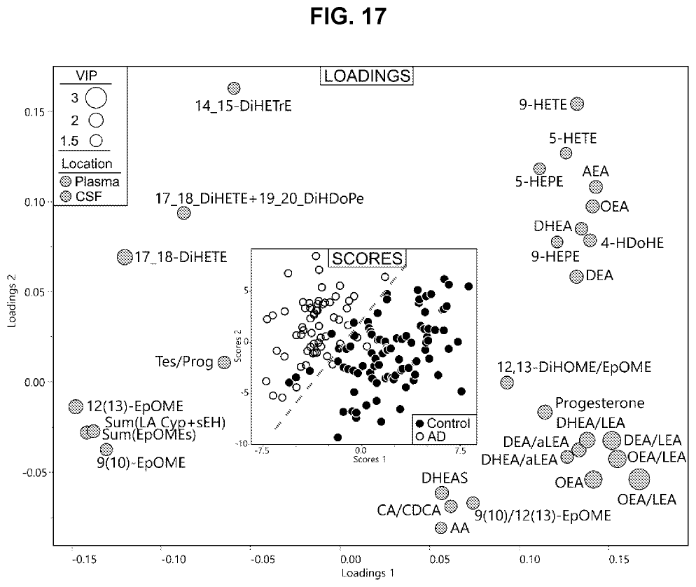

Next, partial least square discriminant analysis (PLS-DA) was used to illustrate the relationship between plasma and CSF AD markers (FIG. 17). That discrimination between control and AD was dominated by the plasma metabolites. Fifteen plasma metabolites (and their ratios) manifested variable importance in projection (VIP) score >1.4 compared to only 4 CSF metabolites. The discrimination between AD and the control group was characterized by higher plasma 17,18-DiHETE (VIP=2.16) and CSF EpOMEs (VIP=1.95 and 1.58 for the 12(13) and 9(10) isoforms respectively) and lower levels of the acylethanolamide ratios including both DHEA/LEA and DEA/LEA in plasma and both plasma and CSF OEA/LEA. Plasma and CSF OEA/LEA manifest similar discriminatory power based on their proximity on the loading plot. On the other hand, plasma 17,18-DiHETE and CSF EpOMEs occupied distinct parts of the loading plot, suggesting distinct discriminatory properties. The VIPs for each metabolite are provided in Table 12.

TABLE 12
Variable importance in projection (VIP) scores for all plasma and CSF
variables used for partial least square discriminant analysis (PLS-DA)

| Variable | Tissue | VIP |
|---|---|---|
| OEA/LEA | Plasma | 2.9 |
| DEA/LEA | Plasma | 2.54 |
| CSF_OEA/LEA | CSF | 2.48 |
| CSF_OEA | CSF | 2.44 |
| DHEA/LEA | Plasma | 2.18 |
| 17_18-DiHETE | Plasma | 2.16 |
| Progesterone | Plasma | 1.97 |
| CSF_12(13)-EpOME | CSF | 1.95 |
| DEA/aLEA | Plasma | 1.95 |
| DEA | Plasma | 1.79 |
| DHEAS | Plasma | 1.79 |
| OEA | Plasma | 1.77 |
| AEA | Plasma | 1.75 |
| CSF_Sum(EpOMEs) | CSF | 1.74 |
| CSF_Sum(LA Cyp + sEH) | CSF | 1.74 |
| 17_18_DiHETE + 19_20_DiHDoPe | Plasma | 1.74 |
| 9-HETE | Plasma | 1.74 |
| 12,13-DiHOME/EpOME 2 | Plasma | 1.71 |
| 4-HDoHE | Plasma | 1.71 |
| DHEA/aLEA | Plasma | 1.69 |
| Tes/Prog | Plasma | 1.67 |
| CSF_9(10)/12(13)-EpOME | CSF | 1.64 |
| DHEA | Plasma | 1.64 |
| CA/CDCA | Plasma | 1.6 |
| CSF_9(10)-EpOME | CSF | 1.58 |
| 14_15-DiHETrE | Plasma | 1.55 |
| 9-HEPE | Plasma | 1.47 |
| 5-HETE | Plasma | 1.45 |
| 5-HEPE | Plasma | 1.45 |
| CSF_AA_RelAb | CSF | 1.43 |
| 12-HEPE | Plasma | 1.39 |
| Sum(HDoHEs) | Plasma | 1.38 |
| 1-OG | Plasma | 1.37 |
| CSF_GLCA | CSF | 1.36 |
| 9_12_13-TriHOME | Plasma | 1.36 |
| DGLEA | Plasma | 1.35 |
| Sum(DiHOME)/Sum(EpOME) | Plasma | 1.34 |
| 11_12-DiHETrE | Plasma | 1.34 |
| CSF_PGF2a | CSF | 1.31 |
| CSF_12,13-DiHOME/EpOME | CSF | 1.3 |
| 8-HETE | Plasma | 1.3 |
| 2-OG | Plasma | 1.29 |
| 15_16-DiHODE/15(16)-EPODE | Plasma | 1.28 |
| (GDCA + GLCA)/(TDCA + TLCA) | Plasma | 1.28 |
| 11-HETE | Plasma | 1.27 |
| CSF_GCDCA/GLCA | CSF | 1.26 |
| GDCA/CA | Plasma | 1.26 |
| CSF_LA_RelAb | CSF | 1.25 |
| 14-HDoHE | Plasma | 1.25 |
| 9_10-e-DiHO | Plasma | 1.23 |
| 12-HETE | Plasma | 1.22 |
| CSF_F2-IsoP | CSF | 1.18 |
| F2-IsoP | Plasma | 1.16 |
| 15-HEPE | Plasma | 1.16 |
| 1-LG | Plasma | 1.16 |
| CSF_(GDCA + GLCA)/(TDCA) | CSF | 1.14 |
| Sum(DiHODE)/Sum(EpODE) | Plasma | 1.13 |
| CSF_UDCA | CSF | 1.12 |
| CA | Plasma | 1.12 |
| Testosterone | Plasma | 1.12 |
| GDCA/DCA | Plasma | 1.11 |
| w-MCA/UDCA | Plasma | 1.1 |
| CSF_Sum(DiHOME)/Sum(EpOME) | CSF | 1.09 |
| CSF_TEST | CSF | 1.09 |
| 15-HETE | Plasma | 1.09 |
| 2-LG | Plasma | 1.09 |
| CSF_14,15/17,18-DiHETE | CSF | 1.07 |
| (TDCA + GDCA)/DCA | Plasma | 1.07 |
| LEA | Plasma | 1.07 |
| 13-HODE | Plasma | 1.06 |
| TUDCA | Plasma | 1.06 |
| CSF_UDCA/CDCA | CSF | 1.03 |
| CSF_EPA_RelAb | CSF | 1.03 |
| PGF2a | Plasma | 1.03 |
| (w) + a + b-MCA | Plasma | 1.03 |
| AA_screen | Plasma | 1.02 |
| CSF_TCDCA/GCDCA | CSF | 1.01 |
| CSF_11-Deoxy-CTRL | CSF | 1.01 |
| LCA/CDCA | Plasma | 1.01 |
| PGE2 | Plasma | 1 |
| 12_13-DiHOME | Plasma | 0.999 |
| CSF_9_10-DiHOME | CSF | 0.986 |
| CSF_11,12/14,15-DiHETrE | CSF | 0.975 |
| CSF_14,15/11,12-DiHETrE | CSF | 0.971 |
| CSF_GUDCA/UDCA | CSF | 0.971 |
| 9,10-DiHOME/EpOME 2 | Plasma | 0.967 |
| GCA/CA | Plasma | 0.966 |
| 12-HEPE/12-HETE | Plasma | 0.964 |
| CSF_(TUDAC + GUDCA)/UDCA | CSF | 0.962 |
| 9-HODE | Plasma | 0.951 |
| (GCA + TCA)/CA | Plasma | 0.95 |
| 9(10)-EpOME | Plasma | 0.948 |
| TDCA/CA | Plasma | 0.943 |
| LA_screen | Plasma | 0.941 |
| CSF_TDCA/GDCA | CSF | 0.938 |
| GHDCA | Plasma | 0.917 |
| EPEA_Screen | Plasma | 0.912 |
| CSF_(TDCA + TCDCA)/(GDCA + GCDCA) | CSF | 0.881 |
| CSF_9,10-DiHOME/EpOME | CSF | 0.867 |
| GLCA | Plasma | 0.866 |
| TXB2 | Plasma | 0.86 |
| TDCA/TLCA | Plasma | 0.839 |
| (TUDAC + GUDCA)/UDCA | Plasma | 0.836 |
| PGD2 | Plasma | 0.835 |
| CSF_TCDCA | CSF | 0.799 |
| 5_6-DiHETrE | Plasma | 0.791 |
| 12_13-DiHODE | Plasma | 0.788 |
| CSF_GLCA/CDCA | CSF | 0.785 |
| CSF_GCDCA/GDCA | CSF | 0.784 |
| CSF_T-a-MCA | CSF | 0.777 |
| CSF_GDCA | CSF | 0.774 |
| CSF_DCA | CSF | 0.77 |
| CSF_20-HETE | CSF | 0.768 |
| NA-Gly | Plasma | 0.768 |
| GCA/GCDCA | Plasma | 0.766 |
| GDCA | Plasma | 0.766 |
| CSF_DHA_RelAb | CSF | 0.759 |
| CSF_GCA/GDCA | CSF | 0.758 |
| w-MCA/T-a-MCA | Plasma | 0.757 |
| CSF_9,10/12,13-DiHOME | CSF | 0.752 |
| DCA/(LCA + UDCA) | Plasma | 0.747 |
| GLCA/CDCA | Plasma | 0.733 |
| TLCA/CDCA | Plasma | 0.724 |
| ALA screen | Plasma | 0.723 |
| 14,15/11,12-DiHETrE | Plasma | 0.72 |
| 11,12/14,15-DiHETrE | Plasma | 0.72 |
| TCA/CA | Plasma | 0.72 |
| CSF_17_18-DiHETE | CSF | 0.719 |
| CSF_13-HOTE | CSF | 0.715 |
| 2-AG | Plasma | 0.713 |
| 9_10-DiHODE/9(10)-EpODE | Plasma | 0.711 |
| GCA/GDCA | Plasma | 0.705 |
| 8_9-DiHETrE | Plasma | 0.705 |
| CSF_TCDCA/CDCA | CSF | 0.704 |
| DHA_screen | Plasma | 0.703 |
| EPA_screen | Plasma | 0.701 |
| 15(16)-EpODE | Plasma | 0.695 |
| 5_15-DiHETE | Plasma | 0.681 |
| 11(12)-EpETrE | Plasma | 0.679 |
| UDCA/CDCA | Plasma | 0.675 |
| DCA/CA | Plasma | 0.673 |
| CSF_CRTL | CSF | 0.659 |
| NO-Gly | Plasma | 0.656 |
| 1-AG | Plasma | 0.653 |
| CSF_DHEA | CSF | 0.644 |
| CSF_DCA/(LCA + UDCA) | CSF | 0.643 |
| 17-OH Prog | Plasma | 0.634 |
| 13-KODE | Plasma | 0.621 |
| POEA_Screen | Plasma | 0.62 |
| 12(13)-EpOME | Plasma | 0.619 |
| CSF_TUDCA/UDCA | CSF | 0.616 |
| CSF_19_20-DiHDoPE | CSF | 0.616 |
| CSF_(GDCA + TDCA)/(TLCA+GLCA) | CSF | 0.613 |
| 9-HOTE | Plasma | 0.61 |
| GCDCA/CDCA | Plasma | 0.604 |
| 9_10-DiHOME | Plasma | 0.603 |
| CSF_ALA_RelAb | CSF | 0.599 |
| CSF_14_15-DiHETE | CSF | 0.588 |
| CSF_TDCA/DCA | CSF | 0.586 |
| CSF_TDCA/DCA 2 | CSF | 0.586 |
| CSF_LEA | CSF | 0.583 |
| CSF_EPA + DHA diols | CSF | 0.582 |
| CSF_CRCTN | CSF | 0.582 |
| CSF_GDCA/GLCA | CSF | 0.581 |
| CSF_CRTN | CSF | 0.58 |
| CSF_Sum(DiHOMEs) | CSF | 0.575 |
| (TCDCA + GCDCA)/CDCA | Plasma | 0.574 |
| GCDCA | Plasma | 0.574 |
| GCDCA/GLCA | Plasma | 0.573 |
| CSF_w-MCA/UDCA | CSF | 0.57 |
| 11(12)/14(15)-EpETrE | Plasma | 0.565 |
| LCA | Plasma | 0.563 |
| CSF_a-MCA | CSF | 0.562 |
| (TDCA + TCDCA)/(GDCA + GCDCA) | Plasma | 0.541 |
| (GDCA + TDCA)/(TUDCA + GUDCA) | Plasma | 0.53 |
| TLCA | Plasma | 0.528 |
| 13-HOTE | Plasma | 0.51 |
| TDCA/GDCA | Plasma | 0.508 |
| CSF_12_13-DiHOME | CSF | 0.507 |
| 9(10)-EPODE | Plasma | 0.506 |
| TCDCA/GCDCA | Plasma | 0.503 |
| CSF_DHEA/LEA | CSF | 0.496 |
| CSF_(GDCA + TDCA)/(TUDCA + GUDCA) | CSF | 0.495 |
| CSF_w-MCA | CSF | 0.493 |
| Cortisone | Plasma | 0.487 |
| CSF_13-HODE | CSF | 0.477 |
| CSF_T-a-MCA/CDCA | CSF | 0.455 |
| TDCA/DCA | Plasma | 0.443 |
| CSF_TUDCA | CSF | 0.442 |
| TCA | Plasma | 0.437 |
| T-a-MCA/CDCA | Plasma | 0.436 |
| CSF_9-HODE | CSF | 0.434 |
| UDCA | Plasma | 0.397 |
| aLEA | Plasma | 0.395 |
| (GLCA + TLCA)/LCA | Plasma | 0.39 |
| w + a + (b)-MCA | Plasma | 0.373 |
| TCDCA/CDCA | Plasma | 0.371 |
| GUDCA | Plasma | 0.369 |
| CDCA | Plasma | 0.368 |
| CSF_(TCDCA + GCDCA)/CDCA | CSF | 0.345 |
| 19_20-DiHDoPE | Plasma | 0.343 |
| CSF_15_16-DiHODE | CSF | 0.335 |
| GDCA/GLCA | Plasma | 0.331 |
| GCA | Plasma | 0.329 |
| CSF_GCDCA/CDCA | CSF | 0.323 |
| TDCA | Plasma | 0.323 |
| CSF_11_12-DiHETrE | CSF | 0.309 |
| Cortisol | Plasma | 0.302 |
| GCDCA/GDCA | Plasma | 0.299 |
| (GDCA + TDCA)/(TLCA + GLCA) | Plasma | 0.29 |
| CSF_GCDCA | CSF | 0.273 |
| 15_16-DiHODE | Plasma | 0.269 |
| Cortexolone | Plasma | 0.263 |
| CSF_GUDCA | CSF | 0.247 |
| (TCA + GCA + TDCA + GDCA)/(GUDCA + | Plasma | 0.247 |
| TUDCA + GLCA + TLCA + TCDCA + GCDCA) | | |
| T-w + (a) + b-MCA | Plasma | 0.247 |
| CSF_TDCA | CSF | 0.222 |
| CSF_9-HOTE | CSF | 0.218 |
| 9_10-DiHODE | Plasma | 0.214 |
| CSF_GCA | CSF | 0.213 |
| 14_15-DiHETrE/14(15)-EpETrE | Plasma | 0.213 |
| CSF_CDCA | CSF | 0.196 |
| 14(15)-EpETrE | Plasma | 0.174 |
| CSF_GDCA/DCA | CSF | 0.158 |
| CSF_(TDCA + GDCA)/DCA | CSF | 0.156 |
| DCA | Plasma | 0.146 |
| TCDCA | Plasma | 0.13 |
| CSF_GCA/GCDCA | CSF | 0.122 |
| corticosterone | Plasma | 0.12 |
| 13-KODE/13-HODE | Plasma | 0.113 |
| CSF_w-MCA/T-a-MCA | CSF | 0.111 |
| CSF_14_15-DiHETrE | CSF | 0.109 |
| CSF_17OH-PROG | CSF | 0.106 |
| CSF_TCA | CSF | 0.0631 |
| Sum(DiHETrE/EpETrE) | Plasma | 0.0621 |

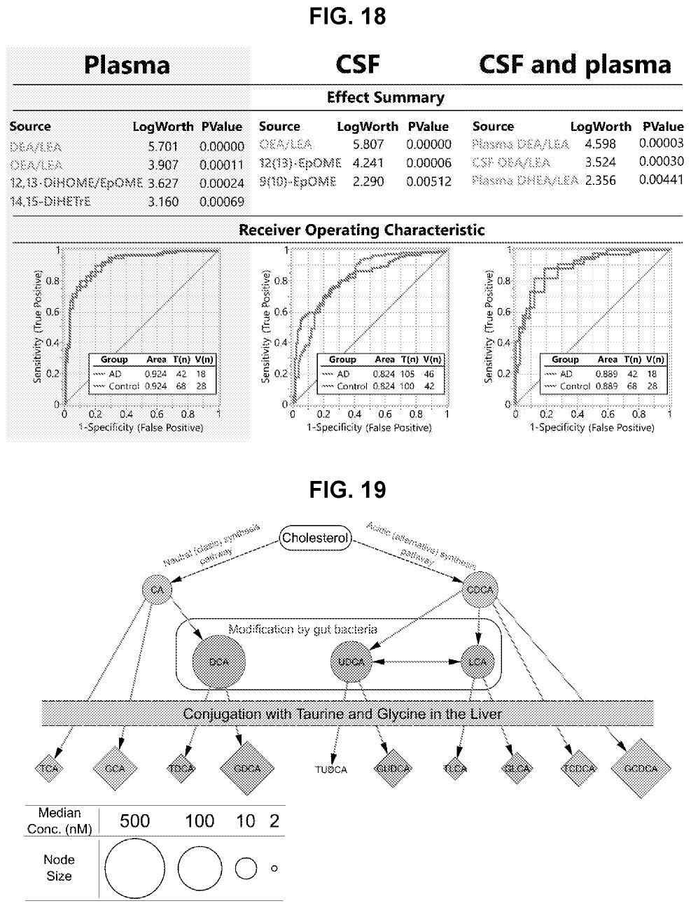

Fatty acid ethanolamides and CYP/sEH metabolites are strong AD predictors in both plasma and CSF. Fatty acid ethanolamides and CYP/sEH metabolites are strong AD predictors in both plasma and CSF. Predictive modeling was used to investigate how well plasma and CSF metabolites can report AD status. Plasma lipid mediators generated stronger models than those in CSF with area under the receiver operator characteristic curves (ROC AUC) of 0.924 vs. 0.824, with the two models consisting of distinct metabolites (FIG. 18). However, in both matrices, the strongest predictors belonged to the same two metabolic pathways, the acyl ethanolamides and CYP/she pathway. Plasma predictors included ethanolamides (OEA and DEA normalized to the LEA level), the 12,13-DiHOME/EpOME an indicator of sEH activity and sEH metabolite of AA (14,15-DiHETrE). In CSF, the strongest predictors included OEA/LEA and the linoleate-derived epoxides 12(13)-EpOME and 9(10)-EpOME. When plasma and CSF markers were combined in predictive model efforts, the resulting model consisted uniquely of ethanolamides, including plasma long chain PUFA ethanolamides (DEA/LEA and DHEA/LEA) and CSF OEA/LEA. This model resulted in the ROC AUC of 0.889.

For all 3 models, ethanolamides OEA, DEA and DHEA were stronger predictors when used as a ratio to LA derived ethanolamide—LEA. LEA itself was not different between AD and the control group in either plasma or CSF (FIG. 15 and FIG. 16) unlike OEA, DEA and DHEA. Therefore, LEA likely serves as a surrogate for the general acyl-ethanolamides level and adjustment of other ethanolamides by LEA lowers intra-individual variability.

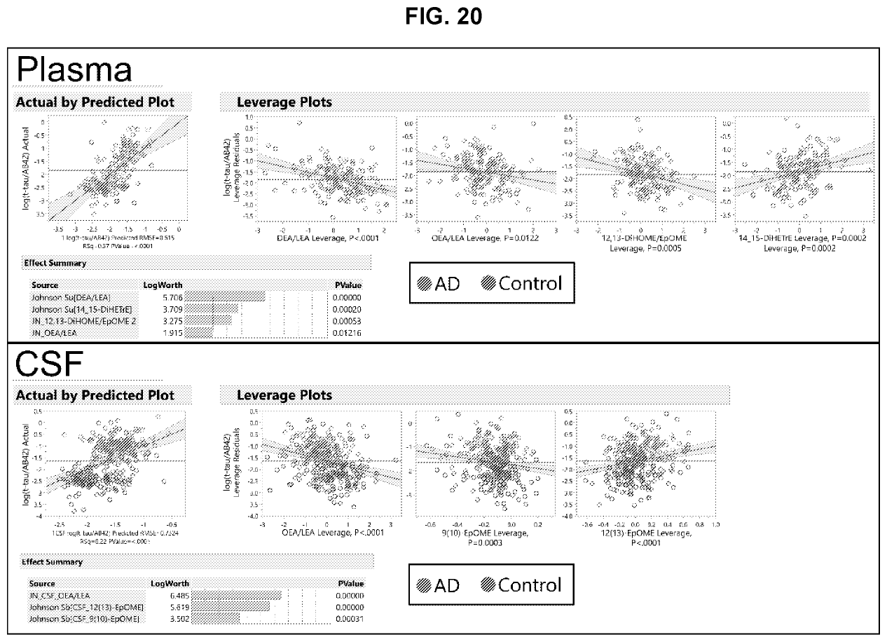

To investigate relevance of identified metabolites to the progression of AD pathology, components of the predictive models were applied to a linear model for log(t-Tau/AB42), as a maker of AD pathology. The AD predictive model components produced strong linear regression models with log(t-Tau/AB42) in both plasma (r²=0.37, p value <0.0001), and CSF (r²=0.22, p value <0.0001) shown in FIG. 20, further supporting the approach towards fasting state stratification.

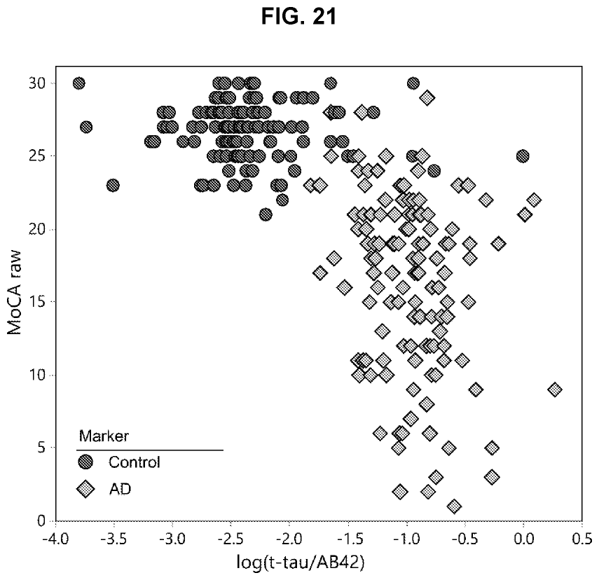

Lipid mediator-cognitive score associations in AD. The AD cohort is characterized by a high log(t-Tau/Aβ42) ratio and MoCA scores ranging from normal cognitive function to severe cognitive impairment (FIG. 21). Taking advantage of the broad MoCA range, lipid mediator associations with cognitive function in this group of pathological levels of t-Tau/Aβ42 were investigated. Additionally, since the AD cohort was represented by subjects in both fasted and non-fasted states, the analysis was stratified by fasting state for plasma samples (Table 13). In the fasting state, PUFA oxidation markers, 5,15-DiHETE and 9-HETE were negatively associated with the MoCA score (although only 5,15-DiHETE passed FDR correction). 5,15-DiHETE can have enzymatic or autooxidative origin, whereas 9-HETE is a strictly an autooxidative product. 5,15-DilHETE correlated with 9-HETE in fasted subjects with an R²=0.415 (n=60; p<0.001). In non-fasted AD subjects, a strong positive association between the MoCA score and EPA-derived ethanolamide (EPEA) as well as the levels of EPA and DHA were observed. Additionally, a positive correlation was detected between MoCA and the EPA-derived 17,18-DiHETE, the DHA-derived 14-HDoHE and the 18 carbon PUFAs (LA and ALA), however, these did not pass FDR correction.

In CSF, the linoleic acid derived epoxides 12(13)- and 9(10)-EpOMEs showed weak but significant positive correlations with MoCA (p >0.2, p<0.005; Table 14. Additionally, positive associations were observed between MoCA and DHA and DHA derived diol (19,20-DiHDoPE) and conjugated bile acids GCA (and the ratio of GCA to GDCA and GCDCA), TCDCA and the conjugated to unconjugated ratio for DCA and CDCA (GCA/GCDCA, GCDCA/CDCA and TCDCA/CDCA). However, only linoleic acid epoxides passed the FDR correction.

Additionally, utilizing subjects from both AD patients and healthy controls, associations of other components of AD pathology, including log(t-Tau/AB42), t-Tau, AB42, p-Tau, p-Tau/t-Tau and MoCA were investigated with plasma and CSF metabolites.

TABLE 13
Spearman's ρ rank order correlation between MoCA score and plasmid
lipid mediators of the AD patients.

| | Fasted | | | Non-Fasted | |
|---|---|---|---|---|---|
| | Spearman's | P- | | Spearman's | P- |
| Metabolite | ρ | Value | Metabolite | ρ | Value |
| 5,15- | −0.448 | 0.0005 | EPEA | 0.424 | 0.0003 |
| DIHETE | | | | | |
| 9HETD | −0.338 | 0.0102 | EPA | 0.386 | 0.001 |
| 13-KODE | −0.299 | 0.0238 | DHA | 0.338 | 0.0043 |
| DCA | −0.273 | 0.0398 | 17,18- | 0.3 | 0.0117 |
| | | | DHETE | | |
| | | | 4-HDoHE | 0.269 | 0.0246 |
| | | | LA | 0.267 | 0.0254 |
| | | | ALA | 0.249 | 0.0373 |
| | | | 9-HETE | −0.246 | 0.0405 |
| | | | 8-HETE | −0.24 | 0.0459 |

Analysis stratified by predicted fasted state.
Only correlations with p < 0.05 are shown.
P-values that passed FDR correction at q = 0.2 are bolded.

TABLE 14
Spearman's ρ rank order correlation between MoCA
score and plasmid lipid mediators of the AD patients.

| Metabolite | Spearman's ρ | P-Value |
|---|---|---|
| 12(13)-EpOME | 0.279 | 0.0009 |
| 9(10)-EpOME | 0.24 | 0.0047 |
| 19_20-DiHOPE | 0.21 | 0.0138 |
| GCA | 0.207 | 0.0152 |
| DHA | 0.203 | 0.0174 |
| GCA/GDCA | 0.202 | 0.0182 |
| GCDCA/CDCA | 0.19 | 0.026 |
| TDCA/DCA | 0.19 | 0.0261 |
| GCA/GDCA | 0.18 | 0.0352 |
| TCDCA/CDCA | 0.172 | 0.0441 |
| TCDCA | 0.17 | 0.0468 |

Only correlations with p < 0.05 are shown.
P-values that passed FDR correction at q = 0.2 are bolded.

Metabolic disruptions influencing vascular physiology, inflammation and energy metabolism have been reported to increase the risk of Alzheimer's disease, however whether these changes are independent risk factors or how they may interact has not been well established. If novel biomarkers of AD can be identified within these domains, they could not only provide useful screening and risk assessment tools but may also provide insight into connections between metabolism and neurodegenerative diseases. To this end, a comprehensive analysis of plasma and CSF lipid mediators, endogenous regulators of multiple processes, including inflammation and energy metabolism, was performed, and their associations with AD and cognitive function were described. In the process, clear differences between AD and healthy controls in two metabolic pathways were identified, and also CYP/sEH and fatty acids-derived ethanolamides, and subtle differences in bile acids and steroids were observed. The potency of identified markers to predict AD is comparable with other plasma and CSF proteomic biomarkers.

AD-associated differences in plasma bile acid agreed with previously reported analyses of The Religious Orders Study and the Rush Memory and Aging Project (ROS/MAP) cohort. These included lower levels of CA and the CA:CDCA ratio in AD, suggesting that the neutral bile acid synthesis pathway could be affected in AD. There are few studies regarding the shift between neutral and acidic BA synthesis, one of them reporting an increase in neutral/acidic pathway products ratio in nonalcoholic steatohepatitis. However, the biological relevance of this difference in terms of AD is yet to be determined. Interestingly, associations between postprandial bile acids and cognition have been previously reported, with few associations in the fasting state. Considering that the current disclosure focuses in part on AD related differences in the fasting state, further studies probing postprandial bile acid metabolism and AD are suggested. Interestingly, several bile acid and steroid differences in AD were gender specific. Few studies report gender specific action of TUDCA and UDCA on ER stress markers in rodent model for the prion disease. These findings further support the importance of gender focused approaches when investigating cholesterol-derived metabolism in the context of AD, as established with regards to the links between ApoE4 and AD risk in post-menopausal women.

With respect to fatty acid metabolism, this study identified substantial AD-associated elevations in CYP/sEH pathway products and lower levels of acylethanolamides in both plasma and CSF, although different elements of these pathways were affected in plasma and CSF. Oxylipin and endocannabinoid levels show no association between plasma and CSF, suggesting independent regulation of these pools. Likewise, CYP/sEH metabolites of plasma and CSF manifest distinct discriminatory power in PLS-DA models of AD. On the other hand, CSF and plasma acylethanolamides seem to manifest similar AD discriminatory power and can be substituted in the AD predictive model. These findings are consistent with previous reports implicating both CYP/sEH metabolites and acylethanolamides as important regulators of inflammation in neurodegenerative disorders. In the CYP/sEH pathway, polyunsaturated fatty acids are converted to anti-inflammatory and vasodilating epoxy fatty acids by CYPs, which are further metabolized to pro-inflammatory and vasoconstricting diols by sEH, a process primarily recognized in cardiovascular disease. As some ethanolamides, like PEA were reported to be associated with cognition in AD patients in an analysis of a small (n=40) cohort and sEH reports are based mainly on animal models and brain sEH gene expression, this study provides a comprehensive analysis of AD-associated alteration in the levels of the array of these lipid mediators in plasma and CSF.

In the current study, higher plasma levels of the EPA sEH metabolite 17,18-DiHETE in AD patients were found, but only slight difference in the parallel AA metabolites were observed. Notably, EPA metabolites derived from LOX pathways were lower in AD patients, suggesting that the observed differences are not a result of differential omega-3 fatty acid intake, but rather specific enhancement in sEH-dependent EPA metabolism. Omega-3 sEH metabolites are particularly potent regulators of the cardiovascular system, especially blood vessel tone and vascular inflammation and sEH inhibitors have been suggested to improve outcomes for both cardiovascular and neurodegenerative diseases.

Clinical associations between cardiovascular disease and AD have been reported, where the regulation of a vascular tone and blood flow play a role in both pathologies. Additionally, plasma EPA sEH metabolites were previously reported to be negatively associated with perceptual speed in cognitively normal subjects. Therefore, the current findings further support involvement of vascular dysfunction in AD, perhaps through alterations to the blood brain barrier and vascular-related inflammatory signaling, with overlapping molecular mechanisms leading to cardiovascular and neurodegenerative pathologies. When considering these shifts in oxylipin profiles, it is important to remember that an epoxides reservoir is generated by esterification into phospholipid membranes, whereas diols are not readily reincorporated into the membranes and rapidly appear in the free pool and are actively excreted from cells. Since tissue esterified lipid mediators were not evaluated in these samples, it is difficult to know whether the observe difference in EPA diol was due to an increased production of CYP/sEH metabolites or increased clearance of membrane bound EPA epoxides, and future studies are needed to resolve this issue.

In contrast to plasma, CSF showed higher levels of LA-derived epoxides, along with a moderate increase in LA-diols, but not CYP/EH metabolites of longer chain PUFAs. The source of the CSF metabolites is likely tied to the central nervous system, and linoleate-derived oxylipins have been identified as the dominant form in the developing rat brain. Like long chain PUFAs, LA-derived epoxides and diols can also modulate vascular tone and multiple studies point towards their cytotoxic and pro-inflammation nature. However, most of these studies used concentrations greatly exceeding physiological levels and cytotoxic effects were sEH-dependent, pointing towards LA diols as cytotoxic agents. Interestingly, LA CYP/sEH metabolites elevated in the spinal tissue of burn victims were shown to activate the transient vanilloid receptor type 1 (TRPV1). Activation of the TRPV1 can rescue neuronal function from Aβ-induce impairment and can alleviates cognitive and synaptic plasticity impairments in the APP23/PS45 mouse model of AD. Considering that acylethanolamides are also potent activators of the TRPV1, increased LA CYP metabolites may compensate for the AD-related decrease in these ethanolamides. This hypothesis is supported by positive correlation of CSF LA-epoxides with the MoCA score in the AD patients, suggesting elevation of epoxy fatty acids in the central nervous system being potentially beneficial in AD. It is important to mention that the transportation of oxylipins in CSF is poorly understood. In plasma, the majority of oxylipins are transported as complex lipid esters in lipoproteins, with different lipoproteins manifesting distinct oxylipin compositions. Therefore, the potential for lipoprotein-dependent oxylipin transport within CSF specific HDL particles is particularly intriguing and warrants further investigation.

Together the CSF and plasma results implicate changes in both peripheral and central CYP/sEH metabolism in association with AD and cognitive impairment. These conclusions are consistent with previous reports of single nucleotide polymorphisms (SNPs) in the CYP2J2 promoter region that reduces gene expression by ˜50% that appears to increase the ApoE4-independent AD risk. Several functional SNPs are also known to influence sEH activity and/or expression and influence disease risk. Relevant to AD-associated pathologies, loss of function sEH mutations protect neurons from ischemia-induced death and may alter the risk of vascular cognitive impairment. Additionally, postmortem brains from human subjects with AD show higher sEH levels, when compared to the healthy controls, and sEH inhibitors can reverse microglia and astrocyte reactivity and immune pathway dysregulation in mouse AD models. Additionally, brain sEH was positively associated with AD in a replicated protein wide-association study of AD. Therefore, reducing sEH function appears to be protective, and supports sEH as a valuable therapeutic target for the treatment and investigation of neuro-inflammatory pathologies including AD. Both plasma and CSF acylethanolamides were lower in AD, with both PLS-DA and predictive model identifying OEA as the strongest predictor of AD in the current cohort. Acylethanolamides are generally considered anti-inflammatory and neuroprotective and were previously implicated in neuroinflammatory processes. Their neuroprotective action is mediated by activation of the CB1 and CB2 receptors and TRPV1, involved in the acute and inflammatory pain signals in periphery. Some acylethanolamides, like OEA, are also peroxisome proliferator-activated receptor (PPAR) a agonist and regulate satiety and sleep with both central and peripheral anorexigenic effects. Notably, sleep disturbances themselves have been reported to be a risk factor for AD, and the identified reductions in CSF OEA would be consistent with such a physiological manifestation. A recent study also suggested that the EPA-derived ethanolamide (EPEA) is a potential PPAR γ agonist, a transcription factor known for its neuroprotective and anti-inflammatory action.

Interestingly, non-fasting levels of EPEA showed positive association with MoCA in AD patients. This agrees with previous findings of acylethanolamides in non-fasted individuals, including EPEA, being positively associated with perceptual speed in cognitively normal elderly individuals. Literature provides conflicting results regarding both the levels of acylethanolamides in biological fluids, as well as the expression of CB1 and CB2 receptors in the context of AD. Nevertheless, the body of literature suggests that exogenous cannabinoids are potent activators of the CB1 and CB2 receptors with potential therapeutic benefit for AD treatment, due to their neuroprotective and anti-inflammatory activity. The data suggest acylethanolamide biology is altered in relation to both AD pathology as well as cognition. However, future studies are needed to fully elaborate the role of these endocannabinoids in AD pathology.

In conclusion, the current study shows AD related differences in CYP/sEH and acylethanolamide metabolism observed in both plasma and CSF. Strong predictive and discriminant models suggest their potential as biomarkers of AD-associated metabolic disruptions. This further supports the contention that a combination therapy reducing she activity while increasing acylethanolamide tone by either promoting production or reducing degradation could be a more effective strategy than targeting either pathway independently in treating multifactorial inflammatory diseases like AD. Important questions remain regarding the metabolic changes in the lipid mediators preceding pathological changes in tau and cognitive decline. Plasma sEH metabolites of the long chain omega-3 PUFA have previously been reported to be negatively associated, and PUFA ethanolamides positively associated, with perceptual speed, mimicking the currently described AD related associations. While these data suggest early alterations in these important regulatory pathways, a comprehensive analysis of longitudinal metabolome changes in relation to cognition and tauopathies is warranted. Combining assessments of dietary, lifestyle and genetic factors promoting these metabolic changes offers the opportunity for novel risk factor discovery and the development of targeted preventive measures.

### Example 6

### Therapeutic Approaches for Neuroinflammatory and CNS Diseases Targeting Eicosanoids and Endocannabinoids Pathways to Attenuate Mitochondrial Dysfunction

Metabolic disruptions influencing vascular physiology, inflammation, and energy metabolism have been reported to increase the risk of Alzheimer's disease (AD) and other CNS disorders. Further, neuronal mitochondrial dysfunction is a critical process accelerating AD and other CNS disorders. Bioactive metabolites of fatty acids, including their oxygenated products (i.e., oxylipins) and their ethanolamide derivatives (i.e., acyl ethanolamides), are well-established players in cardiometabolic diseases and depression with both peripheral and central actions. These regulatory lipids modulate a variety of processes including inflammation, blood flow and neuronal cell proliferation. Lipid mediators derived from long chain omega-3 polyunsaturated fatty acids including eicosapentaenoic acid (EPA) and docosahexaenoic acid (DHA) have particularly potent anti-inflammatory and cardioprotective properties.

In the course of recent studies, alterations in both peripheral and central acylethanolamide and cytochrome p450/soluble epoxide hydrolase (CYP/sEH)-dependent oxylipins in AD and cognitive impairment have been discovered, with involvement of omega-3 fatty acid metabolism highlighted. It is contended that preserving levels of endogenous anti-inflammatory/pro-dilatory fatty acids by inhibiting their degrading enzymes, namely soluble epoxide hydrolase and fatty acid amide hydrolase (FAAH), while supplementing individuals with long-chain omega-3 fatty acid precursors will provide a novel and efficacious therapeutic strategy in the treatment of neuroinflammatory diseases.

Acylethanolamides include both cannabinoid receptor 1 (CB1), CB2 ligands, and related entourage compounds which are ligands for the capsaicin sensitive transient receptor potential cation channel subfamily V member 1 (TRPV1), and the peroxisome proliferator activated receptor alpha. These acylethanoloamides have anti-inflammatory and neuroprotective properties, being implicated in neuroinflammatory processes. In a recent study it was found AD patients to have lower levels of long-chain acylethanolamides, especially the EPA-derived EPEA in plasma, and oleic acid acylethanolamide (OEA) in both plasma and cerebrospinal fluid (CSF) compared to the healthy controls. Additionally, plasma EPEA showed a positive association with MoCA score in AD patients and a positive association with perceptual speed in cognitively normal elderly individuals, suggesting changes in acylethanolamide metabolism may precede AD pathology. Notably exogenous acylethanolamides are potent activators of the CB1 receptors with potential therapeutic benefit for neuroinflammatory disease treatment due to their neuroprotective and anti-inflammatory activity. The therapeutic preservation of acylethanolamide tone through FAAH inhibition has been shown to have therapeutic potential in the management of pain and disorders of the central nervous system.

Multiple cytochrome P450s (CYPs) transform unsaturated fatty acids into epoxide-containing metabolites with anti-inflammatory and vasodilatory properties. Degradation of these epoxy fatty acids by the soluble epoxide hydrolase (sEH) not only halt their anti-inflammatory action, but yield pro-inflammatory and vasoconstricting diols, a process primarily studied in cardiovascular disease. Intervention with sEH inhibitors increase epoxy fatty acids and decrease diols, with a net anti-inflammatory effect. In recent investigations in AD, higher plasma levels of the EPA sEH metabolite 17,18-DiHETE and higher CSF levels of linoleate-derived CYP metabolites 9(10)-EpOME and 12(13)-EpOME were found in AD patients. Omega-3 sEH metabolites are particularly potent regulators of blood vessel tone and vascular inflammation and sEH inhibitors have been suggested to improve outcomes for both cardiovascular and neurodegenerative diseases.

Linoleate-derived CYP metabolites elevated in the spinal tissue of burn victims have also been shown to activate the capsaicin sensitive transient vanilloid receptor type 1 (TRPV1). Activation of the TRPV1 can rescue neuronal function from Aβ-induce impairment and can alleviates cognitive and synaptic plasticity impairments in the APP23/PS45 mouse model of AD. Considering that acylethanolamides are also potent activators of the TRPV1, the observed increase in linoleate CYP metabolites may compensate for the AD-related decrease in these ethanolamides. This hypothesis is supported by the positive correlation of CSF linoleate epoxides with the MoCA score in AD patients, suggesting elevation of epoxy fatty acids in the central nervous system is potentially beneficial in AD. Those results implicate changes in both peripheral and central CYP/sEH metabolism in association with AD and cognitive impairment. They also are concordant with genotype variants in the CYP/sEH pathway altering AD risk. In particular, a single nucleotide polymorphisms (SNPs) in the CYP2J2 promoter region that reduces gene expression by ˜50% and appears to increase the ApoE4-independent AD risk. Several functional SNPs are also known to influence sEH activity and/or expression and influence disease risk. Relevant to AD-associated pathologies, loss of function sEH mutations protect neurons from ischemia-induced death and may alter the risk of vascular cognitive impairment. Additionally, postmortem brains from human subjects with AD have higher sEH levels, when compared to age-matched healthy controls.

Treatment with sEH inhibitors can reverse microglia and astrocyte reactivity and immune pathway dysregulation in mouse AD models. Additionally, brain sEH was positively associated with AD in a replicated protein wide-association study of AD. Therefore, reducing sEH function appears to be protective, and supports sEH as a valuable therapeutic target for the treatment and investigation of neuro-inflammatory pathologies including AD.

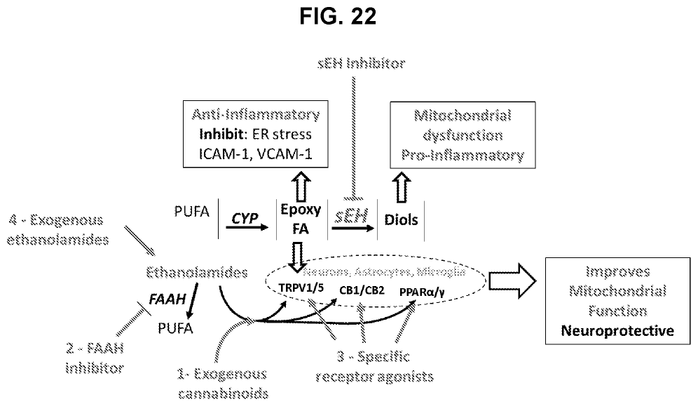

Based on the new findings and the relevant associated body of literature, it is concluded that a therapy that reduces sEH activity and preserves high levels of acylethanolamides, or the utilization of exogenous cannabinoids, will be useful (alone or in combination) in treating multifactorial neuroinflammatory diseases. Strategies for targeting cytochrome P450 (CYP)/soluble epoxide hydrolase (sEH) and ethanolamide pathways are presented in FIG. 22. FIG. 22 shows that polyunsaturated fatty acids are metabolized to epoxy fatty acids by CYP. Epoxy fatty acids are anti-inflammatory and can limit mitochondrial dysfunction and endoplasmic reticulum stress and can activate TRPV1. TRPV1 activation rescues neuronal function from Aβ-induced impairment. Epoxy fatty acids are converted to corresponding diols by sEH. A list of available sEH inhibitors is presented in Table 15. Inhibition of sEH has been shown to limit mitochondrial damage and preserve function following ischemic injury. Fatty acid ethanolamides directly activate TRPVS and CB1/CB2 receptors, and also PPARα and PPARγ, which have been shown to protect mitochondrial function. The acylethanolamides pathway can be targeted in four primary ways, indicated by the red arrows: (1) exogenous cannabinoids such as THC or CBD; (2) fatty acids amide hydrolase (FAAH) inhibitor; (3) specific receptor agonists; and (4) exogenous acylethanolamides. A list of available drugs utilizing those approaches is presented in Table 16. Moreover, the use of sEH/FAAH inhibition with either EPA, DHA, or EPA+DHA supplementation will provide additional therapeutic benefits by amplifying the availability of anti-inflammatory metabolite precursors, resulting in increased omega-3 fatty acid-derived epoxy fatty acids and acyl-ethanolamides in tissues.

TABLE 15
Drugs developed to inhibit sEH activity

| Active Ingredient | Indications | Status |
|---|---|---|
| EC5026 (EicOsis | Pain treatment | FDA approved IND |
| Human Health Inc) | | application to initiate Phase |
| | | 1b clinical trials |
| GSK2256294 | Insulin resistance in | Phase 2 |
| (GlaxoSmithKline) | peripheral tissues | |
| AR9281 (Arete | Hypertension, glucose | Phase 2 completed |
| Therapeutics) | tolerance | |
| TPPU | | |
| t-TUCB | | |

Italicized drugs are in clinical trials.

TABLE 16
Drugs developed to target ethanolamides (class of endocannabinoids)
pathway.

| Exogenous | FAAH | 3CB1/CB2, | |
|---|---|---|---|
| cannabinoids | inhibitors | TRPV1 agonists | Ethanolamides |
| THC— | SSR411298 | Nabilone | Um-PEA |
| Dronabinol | (Sanofi) | (Valeant | (FSD201) |
| (Axim ® | Pain in cancer; | Pharmaceuticals) | (FSD |
| Biotechnologies) | anxiety, | Nausea and | Pharma, Inc) |
| Psychotropic | cognitive | emesis associated | Chronic pain |
| nausea and emesis | function, sleep, | with cancer | |
| associated with | pain, and | chemotherapy | |
| cancer | somatic | | |
| chemotherapy | symptoms | | |
| | related to | | |
| | depression | | |
| CBD—Epidiolex | PF-04457845 | NEO6860 | OEA |
| (GW | (Pfizer) | (Neomed | (RiduZone) |
| Pharmaceuticals) | Cannabis use | Institute) | inflammation |
| Non-psychotropic | disorders; Pain | Pain, | and oxidative |
| Lennox-Gastaut | in osteoporosis | osteoarthritis, | stress in |
| syndrome and | | asthma | obesity |
| Dravet syndrome | | | |
| THC + CBD | JNJ-42165279 | | |
| Spasticity and pain | (Janssen | | |
| in multiple | Pharmaceutica) | | |
| sclerosis | URB597 (KDS- | | |
| | 4103) | | |
| | (Kadmus | | |
| | Pharmaceuticals, | | |
| | Inc.) | | |
| | ST4070 | | |
| | (Alfasigma) | | |

Bolded drugs are FDA approved;
Italicized drugs are in clinical trials.

What is claimed:

1. A method for the classification and treatment of a CNS disease in a subject, the method comprising one or more of the following:
(a) identifying and stratifying subjects afflicted with a CNS disease into subgroups based on their metabolic profiles, biomarker metabolites and ratios of biomarker metabolites that define unique metabolic conditions related to change in mitochondrial function and common identity among subgroups of subjects;
(b) evaluating the trajectory of disease within each stratified subgroup of subjects and their response to a therapeutic treatment;
(c) identifying defects in transport and/or biosynthesis breakdown of biomarker metabolites within a metabolic pathway or across metabolic pathways using ratios of biomarker metabolites to inform about changes in enzyme activities or transporters; and
(d) identifying genetic bases of metabolic profile characteristics or defects (SNPs/genetic variants in key enzymes and transporters) using mGWAS analysis.

2. The method of claim 1, further comprising one or more of the following:
(a) using combined metabotype and genotype data to better stratify subjects with neuropsychiatric diseases and to inform about mechanisms and treatment selection;
(b) suggesting a therapeutic approach to correct metabolic defects in metabolic profile in stratified subgroups of subjects; and
(c) comparing and contrasting metabolic defects noted in inborn errors of metabolism that have neurological and CNS deficits and using knowledge gained in treatment of inborn errors of metabolism to inform treatment for CNS diseases.

3. The method of claim 1 or 2, further comprising administering to the stratified subgroups of subjects an effective amount of a therapy to prevent and/or treat the CNS disease affected by specified genetic metabolic defects.

4. A method for stratifying and treating a subject having a neurological disorder, or at risk of developing a neurological disorder, based on the subject's mitochondrial metabolic profile, the method comprising:
obtaining a sample from the subject;
measuring the concentration levels and calculating the ratios of one or more mitochondrial biomarker metabolites in the sample, wherein the one or more mitochondrial biomarker metabolites are selected from carnitine, short-chain acylcarnitines, medium-chain acylcarnitines, or long-chain acylcarnitines; ketone bodies; amino acids, branched chain amino acids; biogenic amines; glycerophospholipids; sphingolipids; short-chain fatty acids; endocannabinoids; eicosanoids; other metabolites of glycolysis, TCA cycle, fatty acid beta-oxidation, urea cycle, or ketogenesis; or combinations thereof;
determining if the subject has a mitochondrial metabolic defect related to disrupted acylcarnitine homeostasis, TCA cycle, glycolysis, fatty acid beta-oxidation, ketogenesis, urea cycle, or combinations thereof based on the measured concentration levels and calculated ratios of the one or more mitochondrial biomarker metabolites in the sample as compared to a control sample; and
stratifying the subject into a subgroup of subjects, wherein an individual subgroup of subjects is defined by a unique and specific mitochondrial metabolic profile based on the measured concentration levels and calculated ratios of the one or more mitochondrial biomarker metabolites in the sample as compared to a control sample and the mitochondrial metabolic defect determined for the subject.

5. The method of claim 4, further comprising administering to the subgroup of subjects an effective amount of a therapy to treat the neurological disease, wherein the therapy is determined by the unique and specific mitochondrial metabolic profile of the subgroup of subjects.

6. The method of claim 4, wherein the biomarker metabolite comprises one or more of:
Carnitine; Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); CS-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine);
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine);
Long-Chain Acylcarnitines: C14 (tetradecanoylcarnitine); C14:1 (tetradecenoylcarnitine); C14:1-OH (hydroxytetradecenoylcarnitine); C14:2 (tetradecadienylcarnitine); C14:2-OH (hydroxytetradecadienylcarnitine); C16 (hexadecanoylcarnitine); C16-OH (hydroxyhexadecanoylcarnitine); C16:1 (hexadecenoylcarnitine); C16:1-OH (hydroxyhexadecenoylcarnitine); C16:2 (hexadecadienylcarnitine); C16:2-OH (hydroxyhexadecadienylcarnitine); C18 (octadecanoylcarnitine); C18:1 (octadecenoylcarnitine; C18:1-OH (hydroxyoctadecenoylcarnitine); or C18:2 (octadecadienylcarnitine);
or combinations thereof.

7. The method of claim 6, wherein the mitochondrial metabolic defect is related to disrupted acylcarnitine homeostasis and comprises:
lower concentration levels of all acylcarnitines and higher ratios of carnitine/C3:0, carnitine/C5:0, carnitine/C10:0, carnitine/C16:0, and carnitine/C18:1 acylcarnitines;
lower ratios of short and medium chain vs. long chain acylcarnitines (including C3:0/C16:0, C5:0/C16:0, C10:0/C16:0, C3:0/C18:1, C5:0/C18:1, and C10:0/C18:1);
lower ratios of short chain vs. medium and long chain acylcarnitines (including C3:0/C10:0, C5:0/C10:0, C3:0/C16:0, C5:0/C16:0, C3:0/C18:1 and C5:0/C18:1); or
lower ratios of odd-numbered short chain acylcarnitines vs. even-numbered short chain acylcarnitines (e.g., C6) and medium and long chain acylcarnitines (including C3:0/C6:0, C5:0/C6:0, C3:0/C10:0, C5:0/C10:0, C3:0/C16:0, C5:0/C16:0, C3:0/C18:1 and C5:0/C18:1).

8. The method of claim 6, wherein the mitochondrial metabolic defect is related to disrupted acylcarnitine homeostasis and comprises:
Short-Chain Acylcarnitines: C0 (carnitine); C2 (acetylcarnitine); C3 (propionylcarnitine); C3-OH (hydroxypropionylcarnitine); C3:1 (propenoylcarnitine); C3-DC (C4-OH) (hydroxybutyrylcarnitine); C4 (butyrylcarnitine); C4:1 (butenylcarnitine); C5 (valerylcarnitine); C5-M-DC (methylglutarylcarnitine); C5:1 (tiglylcarnitine); C5:1-DC (glutaconylcarnitine); C5-OH (C3-DC-M) (hydroxyvalerylcarnitine or methylmalonylcarnitine); or C5-DC (C6-OH) (glutarylcarnitine or hydroxyhexanoylcarnitine); and
Medium-Chain Acylcarnitines: C6 (C4:1-DC) (hexanoylcarnitine or fumarylcarnitine); C6:1 (hexenoylcarnitine); C7-DC (pimelylcarnitine); C8 (octanoylcarnitine); C9 (nonaylcarnitine); C10 (decanoylcarnitine); C10:1 (decenoylcarnitine); C10:2 (decadienylcarnitine); C12 (dodecanoylcarnitine); C12-DC (dodecanedioylcarnitine); or C12:1 (dodecenoylcarnitine).

9. The method of claim 5, wherein the therapy comprises one or more compounds selected from peroxisome proliferator-activated receptor (PPAR) agonists, PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acid: Even (C₆-C₁₂), medium chain fatty acid: Odd chain fatty acids (C₇, C₉), branched chain amino acids, carnitine, acetyl carnitine, propionylcarnitine, short chain acylcarnitines (C₂₋₅), cofactors NAD Flavin FAD, and combinations thereof.

10. The method of claim 4, wherein the mitochondrial metabolic defect comprises a deficiency in amino acids (e.g., branched chain amino acids) and/or short chain fatty acids.

11. The method of claim 9, wherein the therapy comprises one or more compounds selected from branched chain amino acids, propionic acid, ketone bodies, short chain acylcarnitines (C₂₋₅), medium chain acylcarnitines, analogs thereof, and combinations thereof.

12. The method of claim 4, wherein the neurological disorder is a CNS disorder, depression, or treatment resistant depression.

13. A method for stratifying and treating a subject having a neurological or CNS disorder, or at risk of developing a CNS or neurological disorder, based on the subject's mitochondrial metabolic profile, the method comprising:
obtaining a sample from the subject;
measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof in the sample; and
determining if the subject has a mitochondrial metabolic defect related to disrupted acylcarnitine homeostasis, TCA cycle, glycolysis, fatty acid beta-oxidation, ketogenesis, urea cycle, or combinations thereof based on the measured expression level and/or activity of mitochondrial enzymes and/or transporters in the sample as compared to a control sample.

14. The method of claim 13, further comprising stratifying the subject into a subgroup of subjects, wherein an individual subgroup of subjects is defined by a unique and specific mitochondrial metabolic profile based on the measured expression level and activity of mitochondrial enzymes and/or transporters in the sample as compared to a control sample and the mitochondrial metabolic defect determined for the subject.

15. The method of claim 14, further comprising administering to the subgroup of subjects an effective amount of a therapy to treat the neurological disease, wherein the therapy is determined by the unique and specific mitochondrial metabolic profile of the subgroup of subjects.

16. The method of claim 13, wherein the mitochondrial enzyme and/or transporter comprises gamma-butyrobetaine hydroxylase 1 (BBOX1), organic cation transporter novel family member 2 (OCTN2), very long chain acylCoA dehydrogenase (VLCAD), medium chain acylCoA dehydrogenase (MCAD), short chain acylCoA dehydrogenase (SCAD), carnitine palmitoyltransferase1/2 (CPT1/2), carnitine-acylcarnitine translocase (CACT), carnitine octanoyltransferase, acetyl-CoA carboxylase1/2 (ACC1/2), ATP citrate synthase (ACLY), peroxisome proliferator-activated receptor (PPARα/PPARγ), or combinations thereof.

17. The method of claim 16, wherein the mitochondrial metabolic defect is related to disrupted acylcarnitine homeostasis and comprises:
CPT defects: lower concentration levels of all acylcarnitines and higher ratios of carnitine/C3:0, carnitine/C5:0, carnitine/C10:0, carnitine/C16:0, and carnitine/C18:1 acylcarnitines;
VLCAD defects: lower ratios of short and medium chain vs. long chain acylcarnitines (including C3:0/C16:0, C5:0/C16:0, C10:0/C16:0, C3:0/C18:1, C5:0/C18:1, and C10:0/C18:1);
MCAD defects: lower ratios of short chain vs. medium and long chain acylcarnitines (including C3:0/C10:0, C5:0/C10:0, C3:0/C16:0, C5:0/C16:0, C3:0/C18:1 and C5:0/C18:1); or
SCAD defects: lower ratios of odd-numbered short chain acylcarnitines vs. even-numbered short chain acylcarnitines (e.g., C6) and medium and long chain acylcarnitines (including C3:0/C6:0, C5:0/C6:0, C3:0/C10:0, C5:0/C10:0, C3:0/C16:0, C5:0/C16:0, C3:0/C18:1 and C5:0/C18:1).

18. The method of claim 15, wherein the therapy comprises one or more compounds selected from peroxisome proliferator-activated receptor (PPAR) agonists, PPARα agonists, PPARγ agonists, PPARδ agonists, PPAR dual agonists, PPAR pan agonists, metformin, triheptanoin, ketone bodies, short chain fatty acids, medium chain fatty acids, medium chain fatty acid: Even (C₆-C₁₂), medium chain fatty acid: Odd chain fatty acids (C₇, C₉), branched chain amino acids, carnitine, acetyl carnitine, propionylcarnitine, short chain acylcarnitines (C₂₋₅), cofactors NAD Flavin FAD, and combinations thereof.

19. The method of claim 16, wherein the mitochondrial metabolic defect is related to disrupted acylcarnitine homeostasis and comprises disrupted BBOX1, OCTN2, and/or CPT1/2 expression and/or activity.

20. A method for treating a CNS or neuropsychiatric disease in a subject, the method comprising:
obtaining a sample from the subject;
determining the presence, concentration levels, and ratios of one or more biomarker metabolites related to mitochondrial function in the sample from the subject;
comparing the presence, concentration levels, and ratios of one or more biomarker metabolites related to mitochondrial function in the sample from the subject to the presence, concentration levels, and ratios of the one or more biomarker metabolites in a control sample; and
determining if the subject has a CNS or neuropsychiatric disorder, or has an increased risk of developing a CNS or neuropsychiatric disorder when the concentration levels and ratios of the one or more biomarker metabolites in the sample from the subject are different from (greater than or less than) the concentration levels and ratios of the one or more biomarker metabolites in a control sample.

21. The method of claim 20, further comprising:
stratifying the subject into a subgroup of subjects based on the concentration levels and ratios of the one or more biomarker metabolites related to mitochondrial function in the sample, wherein each subgroup of subjects is defined by a unique and specific mitochondrial metabolic profile; and
administering to the subgroup of subjects an effective amount of a therapy to treat the CNS or neuropsychiatric disease, wherein the therapy is determined by the unique and specific mitochondrial metabolic profile of each subgroup of subjects.

22. A method for targeting mitochondrial pathways related to oxidative stress, mitochondrial biogenesis, and mitochondrial membrane permeability and dynamics in a subject suffering from, or at risk of suffering from, one or more neurodegenerative diseases, the method comprising:
obtaining a sample from the subject and determining the concentration levels and ratios of one or more mitochondrial biomarker metabolites in the sample from the subject;
determining if the subject has a neurodegenerative disease, or has an increased risk of developing a neurodegenerative disease when the concentration levels and ratios of the one or more mitochondrial biomarker metabolites in the sample from the subject are different from (greater than or less than) the concentration levels and ratios of the one or more mitochondrial biomarker metabolites in a control sample;
stratifying the subject into a subgroup of subjects based on the concentration levels and ratios of the one or more mitochondrial biomarker metabolites in the sample, wherein each subgroup of subjects is defined by a unique and specific mitochondrial metabolic profile; and
administering to the subgroup of subjects an effective amount of a therapy to treat the neurodegenerative disease, wherein the therapy is determined by the unique and specific mitochondrial metabolic profile of the subgroup of subjects.

23. A method for preparing and analyzing a sample containing a biomarker metabolite useful for the analysis and identification of metabolic changes associated with a CNS or neuropsychiatric disease in a subject, the method comprising:
obtaining a sample from a subject;
performing metabolic analysis on the sample to detect the presence and concentration of one or more biomarker metabolites;
comparing the presence and concentration levels of one or more biomarker metabolites in the sample from the subject to the concentration levels of the one or more biomarker metabolites in a control sample;
determining whether the presence and concentration levels of one or more biomarker metabolites in the sample from the subject correlate with the incidence of a CNS or neuropsychiatric disease, or an increased risk of a CNS or neuropsychiatric disease.

24. A method for stratifying and treating a subject having a neurological disorder, or at risk of developing a neurological disorder, based on the subject's mitochondrial metabolic profile, the method comprising:
obtaining a sample from the subject;
measuring the concentration levels and calculating the ratios of one or more mitochondrial biomarker metabolites in the sample, wherein the one or more mitochondrial biomarker metabolites are selected from carnitine, short-chain acylcarnitines, medium-chain acylcarnitines, or long-chain acylcarnitines; ketone bodies; amino acids, branched chain amino acids; biogenic amines; glycerophospholipids;
sphingolipids; short-chain fatty acids; endocannabinoids; eicosanoids; other metabolites of glycolysis, TCA cycle, fatty acid beta-oxidation, urea cycle, or ketogenesis; or combinations thereof;
measuring the expression level and/or activity of one or more mitochondrial enzymes and/or transporters involved in acylcarnitine biosynthesis and transport, TCA cycle, glycolysis, fatty acid beta-oxidation, ketogenesis, urea cycle, or combinations thereof in the sample; and
determining if the subject has a mitochondrial metabolic related to disrupted acylcarnitine homeostasis, TCA cycle, glycolysis, fatty acid beta-oxidation, ketogenesis, urea cycle, or combinations thereof based on the measured concentration levels and calculated ratios of the one or more mitochondrial biomarker metabolites in the sample as compared to a control sample and/or genetic defect in one or more mitochondrial enzymes and/or transporters involved in acylcarnitine biosynthesis and transport, TCA cycle, glycolysis, fatty acid beta-oxidation, ketogenesis, urea cycle, or combinations thereof; and
stratifying the subject into a subgroup of subjects, wherein an individual subgroup of subjects is defined by a unique and specific mitochondrial metabolic profile based on the measured concentration levels and calculated ratios of the one or more mitochondrial biomarker metabolites in the sample as compared to a control sample and the mitochondrial metabolic defect determined for the subject;
administering to the subgroup of subjects an effective amount of a therapy to treat the neurological disease, wherein the therapy is determined by the unique and specific mitochondrial metabolic profile of the subgroup of subjects.

25. The method of claim 32, wherein the therapy comprises one or more compounds selected from branched chain amino acids, propionic acid, ketone bodies, short chain acylcarnitines (C₂₋₅), medium chain acylcarnitines, analogs thereof, and combinations thereof.

26. The method of claim 32, wherein the neurological disorder is a CNS disorder, depression, or treatment resistant depression.

27. The method of any one of claims 1-26, wherein the therapy comprises one or more repurposed compounds that improve mitochondrial energetics to treat CNS or neuropsychiatric diseases.

28. The method of any one of claims 1-27, wherein the therapy comprises one or more repurposed compounds that modulate mitochondrial energetics to treat neurodegenerative diseases.

29. The method of any one of claims 1-28, wherein the therapy comprises one or more of HU-210; CP 55940; Win 55212-2; anandamide; 2-AG; Noladin ether; virodhamine; oleoylethanolamide; palmitoylethanolamide; PPARγ Agonists; PPARβ/δ Agonists; dual and pan PPAR agonists; chiglitazar (CS038); AVE0847; aleglitazar (R1439); 5-substituted 2-benzoylamino-benzoic acid derivatives (BVT-142); O-arylmandelic acid derivatives; azaindole-α-alkyloxyphenylpropionic acid; amide substituted/α-substituted β-phenylpropionic acid derivatives; 2-alkoxydihydrocinnamate derivative; α-aryloxy-α-methylhydrocinnamic acids (LYS1029); TZD18; α-aryloxyphenyl acetic acid derivatives; PLX249; muraglitazar; mesaglitazar; naveglitazar; ragaglitazar; farglitazar; imiglitazar; netoglitazone; compound 3q JTT-501; MK0767; KRP-297; AZD6610; (atorvastatin+ezetimibe+fenofibrate); (fenofibrate+pravastatin sodium); (fenofibrate+rosuvastatin calcium); (fenofibrate+rosuvastatin); (fenofibrate+simvastatin); (fenofibrate+pitavastatin); (gliclazide+metform in hydrochloride+pioglitazone hydrochloride); (gliclazide+metformin hydrochloride+rosiglitazone); (gliclazide SR+metformin hydrochloride SR+pioglitazone hydrochloride); (gliclazide SR+metformin SR+pioglitazone); glimepiride+metformin SR+pioglitazone; (metformin ER+pioglitazone); (metformin hydrochloride+pioglitazone); (metformin hydrochloride+rosiglitazone maleate); (metformin hydrochloride+pioglitazone hydrochloride); (fenofibrate+metformin hydrochloride); (gliclazide+rosiglitazone); (glimepiride+pioglitazone); (alogliptin benzoate+pioglitazone hydrochloride); ciprofibrate; fenofibrate; gemfibrozil; bezafibrate SR; clinofibrate; clofibrate; clofibrate; choline fenofibrate; saroglitazar; lobeglitazone; zaltoprofen; pemafibrate; pemafibrate+tofogliflozin; MA-0211; REN-001; EHP-101; ZYH-7; elafibranor; NC-2400; MA-0217; T-3D959; CHS-131; efatutazone; OMS-405; seladelpar lysine; leriglitazone hydrochloride; CS-038; AU-9; BIO-201; BIO-203; BR-101549; CDIM-9; CNB-001; ELB-00824; ETI-059; KR-62980; MA-0204; PLX-300; RB-394; SR-10171; sulindac; ZG-0588; A-91; AIC-47; CDE-001; CDIM-1; CDIM-5; CDIM-7; OP-601; (azilsartan+pioglitazone hydrochloride); ADC-3277; ADC-8316; ARH-049020; arhalofenate; ATX-08001; AVE-0897; AZD-6610; BP-1107; CG-301269; CLC-3000; CLC-3001; CNX-013B2; CP-778875; CS-1050; CXR-1002; DB-900; DJ-5; DRF-10945; etalocib; farglitazar; GED-0507; indeglitazar; K-111; KD-3010; KD-3020; KRP-101; LY-518674; LY-518674; mesalamine; NIP-222; NP-774; NS-220; PAM-1616; PBI-4547; PBI-4547; PBI-4547; peroxibrate; PN-2034; PPM-201; PPM-202; romazarit; metformin; triheptanoin; ketone bodies; short chain fatty acids; methanoic acid; ethanoic acid; propanoic acid; butanoic acid; 2-methyl propanoic acid; pentanoic acid; 3-methyl butanoic acid; medium chain fatty acids; medium even (C6-C12) chain fatty acids; medium odd (C7, C9) chain fatty acids; fatty acid ethanolamides; cannabinoids; branched chain amino acids; nicotinamide adenine dinucleotide (NAD+/NADH, NADP+/NADPH); riboflavin; flavin adenine dinucleotide (FAD); EC5026; GSK2256294; AR9281; TPPU; t-TUCB; Dronabinol; Epidiolex; Δ9-tetrahydrocannabinol; cannabidiol; Δ9-tetrahydrocannabinol+cannabidiol; SSR411298; PF-04457845; JNJ-42165279; URB597 (KDS-4103); ST4070 (Alfasigma); Nabilone; Um-PEA (FSD201); NEO6860; OEA (RiduZone); or combinations thereof.

30. The method of any one of claims 1-29, wherein therapy comprises one or more of: Δ⁹-tetrahydrocannabinol (Δ⁹-THC; Dronabinol); Δ⁸-tetrahydrocannabinol (Δ⁸-THC); exo-tetrahydrocannabinol (Exo-THC); Δ⁹-tetrahydrocannabinol naphtoylester (Δ⁹-THC-NE); Δ⁸-tetrahydrocannabinol naphtoylester (Δ⁸-THC-NE); exo-tetrahydrocannabinol naphtoylester (Exo-THC-NE); Δ⁹-tetrahydrocannabinolic acid (THCA-A, THCA-B); Δ⁸-tetrahydrocannabinolic acid (Δ⁸-THCA-A, Δ⁸-THCA-B); (−)-cannabidiol ((−)-CBD)/(+)-cannabidiol ((+)-CBD); cannabidiol-2′,6′-dimethyl ether (CBDD); 4-monobromo cannabidiol (4-MBO-CBD); cannabidiolic acid (CBDA); cannabiquinone (CBQ); nabilone; cannabivarin (CBNV); cannabivarinic acid (CBNVA); cannabivarin naphtoylester (CBNV-NE); Δ⁹-tetrahydrocannabivarin (Δ⁹-THCBV); Δ⁸-tetrahydrocannabivarin (Δ⁸-THCBV); Δ⁹-tetrahydrocannabivarin naphtoylester (Δ⁹-THCV-NE); Δ⁸-tetrahydrocannabivarin naphtoylester (Δ⁸-THCV-NE); Δ⁹-tetrahydrocannabivarinic acid (Δ⁹-THCVA); Δ⁸-tetrahydrocannabivarinic acid (Δ⁸-THCVA); (−)-cannabidivarin ((−)-CBDV)/(+)-cannabidivarin ((+)-CBDV)); cannabidivarinic acid (CBDVA); cannabidivarin quinone (CBQV); cannabidibutol (CBDB); cannabidibutolic acid (CBDBA); cannabidibutol naphtoylester (CBDB-N E); Δ⁹-tetrahydrocannabidutol (Δ⁹-THCBDB); Δ⁸-tetrahydrocannabidutol (Δ⁸-THCBDB); Δ⁹-tetrahydrocannabidutolic acid (Δ⁹-THCBDBA); Δ⁸-tetrahydrocannabidutolic acid (Δ⁸-THCBDBA); Δ⁹-tetrahydrocannabidutol naphtoylester (Δ⁹-THCB-NE); Δ⁸-tetrahydrocannabidutol naphtoylester (Δ⁸-THCB-NE); cannabibutol (CBB); cannabibutolic acid (CBBA); Δ⁹-tetrahydrocannabibutol (Δ⁹-THCB); Δ⁸-tetrahydrocannabibutol (Δ⁸-THCB); Δ⁹-tetrahydrocannabibutoic acid (Δ⁹-THCBA); Δ⁸-tetrahydrocannabibutolic acid (Δ⁸-THCBA); Δ⁹-tetrahydrocannabibutol naphtoylester (Δ⁹-THCB-NE); Δ⁸-tetrahydrocannabibutol naphtoylester (Δ⁸-THCB-NE); cannabinol (CBN); cannabinolic acid (CBNA); 3-butylcannabinol (CBNB); 3-butylcannabinolic acid (CBNBA); cannabielsoin (CBE); cannabicitran (CBT); cannabicyclol (CBL); cannabicyclolic acid (CBLA); cannabicyclol butyl (CBLB); cannabicyclol butyric acid (CBLBA); cannabicyclolvarin (CBLV); cannabicyclolvarinic acid (CBLVA); cannabigerol (CBG); cannabigerolic acid (CBGA); cannabigerol butyl (CBGB); cannabigerol butyric acid (CBGBA); cannabichromene (CBC); cannabichromenic acid (CBCA); cannabichromene butyl (CBCB); cannabichromene butyric acid (CBCBA); cannabigerivarin (CBGV); cannabigerivarinic acid (CBGVA); cannabichromevarin (CBCV); cannabichromevarinic acid (CBCVA); other cannabinoids, or pharmaceutically acceptable salts, acids, esters, amides, hydrates, solvates, prodrugs, isomers, stereoisomers, tautomers, derivatives thereof, or combinations thereof.

31. The method of any one of claims 1-30, wherein the therapy further comprises an anti-depressant selected from selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof.

32. The method of any one of claims 1-31, wherein the therapy further comprises one or more of citalopram, escitalopram, duloxetine, fluoxetine, paroxetine, sertraline, trazodone, lorazepam, oxazepam, aripiprazole, clozapine, haloperidol, olanzapine, quetiapine, risperidone, ziprasidone, amitriptyline, amoxapine, desipramine, doxepin, imipramine, nortriptyline, protriptyline, trimipramine, or combinations thereof.

33. The method of any one of claims 1-32, wherein the therapy further comprises ketamine or esketamine.

34. The method of any one of claims 1-33, wherein the therapy comprises one or more compounds selected from cannabinoids, cannabinoid-like compounds, carnitine, L-acetyl carnitines, and combinations thereof.
